SEQUENCE LISTING (639 sequences)

SEQ ID NO: 1 - DNA, 60 bp, Artificial Sequence
  Synthetic
ttgagtcatttttatcacaataatcctactgtgaagctgtcgttgagaacttaggttggc

SEQ ID NO: 2 - DNA, 60 bp, Artificial Sequence
  Synthetic
ttcttcttcttatcttgttattacggttttattaattttgtagagggacagggagtgggc

SEQ ID NO: 3 - DNA, 60 bp, Artificial Sequence
  Synthetic
tagacctgaaatctttgtttttcctattgacaagggcttggtccgtctgttggccaggaa

SEQ ID NO: 4 - DNA, 60 bp, Artificial Sequence
  Synthetic
tttcacgagtcttcaagctttcaggctatcttctagtcaagatgagtgataagccagact

SEQ ID NO: 5 - DNA, 60 bp, Artificial Sequence
  Synthetic
tttaaatgtaacagatctgaaaacttaccagattgggtgggatacattctgtgtcaaatg

SEQ ID NO: 6 - DNA, 60 bp, Artificial Sequence
  Synthetic
tggattgatattacagatgtaaaacctggaaactatatcctaaaggtcagtgtaaacccc

SEQ ID NO: 7 - DNA, 60 bp, Artificial Sequence
  Synthetic
tatagacattctcacataagcccagttcatcaccatttcctcctttacctttcagtgcag

SEQ ID NO: 8 - DNA, 60 bp, Artificial Sequence
  Synthetic
tttatctcaagtgaacctggagaagcaacaataatggaccttctcccctagtcaaatagc

SEQ ID NO: 9 - DNA, 60 bp, Artificial Sequence
  Synthetic
cagcattcttacgtgagaatttcacttacgggagagttcttacaggatttcaagaaggaa

SEQ ID NO: 10 - DNA, 60 bp, Artificial Sequence
  Synthetic
ttgccttaacattgcaattgcatttaacagtgttacttttaacattgcctcgtggcctca

SEQ ID NO: 11 - DNA, 60 bp, Artificial Sequence
  Synthetic
tttctctctgcattgtgtagtgagtggtgtcacccaggactttcatgtgagaaaaagcac

SEQ ID NO: 12 - DNA, 60 bp, Artificial Sequence
  Synthetic
tcttcattcgccaggctttgaggccatttccctattcattaaagactaatgtttaaaaag

SEQ ID NO: 13 - DNA, 60 bp, Artificial Sequence
  Synthetic
ttatccctcaggatggtgattatagagaatttcccattcctgaacagttcaagacagcat

SEQ ID NO: 14 - DNA, 60 bp, Artificial Sequence
  Synthetic
tgaaaaggcggtactagttcagacactttggaagtttgtgttctgtttgttaaaactggc

SEQ ID NO: 15 - DNA, 60 bp, Artificial Sequence
  Synthetic
tagttaccattttcagtgttatttcaaaggttctttgaagaattttggggcagggcatca

SEQ ID NO: 16 - DNA, 60 bp, Artificial Sequence
  Synthetic
tttgttcgtggtagcaccttcaaagaaatccccgtgactgtctatagagaagtatgacaa

SEQ ID NO: 17 - DNA, 60 bp, Artificial Sequence
  Synthetic
ttgtgatttcatgtttgtaatctacaacttttcaaaagcattcagtcatggtctgctagg

SEQ ID NO: 18 - DNA, 60 bp, Artificial Sequence
  Synthetic
cacaagttttagcctttttcacaagggaactcatactgtctacacatcagaccatagttg

SEQ ID NO: 19 - DNA, 60 bp, Artificial Sequence
  Synthetic
ttcaacctgattgacacttttcccttgagaaaagaagagatggtcctacaagccgaaatg

SEQ ID NO: 20 - DNA, 60 bp, Artificial Sequence
  Synthetic
tccagatataaaatatggtgcaatatccacttcatcacttggagaaaaagtgctgagtgc

SEQ ID NO: 21 - DNA, 60 bp, Artificial Sequence
  Synthetic
tttcaatattttaactttttgtttttatttcttttagaaaaggccaatatacctatcgcg

SEQ ID NO: 22 - DNA, 60 bp, Artificial Sequence
  Synthetic
tcactataccatgctataggagactgggcaaaacctgtacaatgacaaccctggaagttg

SEQ ID NO: 23 - DNA, 60 bp, Artificial Sequence
  Synthetic
ttgtagatggcagttatgaatgtatatttatattttgattaagatttctattaacttttt

SEQ ID NO: 24 - DNA, 60 bp, Artificial Sequence
  Synthetic
ttcctgggattgatggagttaaaggtgacaaaggaaatccaggctggccaggagcacccg

SEQ ID NO: 25 - DNA, 60 bp, Artificial Sequence
  Synthetic
tctgtgtattttcaaatgttactatatattaaagcagaaatataaccaaaggttaaaaaa

SEQ ID NO: 26 - DNA, 60 bp, Artificial Sequence
  Synthetic
ttgggactgtgttagcaactattattgtggaataacccagaacattggatttataccaac

SEQ ID NO: 27 - DNA, 60 bp, Artificial Sequence
  Synthetic
tgagaatgcacgcgtgcatatgctacacatatgtgcttctcagttgcagaaaatgaactg

SEQ ID NO: 28 - DNA, 60 bp, Artificial Sequence
  Synthetic
tttggactaatacaattcaggaaagaaaaaacccaaaaaccaacctcattcacatatggc

SEQ ID NO: 29 - DNA, 60 bp, Artificial Sequence
  Synthetic
ttggcactgaggggaccagctggcccgatgggtctcacagggagacctggccctgtgcaa

SEQ ID NO: 30 - DNA, 60 bp, Artificial Sequence
  Synthetic
gcttgaaaaagattagcatacatctaatgtgaaaagaccacatttgattcaactgagacc

SEQ ID NO: 31 - DNA, 60 bp, Artificial Sequence
  Synthetic
tttagccttttagaatgattcatgtcaacacttaagacaaagttcatgaaaagtcccagc

SEQ ID NO: 32 - DNA, 60 bp, Artificial Sequence
  Synthetic
gtcgcgatgcctattttagtcaaagcggctgcgggcttggggacccggcccgggcagcgg

SEQ ID NO: 33 - DNA, 60 bp, Artificial Sequence
  Synthetic
tttgctgtagccttcagtgtcctgctgttaagctgtaaggatcacgtggggtacattttt

SEQ ID NO: 34 - DNA, 60 bp, Artificial Sequence
  Synthetic
ttgccatttggacttggtactcggcttagtgattagaggccctgaacaggtggtggtatc

SEQ ID NO: 35 - DNA, 60 bp, Artificial Sequence
  Synthetic
tgtggcagtaatggcaagacctacctcaaccactgtgaactgcatcgagatgcctgcctc

SEQ ID NO: 36 - DNA, 60 bp, Artificial Sequence
  Synthetic
ggctagagatatatcttaatgcaatccattttctgatggattgttacgagttggctatat

SEQ ID NO: 37 - DNA, 60 bp, Artificial Sequence
  Synthetic
tttgaaccggggacagagtctaggtgagctggggcttgggagctattagcgtagaggatc

SEQ ID NO: 38 - DNA, 60 bp, Artificial Sequence
  Synthetic
aagaaaaagagagggcatgggttgcggagccgacatcacggccggggtctttgctgttta

SEQ ID NO: 39 - DNA, 60 bp, Artificial Sequence
  Synthetic
ttctattttcgtataacattgtcaagtggaaacatgctgaaatctattaaaccatctttg

SEQ ID NO: 40 - DNA, 60 bp, Artificial Sequence
  Synthetic
tttttagatacagagacttggggaaattgcttttcctcttgaaccacagttctacccctg

SEQ ID NO: 41 - DNA, 60 bp, Artificial Sequence
  Synthetic
ttccaaatggaaactgctaatttttgaagcagaaggttgacagcttcagtaagatctcaa

SEQ ID NO: 42 - DNA, 60 bp, Artificial Sequence
  Synthetic
ttctactacactgcgggctctagctccccgactcatgcgaagagtgcccacgtataagag

SEQ ID NO: 43 - DNA, 60 bp, Artificial Sequence
  Synthetic
taaaagagccaagttcaaagaaccctagcacaaatttgctttgggattttcttttctgga

SEQ ID NO: 44 - DNA, 60 bp, Artificial Sequence
  Synthetic
tggtttcatcttaatattagttatatttgtaaccggtctgcttttgcgtaagccaaacac

SEQ ID NO: 45 - DNA, 60 bp, Artificial Sequence
  Synthetic
attaaaaacccttttcctatgtttattgtatacaagaattatgcaataaaatttctttat

SEQ ID NO: 46 - DNA, 60 bp, Artificial Sequence
  Synthetic
gctttcctgtttctaaccttaggaaaccagaatagcgtttggcagacacgacgttttcag

SEQ ID NO: 47 - DNA, 60 bp, Artificial Sequence
  Synthetic
tgaagaatgcaaattgcacatcagattttgaggaatactttgccaaaagaaaactggagg

SEQ ID NO: 48 - DNA, 60 bp, Artificial Sequence
  Synthetic
ttttcagctataacacggattcccgccagacgtgtgctaacaacagacaccagtgctcgg

SEQ ID NO: 49 - DNA, 60 bp, Artificial Sequence
  Synthetic
gctgaaaatgaactttatgaaccttttccaagttgatctatccagtgacgtggcctggtg

SEQ ID NO: 50 - DNA, 60 bp, Artificial Sequence
  Synthetic
gctgttttgagtgttgatgaaaagcaatgcaattatgccaaacagtattgagcagaataa

SEQ ID NO: 51 - DNA, 60 bp, Artificial Sequence
  Synthetic
gtgttttggaaatatttgctgtgtctcaggggattgtaggaatacgaggagttttcagca

SEQ ID NO: 52 - DNA, 60 bp, Artificial Sequence
  Synthetic
ggttctttacaaatggtattttgatagatactggattgtgtttgtgccatatttgtgcca

SEQ ID NO: 53 - DNA, 60 bp, Artificial Sequence
  Synthetic
agatggctgactactaacaggtcattgccaggtgtatttctatactctttgaagaataac

SEQ ID NO: 54 - DNA, 60 bp, Artificial Sequence
  Synthetic
tcttgccttaacatcccttgcatttggctgcaaagaaatctgcttggaagaaggggttac

SEQ ID NO: 55 - DNA, 60 bp, Artificial Sequence
  Synthetic
tctcctatatatgaatgatgactttgaaggaggagaattcatattcacagagatggatgc

SEQ ID NO: 56 - DNA, 60 bp, Artificial Sequence
  Synthetic
ttgtggtttgaatgttcataagcagtgttccaagatggtcccaaatgactgtaagccaga

SEQ ID NO: 57 - DNA, 60 bp, Artificial Sequence
  Synthetic
ttcttcttgccaagcaatccaaagatgaactttctgaagcccgagaacttggcaacatgg

SEQ ID NO: 58 - DNA, 60 bp, Artificial Sequence
  Synthetic
ttcatagatgttgacaatttcctgactaatccacagaccctcaatctactgattgcagaa

SEQ ID NO: 59 - DNA, 60 bp, Artificial Sequence
  Synthetic
ctccgttattttagtgtgcttccttctcttgtctggatttctgcattgccctaggaagtc

SEQ ID NO: 60 - DNA, 60 bp, Artificial Sequence
  Synthetic
ggggagttgatagtctcataaaactaatttggcttcaagtttcatgaatctgtaactaga

SEQ ID NO: 61 - DNA, 60 bp, Artificial Sequence
  Synthetic
tgccgcctgtccgtatcagcaggatgctactgctgctgctgatacctggggaattgccag

SEQ ID NO: 62 - DNA, 60 bp, Artificial Sequence
  Synthetic
tttggagaaggggatggcattattagagagttatgcatctcatatggaggcgttggtcca

SEQ ID NO: 63 - DNA, 60 bp, Artificial Sequence
  Synthetic
gacagtttatttgttgagagtgtgaccaaaagttacatgtttgcacctttctagttgaaa

SEQ ID NO: 64 - DNA, 60 bp, Artificial Sequence
  Synthetic
ctgctcaacatccggagggaattcattgagaaatatgacaagtctctccaccaagccatt

SEQ ID NO: 65 - DNA, 60 bp, Artificial Sequence
  Synthetic
ttgtctttggtgaagggtctgctgtgcaccatcccccatcctacgtggcccacctggcct

SEQ ID NO: 66 - DNA, 60 bp, Artificial Sequence
  Synthetic
tttatgggactgtgtaaagtagctaagtcggttgaaatgctgatcctgggtcgcttggtt

SEQ ID NO: 67 - DNA, 60 bp, Artificial Sequence
  Synthetic
tcagcggcagcagccgccgccgagatgtcccggcgaaagcaaagcaaaccccggcagatc

SEQ ID NO: 68 - DNA, 60 bp, Artificial Sequence
  Synthetic
tttgccaagcattgcgtgaagtgcaacaaggacaagtgtgaaccatgagaagtatgacaa

SEQ ID NO: 69 - DNA, 60 bp, Artificial Sequence
  Synthetic
cttcggggagctcatcatgtctggcaagaacatgcggctgagctctctcgcgctctccag

SEQ ID NO: 70 - DNA, 60 bp, Artificial Sequence
  Synthetic
gtccggacactgtttgtcagcggcctccctgtggacattaaacccagagaactctacttg

SEQ ID NO: 71 - DNA, 60 bp, Artificial Sequence
  Synthetic
tttgaccctgaatttgacctacttgctggggtacagttgcttccttttgaacctccaaca

SEQ ID NO: 72 - DNA, 60 bp, Artificial Sequence
  Synthetic
tggtgacaaaggtgaaacaggtgaacgtggagctgctggcatcaaaggacatcgaggatt

SEQ ID NO: 73 - DNA, 60 bp, Artificial Sequence
  Synthetic
ttaaacacttcttttccttctcttcctcgttttgattgcaccgtttccatctgggggcta

SEQ ID NO: 74 - DNA, 60 bp, Artificial Sequence
  Synthetic
tttggacactgtgctactgaaactcccagccacagcatttatagactgcggtgaacattt

SEQ ID NO: 75 - DNA, 60 bp, Artificial Sequence
  Synthetic
tcttatgtttttagaggcttttccgtaaacatatatcttacatataataaacttttcaaa

SEQ ID NO: 76 - DNA, 60 bp, Artificial Sequence
  Synthetic
tgacggtgacttcaagatcaaatgtgtggcctttgactgaaatcagccagcccatggccc

SEQ ID NO: 77 - DNA, 60 bp, Artificial Sequence
  Synthetic
tgctagtggtttacaaatatgcaactgacaaaagaggatcactttcaggcattggtcctg

SEQ ID NO: 78 - DNA, 60 bp, Artificial Sequence
  Synthetic
tgatcatggagaccgaggcgacagaggtcagaagggccacagaggctttactggtcttca

SEQ ID NO: 79 - DNA, 60 bp, Artificial Sequence
  Synthetic
ttcctgaggatttcagagatggagagtatgaagctgctgttactttagagaagcaggagg

SEQ ID NO: 80 - DNA, 60 bp, Artificial Sequence
  Synthetic
tcgtgacaccaccaaggctgcgggagaagaagtttgaccatcaccctcagcagttcagct

SEQ ID NO: 81 - DNA, 60 bp, Artificial Sequence
  Synthetic
tccgccgggaaacttctgcgggcgccgggctgaagctccgggcagggctgggaaggaaag

SEQ ID NO: 82 - DNA, 60 bp, Artificial Sequence
  Synthetic
acttgatcattctcagtatccactgtctatgtacaataaaggatgtttataagcaaaaaa

SEQ ID NO: 83 - DNA, 60 bp, Artificial Sequence
  Synthetic
tctgttttattactcctggtgcgagtcccgcggactccggcccgctatttgtcatcagct

SEQ ID NO: 84 - DNA, 60 bp, Artificial Sequence
  Synthetic
gactgtcaaaattcattgatgaaaagggtctttgatacctacatgctctttttccaagtc

SEQ ID NO: 85 - DNA, 60 bp, Artificial Sequence
  Synthetic
tgtccccaaactcagcatgactcctgtcctcttcaataaagacgtttctatggccaaaaa

SEQ ID NO: 86 - DNA, 60 bp, Artificial Sequence
  Synthetic
tgtggggaccaaggggacacaatatgagaccaacagcatggacttcaaagttggggcaga

SEQ ID NO: 87 - DNA, 60 bp, Artificial Sequence
  Synthetic
tggtcatcactgacgggcgctcagacactcagagggacaccacaccgctcaacgtgctct

SEQ ID NO: 88 - DNA, 60 bp, Artificial Sequence
  Synthetic
ttgttgtagtaaagtatcttcattagcgttatactccatcatatctggtgtaaactgctc

SEQ ID NO: 89 - DNA, 60 bp, Artificial Sequence
  Synthetic
tttctatgatcagagatattcgagagagggagattcggatctatacggatgcaggccgta

SEQ ID NO: 90 - DNA, 60 bp, Artificial Sequence
  Synthetic
tctccttagacactttggaatctaaccacttaaggacctttttaaagagatagcttctct

SEQ ID NO: 91 - DNA, 60 bp, Artificial Sequence
  Synthetic
ggtcgcttggaccctgatcttacccgtgggcaccctgcgctctgcctgccgcgaagaccg

SEQ ID NO: 92 - DNA, 46 bp, Artificial Sequence
  Synthetic
ctagtgttgtggaggttggtccctgcactcctaatctttttttttt

SEQ ID NO: 93 - DNA, 60 bp, Artificial Sequence
  Synthetic
tttttaactgaaagctgaatccttccatttcttctgcacatctacttgcttaaattgtgg

SEQ ID NO: 94 - DNA, 60 bp, Artificial Sequence
  Synthetic
tttggaattgggagcacgatgactctgagtttgagctattaaagtacttcttacacattg

SEQ ID NO: 95 - DNA, 60 bp, Artificial Sequence
  Synthetic
gagacaattctctattttacagtgtatacagatacaactatttcccctaatagggtggga

SEQ ID NO: 96 - DNA, 60 bp, Artificial Sequence
  Synthetic
tgttgcttagtaacatttatgattttgtgtttctcgtgacagcatgagcagagatcatta

SEQ ID NO: 97 - DNA, 60 bp, Artificial Sequence
  Synthetic
tgcggctgttagggacagaggcaaagaagggcaggacggtccggtttcccgtggatgttc

SEQ ID NO: 98 - DNA, 60 bp, Artificial Sequence
  Synthetic
tgtggtttctacggaatgtatgataagatcctgctttttcgccatgaccctacctctgaa

SEQ ID NO: 99 - DNA, 60 bp, Artificial Sequence
  Synthetic
ttcttcaagggtgcctattacctgaagctggagaaccaaagtctgaagagcgtgaagttt

SEQ ID NO: 100 - DNA, 60 bp, Artificial Sequence
  Synthetic
tagctgtttttcgtcttccctaggctatttctgccgggcgctccgcgaagatgcagctca

SEQ ID NO: 101 - DNA, 60 bp, Artificial Sequence
  Synthetic
tgtcacgtcccctacaagtaaattttgtttctttgaacatttattaaaatgccaagaccc

SEQ ID NO: 102 - DNA, 60 bp, Artificial Sequence
  Synthetic
ataaacatacggattttgttaacgtttatgttaatttcgacaaactggtgatcaccccac

SEQ ID NO: 103 - DNA, 60 bp, Artificial Sequence
  Synthetic
ttgcagtatcaaatctgaatgactctgataagttacagcttctaagtctggtgacaaaaa

SEQ ID NO: 104 - DNA, 60 bp, Artificial Sequence
  Synthetic
ttcctcatcgtctactccgtcactgacaaggccagctttgagcacgtggaccgcttccac

SEQ ID NO: 105 - DNA, 60 bp, Artificial Sequence
  Synthetic
ttgcctccatgcatgtgtgtgtgtgtctgtgaggactggtgtgcgtggacacgtctgaag

SEQ ID NO: 106 - DNA, 60 bp, Artificial Sequence
  Synthetic
gctgcatatttcttttaccattgaccattattatagggacccatgaagtaaatgtcacaa

SEQ ID NO: 107 - DNA, 60 bp, Artificial Sequence
  Synthetic
gtttcatggaagatttgagaaagtgtaaaattattttcataattgtgagaagtatgacaa

SEQ ID NO: 108 - DNA, 60 bp, Artificial Sequence
  Synthetic
ttggcatctatacagtcaaggattgctatcctgtccaggaaacctttaccataaactaca

SEQ ID NO: 109 - DNA, 60 bp, Artificial Sequence
  Synthetic
tgtcttagatgatgagggcagaaacctgaggcagcagaagcttgatcggcagtttttttt

SEQ ID NO: 110 - DNA, 60 bp, Artificial Sequence
  Synthetic
ttctcacccagagacatcaccctgaaatggttcaaaaatgggaatgagctctcagacttc

SEQ ID NO: 111 - DNA, 60 bp, Artificial Sequence
  Synthetic
ttctaagacttactctaagatcttagattctctgtgtctaagattctagatcagatgctc

SEQ ID NO: 112 - DNA, 60 bp, Artificial Sequence
  Synthetic
ttccgaacatctgtgtctttggaacttgccacaacctccctggcctgttccgctgtgagt

SEQ ID NO: 113 - DNA, 60 bp, Artificial Sequence
  Synthetic
ctgaagtagattataaaggtaattctacaaacatgcctgaaacatctcacatcgtagctt

SEQ ID NO: 114 - DNA, 60 bp, Artificial Sequence
  Synthetic
tcggaatgggacaagctcttcatcatgctggagaactcgcagatgagagagcgcatgctg

SEQ ID NO: 115 - DNA, 60 bp, Artificial Sequence
  Synthetic
ttgttgtcttctgattatgtggagattcactacgaaaatgggaaaccacagtactctaag

SEQ ID NO: 116 - DNA, 60 bp, Artificial Sequence
  Synthetic
ccaggatattttcaatattaagtcagtgcatagctgcaccactaacaaattggtgcctgt

SEQ ID NO: 117 - DNA, 60 bp, Artificial Sequence
  Synthetic
ttttcgcaaatgtacagaagccatttgtcacctcagcattcgctgccgaaatgagcaact

SEQ ID NO: 118 - DNA, 60 bp, Artificial Sequence
  Synthetic
ttttaatgtcacctataacaaaatgtgtttggtagcagattgtccagaaagcattttaaa

SEQ ID NO: 119 - DNA, 60 bp, Artificial Sequence
  Synthetic
taggacaaaccattgtaggattttagcaatgtgtatctgtgtgtccctcacaccttttcc

SEQ ID NO: 120 - DNA, 60 bp, Artificial Sequence
  Synthetic
gggggaattactcaattattctatcagaacctattataaagactgtatttcccatagacg

SEQ ID NO: 121 - DNA, 60 bp, Artificial Sequence
  Synthetic
ttctcaaacagctgtcaagtcgccaggatactaaagtgtgaaccatgagaagtatgacaa

SEQ ID NO: 122 - DNA, 60 bp, Artificial Sequence
  Synthetic
ttgcaggaggagatgcttcagagagaggaagccgaaaacaccctgcaatctttcagacag

SEQ ID NO: 123 - DNA, 60 bp, Artificial Sequence
  Synthetic
ttcctgatctgtccacttctggtgtcaaagattttactcatcttcttagtacattctatg

SEQ ID NO: 124 - DNA, 60 bp, Artificial Sequence
  Synthetic
tttaacatgccacatgatgatgcaaagcagtgtgccagccttaatggtgtgaaccaggat

SEQ ID NO: 125 - DNA, 60 bp, Artificial Sequence
  Synthetic
ttactatactttaaagttctatattatgaaaatatataatagcttgtacgcttcaaaaaa

SEQ ID NO: 126 - DNA, 60 bp, Artificial Sequence
  Synthetic
ttccgaggaggctcttactcactcaagaaagtggtgatgatgatccgaccgaaccccaac

SEQ ID NO: 127 - DNA, 60 bp, Artificial Sequence
  Synthetic
tttatgttgctttttagtcgtcagggaaagcttcgactgcaaaaatggtatgtcccacta

SEQ ID NO: 128 - DNA, 60 bp, Artificial Sequence
  Synthetic
tgctgagaaagcctaccatgaacagctttctgtagcagacatcaccaatgcttgctttga

SEQ ID NO: 129 - DNA, 60 bp, Artificial Sequence
  Synthetic
ttttgcagaatcctgctataggaaaaatggagctgttcggtgcatttgtaacgaaaatta

SEQ ID NO: 130 - DNA, 60 bp, Artificial Sequence
  Synthetic
ttgggggatgagatctgctctgcctgtgagtccttccatttcctctgctcctgtgccagt

SEQ ID NO: 131 - DNA, 60 bp, Artificial Sequence
  Synthetic
ttgataacatcagcactgatgacctgaacaccacatcctctgtcagctcttactccaaca

SEQ ID NO: 132 - DNA, 60 bp, Artificial Sequence
  Synthetic
tttttcttactgtcatgtatctgctctcaatatggctgggtaacaagtatatgaagaaca

SEQ ID NO: 133 - DNA, 60 bp, Artificial Sequence
  Synthetic
tatgacaaaggtactctcaaaattcattacaatgctgttcacctgaagatcaaacatcga

SEQ ID NO: 134 - DNA, 60 bp, Artificial Sequence
  Synthetic
tgattgacaagcagatgcccgtcatcatggtcattatgaaggatccttgcttcgccaaat

SEQ ID NO: 135 - DNA, 60 bp, Artificial Sequence
  Synthetic
tttcttaaaacagaaagggtggaaaatcactatacagaagcaatatccaaagatctcctg

SEQ ID NO: 136 - DNA, 60 bp, Artificial Sequence
  Synthetic
tatgaatgatgcattgtttttgcaattgacctatgacaaactgtgaacctgcagatttca

SEQ ID NO: 137 - DNA, 60 bp, Artificial Sequence
  Synthetic
cctgatttttatggaattttaggggatattttgagctttgggttctcagtagtgaattga

SEQ ID NO: 138 - DNA, 60 bp, Artificial Sequence
  Synthetic
ccagttagtattatcatatgtttgtacccgtcacagttttcatagtgctttcaaatacac

SEQ ID NO: 139 - DNA, 60 bp, Artificial Sequence
  Synthetic
tgagcatgacacttctttcagtatattgcttgatgcttcaaataaagttttgtcttggga

SEQ ID NO: 140 - DNA, 60 bp, Artificial Sequence
  Synthetic
tcattgcattcctggtgggtttgatttctatctgcgtgggatctcgaaggcgtttctata

SEQ ID NO: 141 - DNA, 60 bp, Artificial Sequence
  Synthetic
tttggctgaatgcaggcatccattcccgagagtggatctcccaggccactgcaatctgga

SEQ ID NO: 142 - DNA, 60 bp, Artificial Sequence
  Synthetic
tgactgatgttggttgtaatggttgggtttaggatgaaccattttaaggatgccaaatga

SEQ ID NO: 143 - DNA, 60 bp, Artificial Sequence
  Synthetic
gtctcttctcttgtttagttacttacggcaataaatcatctatgagttagtgcaccgtga

SEQ ID NO: 144 - DNA, 60 bp, Artificial Sequence
  Synthetic
ttgatctatgccattcacgccgaggagatcctggagaagcacccgcgagggggcagcttc

SEQ ID NO: 145 - DNA, 46 bp, Artificial Sequence
  Synthetic
gattctgatggggaagagtccgacaggaaccgggcctttttttttt

SEQ ID NO: 146 - DNA, 60 bp, Artificial Sequence
  Synthetic
gctgtgtttttgatactgatattttcctatgctgaatagttttcttactttcagggaagg

SEQ ID NO: 147 - DNA, 60 bp, Artificial Sequence
  Synthetic
gtgtgatatcagcagtcttcagctccttacaaattaccaaaagtggttctaatatgctag

SEQ ID NO: 148 - DNA, 60 bp, Artificial Sequence
  Synthetic
tggggaacctctcacgttgctgtgtcctggtgagcagcccgaccaataaacctgcttttc

SEQ ID NO: 149 - DNA, 60 bp, Artificial Sequence
  Synthetic
ttagttttgtaataccttttttatttgtgaataaaattatcacctggtattcttaaaaaa

SEQ ID NO: 150 - DNA, 60 bp, Artificial Sequence
  Synthetic
ttttgtttaacaaaataattgtaggtttctctctgtaataacaacgctggaaaggccgag

SEQ ID NO: 151 - DNA, 60 bp, Artificial Sequence
  Synthetic
tccatgcttcctactaggatcctgaggctgttggagtttgtggggttttcaggaaacaag

SEQ ID NO: 152 - DNA, 60 bp, Artificial Sequence
  Synthetic
tacaaatgttagagactgtatacgccttcgaggtcttccctatgcagccacaattgagga

SEQ ID NO: 153 - DNA, 60 bp, Artificial Sequence
  Synthetic
ttgccataagaaggtgatgaaggagcgctacgtggaggtggtcccctgttccacagagga

SEQ ID NO: 154 - DNA, 60 bp, Artificial Sequence
  Synthetic
tggggagatgacatcacttgggtacaaacttatgaagaaggtctcttttatgctcaaaaa

SEQ ID NO: 155 - DNA, 60 bp, Artificial Sequence
  Synthetic
tttgtatctcactagcccctcttattttcatatctgccagtgtgctgaggaatggagtgg

SEQ ID NO: 156 - DNA, 60 bp, Artificial Sequence
  Synthetic
ttccaccttactcggctcttctgtggggcgaccctcatcagtgaccgctggctgctcaca

SEQ ID NO: 157 - DNA, 60 bp, Artificial Sequence
  Synthetic
tttagtatgctacttctatgtttattttttgttcttctaataaaatgcataaacttcttg

SEQ ID NO: 158 - DNA, 60 bp, Artificial Sequence
  Synthetic
gctcagaattcatctgaagagagacttaagatgaaagcaaatgattcagctcccttatac

SEQ ID NO: 159 - DNA, 60 bp, Artificial Sequence
  Synthetic
tgtcaaactgtcttggctgtggggctaggggctggggccaaataaagtctcttcctccaa

SEQ ID NO: 160 - DNA, 60 bp, Artificial Sequence
  Synthetic
tcaggagtttgacattgccaggaacgttctagaactgatctatgcacaaactctggtgtg

SEQ ID NO: 161 - DNA, 60 bp, Artificial Sequence
  Synthetic
tccaaagttctcatctatgggaatttgtacgagacctgcttctatctcctgaagaaaact

SEQ ID NO: 162 - DNA, 60 bp, Artificial Sequence
  Synthetic
tcttgtctacaacaagctaactttccagctggaacccaatccccacaccaagtatcagta

SEQ ID NO: 163 - DNA, 60 bp, Artificial Sequence
  Synthetic
tgtacgtctaggcctaggtaaccagtggagtgattatattagcaaatgtgtttgtatcca

SEQ ID NO: 164 - DNA, 60 bp, Artificial Sequence
  Synthetic
tggcacttaaaccttggtagatctgggtttataatcggccattcttaagcacgtggggtt

SEQ ID NO: 165 - DNA, 60 bp, Artificial Sequence
  Synthetic
ttcgaatgaaaagaaatgcatgtttcctgctcttccctcattaaattgcttttaattcca

SEQ ID NO: 166 - DNA, 60 bp, Artificial Sequence
  Synthetic
tatataaactagatagtcctcaaatactgtttgaatttaataaatgtcaatttaaaaatt

SEQ ID NO: 167 - DNA, 60 bp, Artificial Sequence
  Synthetic
gggtaatccggttataatatgtttttcacaggaattaataaatctattttcattttgaat

SEQ ID NO: 168 - DNA, 60 bp, Artificial Sequence
  Synthetic
ttttaatcaagctgcccaaagtcccccaatcactcctggaatacacagagagaggcagca

SEQ ID NO: 169 - DNA, 60 bp, Artificial Sequence
  Synthetic
tgtgattgggttattcaacagcgtaattcagattcatctcctcctgataatgaacaaggc

SEQ ID NO: 170 - DNA, 60 bp, Artificial Sequence
  Synthetic
tggagttgttatgagaattacattagattttgtacgtaaaactcagcatcaagcacacag

SEQ ID NO: 171 - DNA, 60 bp, Artificial Sequence
  Synthetic
tggctgggatctgccacaacatcctggtctgctgccccaaggagctgctggaacagaagg

SEQ ID NO: 172 - DNA, 60 bp, Artificial Sequence
  Synthetic
ttgcactctacctgacacagctgcagcctgcaattcactcccactgcctgggattgcact

SEQ ID NO: 173 - DNA, 60 bp, Artificial Sequence
  Synthetic
ttggtgtgaacttaggtcttggcgtcgggatcccttttcgtcacactcaggtgacctaca

SEQ ID NO: 174 - DNA, 60 bp, Artificial Sequence
  Synthetic
ggacttttttccatatcaaaagaatattttgagtatattggaagctatgatgaagaaatg

SEQ ID NO: 175 - DNA, 60 bp, Artificial Sequence
  Synthetic
ttccctaaatcacaaagcctacagagatgcattcaaaaagatgaagccaccaaaaatccc

SEQ ID NO: 176 - DNA, 60 bp, Artificial Sequence
  Synthetic
ttcttggaatacagcctttcaagcagaggacagaagggtccttctccttatgtgggaaat

SEQ ID NO: 177 - DNA, 60 bp, Artificial Sequence
  Synthetic
ttcaggcagcaagaacaaatcagtagagctggaggatgtaaaattccaccagtgcgtgcg

SEQ ID NO: 178 - DNA, 60 bp, Artificial Sequence
  Synthetic
tacacctacaatggggtggttgcttactccatccatagccaagaaccaaaggacccacac

SEQ ID NO: 179 - DNA, 60 bp, Artificial Sequence
  Synthetic
tggtggaccaccaagtggaggagcataacatcttccacaatgaggtcaaggccatcgggc

SEQ ID NO: 180 - DNA, 60 bp, Artificial Sequence
  Synthetic
ttggacatcttatgaatgtcagaaaataccttttggagggttagaagatcaggggacatg

SEQ ID NO: 181 - DNA, 60 bp, Artificial Sequence
  Synthetic
gttatctgtggaaaacgttttaagttgtcatgtgacagaaacttttcctttgtccatcga

SEQ ID NO: 182 - DNA, 60 bp, Artificial Sequence
  Synthetic
tgcctaccattttacagtatttgtcttctattttggagcctttttattggaagcagcagc

SEQ ID NO: 183 - DNA, 60 bp, Artificial Sequence
  Synthetic
ttgtgacaggatttggagcactgaaaaatgatggttacagtcaaaatcatcttcgacaag

SEQ ID NO: 184 - DNA, 60 bp, Artificial Sequence
  Synthetic
gacttcggaactaaaggagaacttcatccgcttctccaaatctctgggcctccctgaaaa

SEQ ID NO: 185 - DNA, 60 bp, Artificial Sequence
  Synthetic
tgtaccgcattacattatgcctgtgaaatgaaaaaccagtctcttatccctctgctcttg

SEQ ID NO: 186 - DNA, 60 bp, Artificial Sequence
  Synthetic
tctctctagaggtccttttaccttcttcatcataaggatacctatctgaatggtaaaccc

SEQ ID NO: 187 - DNA, 60 bp, Artificial Sequence
  Synthetic
ttgtgctgtaaagagttgctttttgtttatttaatgctgtggcatgggtgaagaggaggg

SEQ ID NO: 188 - DNA, 60 bp, Artificial Sequence
  Synthetic
ttgctacctacccctctggacacttggatatgatcaatggcttctttgaccagttcatag

SEQ ID NO: 189 - DNA, 60 bp, Artificial Sequence
  Synthetic
tttgggtgagatatctttgcacagataacatgtatacatcatagttcaaaacccagtagt

SEQ ID NO: 190 - DNA, 60 bp, Artificial Sequence
  Synthetic
gttcactgctttggtggaaattggtggaaattgctagcaggttccacgatgtttattttt

SEQ ID NO: 191 - DNA, 60 bp, Artificial Sequence
  Synthetic
tgaggtttgactgggacaagtctctgcttaaaatctactctgggtcctcccatcagtggc

SEQ ID NO: 192 - DNA, 60 bp, Artificial Sequence
  Synthetic
tgagaggcgtgaagggcctggagccactctgctagaagagaccaataaagggcaggtgtg

SEQ ID NO: 193 - DNA, 60 bp, Artificial Sequence
  Synthetic
tttttcgggactctgtattccctcttgggctgaccacagcttctccctttcccaaccaat

SEQ ID NO: 194 - DNA, 60 bp, Artificial Sequence
  Synthetic
tcgctcttcaacggcctctcgttccactgcgcgggtgtcctggtggaccagagttgggtg

SEQ ID NO: 195 - DNA, 60 bp, Artificial Sequence
  Synthetic
tgctcaggaattcagtgatgtggagagggccattgagaccctcatcaagaactttcacca

SEQ ID NO: 196 - DNA, 60 bp, Artificial Sequence
  Synthetic
gttggcaattattcccctaggctgagcctgctcatgtacctctgattaataaatgcttat

SEQ ID NO: 197 - DNA, 60 bp, Artificial Sequence
  Synthetic
tggaattcttcctcctctgctgggactcctttgcatggcagggcctcatctcacctctcg

SEQ ID NO: 198 - DNA, 60 bp, Artificial Sequence
  Synthetic
ttggcacatgttctgtgtttcagtaaagagagacctgatcacccatctgtgtgcttccat

SEQ ID NO: 199 - DNA, 60 bp, Artificial Sequence
  Synthetic
tcctgcaataacttcatctatggaggctgccggggcaataagaacagctaccgctctgag

SEQ ID NO: 200 - DNA, 60 bp, Artificial Sequence
  Synthetic
taattttatctttggaagatagctatatggtaactcatcattaaccagaacacctctccc

SEQ ID NO: 201 - DNA, 60 bp, Artificial Sequence
  Synthetic
ttaaaagaagctgaaaatgccaagcgagagggtgaaactagaattcgacgaaatgctgaa

SEQ ID NO: 202 - DNA, 60 bp, Artificial Sequence
  Synthetic
tgtttgatcagtaccagcggagcactgggcaagagctggaggaggctgtccagaaccgtt

SEQ ID NO: 203 - DNA, 60 bp, Artificial Sequence
  Synthetic
ttcggtctctgcggggacgtccacgtgcggctgcgccagcgcatcatcttgtacgaatta

SEQ ID NO: 204 - DNA, 60 bp, Artificial Sequence
  Synthetic
tgctgcactatcgcatcgacaaagacaagacagggaagctctccatccccgagggaaaga

SEQ ID NO: 205 - DNA, 60 bp, Artificial Sequence
  Synthetic
ttgatgagtaaagaaatattgagctctcctccaaatgatgctgttggagaattggagcaa

SEQ ID NO: 206 - DNA, 60 bp, Artificial Sequence
  Synthetic
tggatgtgttcacggatgtggagatcttctgtgacattctagaggcagccaacaagcgtg

SEQ ID NO: 207 - DNA, 60 bp, Artificial Sequence
  Synthetic
ttgagcaggtggctccaaaaggagatgaagaaggtgttcctgctgttgttattgacatgt

SEQ ID NO: 208 - DNA, 60 bp, Artificial Sequence
  Synthetic
tgaggcattattatataaattcaagctcgctcgtgatccttagtaccctgagttgcctga

SEQ ID NO: 209 - DNA, 60 bp, Artificial Sequence
  Synthetic
ggttgcttcaatcagcttttgtatgacatccgaactaatgcagtcaccgtgggtggtgtg

SEQ ID NO: 210 - DNA, 60 bp, Artificial Sequence
  Synthetic
tgcagacgtctggttcaaagagttggatatcaacactgatggtgcagttaacttccagga

SEQ ID NO: 211 - DNA, 60 bp, Artificial Sequence
  Synthetic
ttaaatccaagctgggtttcatcaactgggatgccataaacaaggaccagagaagctctc

SEQ ID NO: 212 - DNA, 60 bp, Artificial Sequence
  Synthetic
ggtggtttttgctgaagacaaaagcagagaagatcagttaaggcattggaagtactggca

SEQ ID NO: 213 - DNA, 60 bp, Artificial Sequence
  Synthetic
ttctggctactttgatgagaggtatgtattgtcctctagagtcagaactggccgaagcat

SEQ ID NO: 214 - DNA, 60 bp, Artificial Sequence
  Synthetic
tagatgtttacaacggactccttcctccctatgcttcttgccacttgacggaattgtact

SEQ ID NO: 215 - DNA, 60 bp, Artificial Sequence
  Synthetic
gtgacttggtagtgatacgctctgtttcttcacttctgcaattgccagacagcatagagg

SEQ ID NO: 216 - DNA, 60 bp, Artificial Sequence
  Synthetic
tggacctggatagcatcatcgctgaggtcaaggcccagtatgaggagattgccaaccgca

SEQ ID NO: 217 - DNA, 60 bp, Artificial Sequence
  Synthetic
tttatattctttggctttgtttattaaaaagcatgattttgctgtgcatgtaccattttg

SEQ ID NO: 218 - DNA, 60 bp, Artificial Sequence
  Synthetic
ttttaaagcagatctctgggacagatggagagggaaacaacgtgccttcaggtgactttt

SEQ ID NO: 219 - DNA, 60 bp, Artificial Sequence
  Synthetic
tcacgggcagttactcggtgtctgagtctcccttcttcagccccatccacctacactcaa

SEQ ID NO: 220 - DNA, 60 bp, Artificial Sequence
  Synthetic
tgaattcattaattacatttctgcaagatggggtggcatgttgtcatcttcagccatttt

SEQ ID NO: 221 - DNA, 60 bp, Artificial Sequence
  Synthetic
ggtgcaacattagaaatttcttgttagtttgcactgagtttattactgtagatagcagac

SEQ ID NO: 222 - DNA, 60 bp, Artificial Sequence
  Synthetic
tttctttgggggtggaaaaggaaaacaattcaagctgagaaaagtattctcaaagatgca

SEQ ID NO: 223 - DNA, 60 bp, Artificial Sequence
  Synthetic
ttgtgaaataaaaacttaaattgtatattttgaaaaataaaacactgaaaagaaaccaac

SEQ ID NO: 224 - DNA, 60 bp, Artificial Sequence
  Synthetic
ttggggaaggatcaagtgaaccatccctagtcttccttcaataaataacttttaactcca

SEQ ID NO: 225 - DNA, 60 bp, Artificial Sequence
  Synthetic
ttttaaaaaatgctatttggaagactatttatttctcgtgtatataatgtatataaaaaa

SEQ ID NO: 226 - DNA, 60 bp, Artificial Sequence
  Synthetic
tgtagagtggtgctgctttaattcataaatcacaaataaaagccaattagctctataact

SEQ ID NO: 227 - DNA, 60 bp, Artificial Sequence
  Synthetic
tttctactgggaattagaatggtgcatacacaatgtattattatcactgtcagatgagca

SEQ ID NO: 228 - DNA, 60 bp, Artificial Sequence
  Synthetic
gtagtacttctgttcactgaagagttatgttacatgaggataaaatggttttgtcgtgtt

SEQ ID NO: 229 - DNA, 60 bp, Artificial Sequence
  Synthetic
ttctcccacctctgtgtgattggactcgtttatggcacagccattatcatgtatgttgga

SEQ ID NO: 230 - DNA, 60 bp, Artificial Sequence
  Synthetic
tttgtttaaatcaacatagcatgaaacaccaaataaaatgtttgacatagttttaaaaaa

SEQ ID NO: 231 - DNA, 60 bp, Artificial Sequence
  Synthetic
ttgtttaccaatgatttatttacaagatatttactcaaataaatggagctgcttacaaaa

SEQ ID NO: 232 - DNA, 60 bp, Artificial Sequence
  Synthetic
tgggtcagttccttattcaagtctgcagccggctcccagggagatctcggtggaacttca

SEQ ID NO: 233 - DNA, 60 bp, Artificial Sequence
  Synthetic
tgccactggtgttaagatatattttgagtggatggaggagaaataaacttattcctcctt

SEQ ID NO: 234 - DNA, 60 bp, Artificial Sequence
  Synthetic
tcagttcaacatgttaaactgaagaaaatgaagtactcttttcagttgtggatcgcaatg

SEQ ID NO: 235 - DNA, 60 bp, Artificial Sequence
  Synthetic
ttgcaccccaatatcatgccctggtgagctatgtccccaagacaatggcacagcctgtgg

SEQ ID NO: 236 - DNA, 60 bp, Artificial Sequence
  Synthetic
ttttgacaaattagagacagagacccattgggtgccagtgagctggttacattcataact

SEQ ID NO: 237 - DNA, 60 bp, Artificial Sequence
  Synthetic
ttcgtaaagccaatgcccctgaaatgctcagtgatggcgaatatatctcagatgttgaag

SEQ ID NO: 238 - DNA, 60 bp, Artificial Sequence
  Synthetic
gttccttgtaaatccaaatgtttctatattgtagctttgcttaaaatggggtcggcccca

SEQ ID NO: 239 - DNA, 60 bp, Artificial Sequence
  Synthetic
ttttcctcatcttgtactggagaaaattcttgtgagtctcactatgaaaaactgtaaagc

SEQ ID NO: 240 - DNA, 60 bp, Artificial Sequence
  Synthetic
tgctgtatgctctgttcggagggtgtgttagtatctccctagaggtgatcttccgggacc

SEQ ID NO: 241 - DNA, 60 bp, Artificial Sequence
  Synthetic
tgaagctgctgtacagcggagtcccattcctgggcccctaccacaaggagtcggctgtga

SEQ ID NO: 242 - DNA, 60 bp, Artificial Sequence
  Synthetic
tttttctcattgacttccttcctgttctaactgccagtactcagaagtcagagttgagag

SEQ ID NO: 243 - DNA, 60 bp, Artificial Sequence
  Synthetic
cagaaatatcctgctttttatttcagagctgacgtttgcaatcctagtgcactagcggaa

SEQ ID NO: 244 - DNA, 60 bp, Artificial Sequence
  Synthetic
ttgaatatcagcgtgctaagtcagaaaaggtgcgaggatgcttacccgagacagatagat

SEQ ID NO: 245 - DNA, 60 bp, Artificial Sequence
  Synthetic
tctggctaaacaatttctgtatggcgaaagaaaaattctaacttgtacgccctcttcatg

SEQ ID NO: 246 - DNA, 60 bp, Artificial Sequence
  Synthetic
ggtatttatgtatcaagatcggacagagtaatatataaatcactccaccgatttggcccc

SEQ ID NO: 247 - DNA, 60 bp, Artificial Sequence
  Synthetic
tttgtaaaatgttctcttatgatcaccatgtattttgtaaataataaaatagtatctgtt

SEQ ID NO: 248 - DNA, 60 bp, Artificial Sequence
  Synthetic
gtaatctcattaccctggactgttctcataatgtaacagatcactaacactgaatcttaa

SEQ ID NO: 249 - DNA, 60 bp, Artificial Sequence
  Synthetic
tgtgtagtttggtgacaagatttgcattcacctggcccaaaccctttttgtctctttggg

SEQ ID NO: 250 - DNA, 60 bp, Artificial Sequence
  Synthetic
acccatgaatgcttccttttctgtaaaatgggacaatgacaggacctgtaaccacacagg

SEQ ID NO: 251 - DNA, 60 bp, Artificial Sequence
  Synthetic
ttcaaagctggagtctgtcctcctaagaaatctgcccagtgccttagatacaagaaacct

SEQ ID NO: 252 - DNA, 60 bp, Artificial Sequence
  Synthetic
tgtgcctcctcaggtatggcagtgactcacctggttttaataaaacaacctgcaacatct

SEQ ID NO: 253 - DNA, 60 bp, Artificial Sequence
  Synthetic
tgacctgctccatggcaaactgtcagtatggctgtgatgttgttaaaggacaaatacggt

SEQ ID NO: 254 - DNA, 60 bp, Artificial Sequence
  Synthetic
tgccactcctgctcagaagacagtggctctgacgtctccagcatctcccaccccacttcg

SEQ ID NO: 255 - DNA, 60 bp, Artificial Sequence
  Synthetic
tggagttggattcttcagatcaccaagtgcattgatgttcatctgaaagaaaatgctgcc

SEQ ID NO: 256 - DNA, 60 bp, Artificial Sequence
  Synthetic
tttttctgtttgtactcttggggaatcacttctttgccatctgttagcaatgcagtcaac

SEQ ID NO: 257 - DNA, 60 bp, Artificial Sequence
  Synthetic
tggcttagttgctctatcatcttacaataagttcaaaaacaactgcttctctgatgccat

SEQ ID NO: 258 - DNA, 60 bp, Artificial Sequence
  Synthetic
gggctttttccctgtcgccttcggcttcctgggcaatgtctgcaacatccccttcctggg

SEQ ID NO: 259 - DNA, 60 bp, Artificial Sequence
  Synthetic
ttttgatgacattgacctgccctcagcagtcaagtacctcatggcttcagaccccaacct

SEQ ID NO: 260 - DNA, 60 bp, Artificial Sequence
  Synthetic
ctcatattacatagcagtatgtttacaaaaggcttataaaaataaaatgaactatcagtt

SEQ ID NO: 261 - DNA, 60 bp, Artificial Sequence
  Synthetic
tttatatttctgggaggaaatgaattcatatctagaagtctggagtgagcaaacaagagc

SEQ ID NO: 262 - DNA, 60 bp, Artificial Sequence
  Synthetic
tgcaactagcaaattcggcttttgccgttgatctgttcaaacaactatgtgaaaaggagc

SEQ ID NO: 263 - DNA, 60 bp, Artificial Sequence
  Synthetic
ttataataaataaaccatgtaagttgaggcttctggtgctataaaggacttttccctcag

SEQ ID NO: 264 - DNA, 60 bp, Artificial Sequence
  Synthetic
tcgcctatatgaaatatggttgcttttgtggcttgggaggccatggccagccccgcgatg

SEQ ID NO: 265 - DNA, 60 bp, Artificial Sequence
  Synthetic
tgtgcctacatcttctatccgcggccccagaacgtggagtactgtgactacagaaagcac

SEQ ID NO: 266 - DNA, 60 bp, Artificial Sequence
  Synthetic
tttcaagaaggctgttcaaaaaatcaaatatcagaaccaggagtgaaagcatcagatcac

SEQ ID NO: 267 - DNA, 60 bp, Artificial Sequence
  Synthetic
tgcgctgtgctctcaataagagcagagaattcaacctgatgtatgatggcaccaaggagg

SEQ ID NO: 268 - DNA, 60 bp, Artificial Sequence
  Synthetic
ttcaaatcaactccagacctccttcgagaccagcaggaggcagccccaccaggcagtgtg

SEQ ID NO: 269 - DNA, 60 bp, Artificial Sequence
  Synthetic
tctgtgcctttctacaactgattgcaacagactgttgagttatgataacaccagtgggaa

SEQ ID NO: 270 - DNA, 60 bp, Artificial Sequence
  Synthetic
ttacaatcgaaacctctatcagtctgcagaggacagctgtggagggttgtattaccatga

SEQ ID NO: 271 - DNA, 46 bp, Artificial Sequence
  Synthetic
aagttgggtttaggacaggtgctgttccgagactcatttttttttt

SEQ ID NO: 272 - DNA, 60 bp, Artificial Sequence
  Synthetic
tgtgcatttataaatgatgtgtattttatatagacctgcttgcattggctgatgctcctc

SEQ ID NO: 273 - DNA, 60 bp, Artificial Sequence
  Synthetic
ttgagtctggtgaggagtctttgcgagagcgaggagcagcggttactggaacaggtgcat

SEQ ID NO: 274 - DNA, 60 bp, Artificial Sequence
  Synthetic
ttttttacaacatcaaaaactttgtccgattccagctgagcacgagcatctccgccctga

SEQ ID NO: 275 - DNA, 60 bp, Artificial Sequence
  Synthetic
caggacgctgtggctgaaatgaaatgattttcaatgtaatcaatgtttaaactggtacta

SEQ ID NO: 276 - DNA, 60 bp, Artificial Sequence
  Synthetic
tgttcacagcaccctcagggtcttaaggtcttcatgccctatcacaaatacctcttttat

SEQ ID NO: 277 - DNA, 60 bp, Artificial Sequence
  Synthetic
ggggtttttcctcttccttctttgtggtttctgttttgtaatttaagaagagctattcat

SEQ ID NO: 278 - DNA, 60 bp, Artificial Sequence
  Synthetic
ggatggtggtttattctcagaagaaaaagatatgtaagtcttttagctcctaagagtgaa

SEQ ID NO: 279 - DNA, 60 bp, Artificial Sequence
  Synthetic
ttctgctgtcctttggagggtgtcttctgggtagagggatgggaaggaagggacccttac

SEQ ID NO: 280 - DNA, 60 bp, Artificial Sequence
  Synthetic
tggagctgcttttaattttagcaaaatgttttatgcaaggcacaataggaagtcagttct

SEQ ID NO: 281 - DNA, 60 bp, Artificial Sequence
  Synthetic
tgagctgcctctaccacagcctcctgcccaccagctggcctcacctcctgaaggcccggg

SEQ ID NO: 282 - DNA, 60 bp, Artificial Sequence
  Synthetic
tttttgaagaagggatgtggctacgatataactttcaggcaccagcaacaaatgccagag

SEQ ID NO: 283 - DNA, 60 bp, Artificial Sequence
  Synthetic
ttctcttttatgtattgagccctgtgttaacatttcacttaagaagagcaccagtgcttt

SEQ ID NO: 284 - DNA, 60 bp, Artificial Sequence
  Synthetic
cggccgggcttggcaggatttccaggcgcgacttggggacgaagccaagttcattcccag

SEQ ID NO: 285 - DNA, 60 bp, Artificial Sequence
  Synthetic
tgggatatcagatctttagtgtgaagatacatctacattaaaccaggaatcactagaact

SEQ ID NO: 286 - DNA, 60 bp, Artificial Sequence
  Synthetic
ttcttttagcattttaattagcagatagcatattatacatactgtttggaactttgcatt

SEQ ID NO: 287 - DNA, 60 bp, Artificial Sequence
  Synthetic
tataatattgtaataatatattttacctgtggtatgtgggcatgtttactgccactggcc

SEQ ID NO: 288 - DNA, 60 bp, Artificial Sequence
  Synthetic
tggcgttctctgcaagccatggtatgctggagcctgtgatcgaaagtctgctgaagaggc

SEQ ID NO: 289 - DNA, 60 bp, Artificial Sequence
  Synthetic
ttcctccttggtcaaccttgactcgttggtcaaggcaccccaggttgcaaagacccggaa

SEQ ID NO: 290 - DNA, 60 bp, Artificial Sequence
  Synthetic
tctcctgtgaatggaggcagagacctccaataaagtgccttctgggctttttctaaaaaa

SEQ ID NO: 291 - DNA, 60 bp, Artificial Sequence
  Synthetic
gcagagtactgtgttgttggtgctgtccatgctgtagctcaaaataaagaagctatttta

SEQ ID NO: 292 - DNA, 60 bp, Artificial Sequence
  Synthetic
tttgatgagtgaatattctggctggcgaactcctacacatccttcaaaacccacctggta

SEQ ID NO: 293 - DNA, 60 bp, Artificial Sequence
  Synthetic
ttatcagggagttacagttacaattgttacagtactgttcccaactcagctgccacgggt

SEQ ID NO: 294 - DNA, 60 bp, Artificial Sequence
  Synthetic
tgcacagtattcttggctgattgatgggaacatccagcaacacacacaagagctctttat

SEQ ID NO: 295 - DNA, 60 bp, Artificial Sequence
  Synthetic
ttgtcaaaattttaaacctttatactcccctgaatgaatttgaagaacgggtaacagtgg

SEQ ID NO: 296 - DNA, 60 bp, Artificial Sequence
  Synthetic
gtacaagaagaacttgaaggccctctacgtggtgcaccccaccagcttcatcaaggtcct

SEQ ID NO: 297 - DNA, 60 bp, Artificial Sequence
  Synthetic
tgtgctttttgggaccatcactgagagtcaggagttttactgcctgtagcaatggccaga

SEQ ID NO: 298 - DNA, 60 bp, Artificial Sequence
  Synthetic
ttcagatctttacatgtataatctcatttctttcatgcagttactctgggatactgttcc

SEQ ID NO: 299 - DNA, 60 bp, Artificial Sequence
  Synthetic
ttcctggggaagcataaccttcggcaaagggagagttcccaggagcagagttctgttgtc

SEQ ID NO: 300 - DNA, 60 bp, Artificial Sequence
  Synthetic
tgcaaagttccctacttcctgtgacttcagctctgttttacaataaaatcttgaaaatgc

SEQ ID NO: 301 - DNA, 60 bp, Artificial Sequence
  Synthetic
taaagaacttcttgcctaaacctgaattaccgcaatttgctgagtgactttgagaaaaat

SEQ ID NO: 302 - DNA, 60 bp, Artificial Sequence
  Synthetic
tgttgggtaacactgagcatcaccccattgattaccccattgccaggcgtgggcacgagt

SEQ ID NO: 303 - DNA, 60 bp, Artificial Sequence
  Synthetic
tatttttctggaaattgaagtgtcaattgggttctcaatatttcatgactccaaggatgc

SEQ ID NO: 304 - DNA, 60 bp, Artificial Sequence
  Synthetic
tgaccttttcactgtgcaaagggagatttctagccaggcattgactattacaatttcatt

SEQ ID NO: 305 - DNA, 60 bp, Artificial Sequence
  Synthetic
gggctgatcttgttggactttaattaatggtatcctttttcacacaccttaaactccaaa

SEQ ID NO: 306 - DNA, 60 bp, Artificial Sequence
  Synthetic
ttgggcattggaaagaagggagttggacccatcgtggagtggaaggcaccgtagctctag

SEQ ID NO: 307 - DNA, 60 bp, Artificial Sequence
  Synthetic
tgtaaaggagtcaaactataaatcaagtatttgggaagtgaagactggaagctaatttgc

SEQ ID NO: 308 - DNA, 60 bp, Artificial Sequence
  Synthetic
ttttgaagaaagcaacaatattgtcaggtttcttgctgtggttctggatgtccagtagca

SEQ ID NO: 309 - DNA, 60 bp, Artificial Sequence
  Synthetic
ttctgagccctcaagaaagatcagaacagattcatgggtgatttagcctatctgtcccag

SEQ ID NO: 310 - DNA, 60 bp, Artificial Sequence
  Synthetic
ttccagttgcctggtgagcgcagctaccatgtctactaccagatcctctcagggaggaag

SEQ ID NO: 311 - DNA, 60 bp, Artificial Sequence
  Synthetic
ttgagtacattcggcgccagaagcaacccaggccacccccaagcagaaggaggaggcccg

SEQ ID NO: 312 - DNA, 60 bp, Artificial Sequence
  Synthetic
tttgactatttgaaactactaggtaaaggcacttttgggaaagttattttggttcgagag

SEQ ID NO: 313 - DNA, 60 bp, Artificial Sequence
  Synthetic
ttgaagatcctctttgtaacttccactccccaaacttcctgaggatctcagaggtggaaa

SEQ ID NO: 314 - DNA, 60 bp, Artificial Sequence
  Synthetic
tgccaataagggccatcttcctgtggtccagatcttgctgaaggctggctgcgaccttga

SEQ ID NO: 315 - DNA, 60 bp, Artificial Sequence
  Synthetic
tgggtaccaactctattgcgcagctcgctgccgtgcgtttaacccaggcgaggaggagga

SEQ ID NO: 316 - DNA, 60 bp, Artificial Sequence
  Synthetic
ttatatgtaattgtataaatggtgcaacagtaataaagttaaacaattaaaaagaaaaaa

SEQ ID NO: 317 - DNA, 60 bp, Artificial Sequence
  Synthetic
tgcagaacaacgacatctccgagctccgcaaggatgacttcaagggtctccagcacctct

SEQ ID NO: 318 - DNA, 60 bp, Artificial Sequence
  Synthetic
ttgttcaggtggggatgtatttcatgtagaaggtggaagaaggctgctatgactctttgg

SEQ ID NO: 319 - DNA, 60 bp, Artificial Sequence
  Synthetic
tgctgttattgaggctgttttggctggcattgcatgttatgctaaaacttccagtctaac

SEQ ID NO: 320 - DNA, 60 bp, Artificial Sequence
  Synthetic
catggcagataggtatcaatatgttttcaatgcctgatgacctataagaagaaagtattg

SEQ ID NO: 321 - DNA, 60 bp, Artificial Sequence
  Synthetic
ctgatctgttaaattcttagtgaagtttccttgatttccagtggctgctgttgtttgagt

SEQ ID NO: 322 - DNA, 60 bp, Artificial Sequence
  Synthetic
ttggttgatcgtgtttttgatgaaagcctcaacttccgaaagattcctccattagttcat

SEQ ID NO: 323 - DNA, 60 bp, Artificial Sequence
  Synthetic
tggatataatcaaaatcatggaagaaagtttgtacagggtaaatctatagacgttgcctg

SEQ ID NO: 324 - DNA, 60 bp, Artificial Sequence
  Synthetic
tttttcagtattatttatagttggcacttgattgcagttctgtgaggcttgagcattcat

SEQ ID NO: 325 - DNA, 60 bp, Artificial Sequence
  Synthetic
gagtgcgcgagaaacagaagctcttccaggaggacaatgacatcccgttgtacctgaagg

SEQ ID NO: 326 - DNA, 60 bp, Artificial Sequence
  Synthetic
tccccagaggaactcaaagttaaggtgttgggagatgtgattgaggtgcatggaaaacat

SEQ ID NO: 327 - DNA, 60 bp, Artificial Sequence
  Synthetic
tggaaacctaactgcaatgtggatgttttacccacatgacttattatgcatgttatgatc

SEQ ID NO: 328 - DNA, 60 bp, Artificial Sequence
  Synthetic
cttggtttatttttcttagaatctgttgctaagactggggacgctgttttcttttacaaa

SEQ ID NO: 329 - DNA, 60 bp, Artificial Sequence
  Synthetic
tgttgggttttagatctcttttcatttgtcaaccttttcagtaaagccctctgttacatc

SEQ ID NO: 330 - DNA, 60 bp, Artificial Sequence
  Synthetic
ggggcatatcattctctgagagaattatttctggatcacaatgacttaaaatctatacca

SEQ ID NO: 331 - DNA, 60 bp, Artificial Sequence
  Synthetic
ggttataaaacagtccagaagtaccccactttcaaatcatgcctgaagaaagaacttcac

SEQ ID NO: 332 - DNA, 60 bp, Artificial Sequence
  Synthetic
ttgggggagattttactcctttcttcaacaactattcactggacaagttctctgctccca

SEQ ID NO: 333 - DNA, 60 bp, Artificial Sequence
  Synthetic
tccatattcagactatatagagaatattctatgcatctatgacgtgcttactactgcagt

SEQ ID NO: 334 - DNA, 60 bp, Artificial Sequence
  Synthetic
tggctcaagttccacattggtatcaaccggtacgagctgtactccagacacaacccggcc

SEQ ID NO: 335 - DNA, 60 bp, Artificial Sequence
  Synthetic
gcgtgctgtatgaatctagaaagccttaatttactaccaagaaataaagcaatatgttcg

SEQ ID NO: 336 - DNA, 60 bp, Artificial Sequence
  Synthetic
ttctctttgccatgaaggaggataatgagaaggtgcctactttgctaacggactacattt

SEQ ID NO: 337 - DNA, 60 bp, Artificial Sequence
  Synthetic
tcggcgccggtaggaagagtcagaggggtgaccagagagcccaacgcctggtgctcaaga

SEQ ID NO: 338 - DNA, 60 bp, Artificial Sequence
  Synthetic
tggaccatgtcaacgattaccatgtcaagcccgagaaggatgcggggtactgctgccact

SEQ ID NO: 339 - DNA, 60 bp, Artificial Sequence
  Synthetic
tcctgtatcttatatggatatatgtatgtgtttgcattgactgggacctctttcacaatt

SEQ ID NO: 340 - DNA, 60 bp, Artificial Sequence
  Synthetic
ttctgttttcatctataaaatcttcaagattgacattgtgctttggtacagggattcctg

SEQ ID NO: 341 - DNA, 60 bp, Artificial Sequence
  Synthetic
ttttggattagagggggatttttgatgggagaaagctggagatctgaacccagcccattt

SEQ ID NO: 342 - DNA, 60 bp, Artificial Sequence
  Synthetic
ttttatagcatgaagatttaatgcctataattaggaaggagttttcacagaaaccctccc

SEQ ID NO: 343 - DNA, 60 bp, Artificial Sequence
  Synthetic
catatgtgaatgttattactctcagtgaattgttattgtttgcaaaaatgcactgggcag

SEQ ID NO: 344 - DNA, 60 bp, Artificial Sequence
  Synthetic
tgaggaaacagagctgacaacacctgtacttccagaagaaacacaggaagaagatgccaa

SEQ ID NO: 345 - DNA, 60 bp, Artificial Sequence
  Synthetic
tttttgaagatgacgccacattaaccctttcattttgtttggaagatggaccaaagcgat

SEQ ID NO: 346 - DNA, 60 bp, Artificial Sequence
  Synthetic
tggacttttggagaatgagttgaaactgatggaagaatttgtcaagcaatataagagcga

SEQ ID NO: 347 - DNA, 60 bp, Artificial Sequence
  Synthetic
ttgccaatgaactggctaaacataccaaagggccagtgtttgctggggatgtaagttctt

SEQ ID NO: 348 - DNA, 60 bp, Artificial Sequence
  Synthetic
ctccaaaatgggcaagagcgaagacttcttctacatcaaggtcagccagaaagcccgggg

SEQ ID NO: 349 - DNA, 60 bp, Artificial Sequence
  Synthetic
ttattatttgcagtcatggagaaccacctacccctgacttctgtttagtctcctttttaa

SEQ ID NO: 350 - DNA, 60 bp, Artificial Sequence
  Synthetic
tgtaaaagagagtcacaggtaccccaaggagtagatgccagggtcctaagttgaaaatga

SEQ ID NO: 351 - DNA, 60 bp, Artificial Sequence
  Synthetic
gttttcacatacaggattaaccttgctgcagtgcgtgtgcaagattaaaaaagatgtcac

SEQ ID NO: 352 - DNA, 60 bp, Artificial Sequence
  Synthetic
tgtccctagctgaactcaggacaacgtgcagcgagaatgagctggctgcggagttcacca

SEQ ID NO: 353 - DNA, 60 bp, Artificial Sequence
  Synthetic
tgcagtgagggtcaaaggagagtcaacatatgtgattgttccataataaacttctggtgt

SEQ ID NO: 354 - DNA, 60 bp, Artificial Sequence
  Synthetic
gtccccaatccttccaaaaatattgatggtgatttgtgctaccatttactcgtttattta

SEQ ID NO: 355 - DNA, 60 bp, Artificial Sequence
  Synthetic
tttattagccttctgaagacagcaaagatgacagtaaaacttaccatccatgctgagaat

SEQ ID NO: 356 - DNA, 60 bp, Artificial Sequence
  Synthetic
tgcatggggaaccatgaactatacatgcgccgtcgcaagcctgataccattgaggtgcag

SEQ ID NO: 357 - DNA, 60 bp, Artificial Sequence
  Synthetic
gctgaccattcactctctgtgtctgttgttggatccaaatacgagtgtttttctttcaaa

SEQ ID NO: 358 - DNA, 60 bp, Artificial Sequence
  Synthetic
gaaaatacaaagaaagttattcagtaccttgcccatgttgcttcttcacataaaggaaga

SEQ ID NO: 359 - DNA, 60 bp, Artificial Sequence
  Synthetic
tttatgaagcacaacacatctcgccagaatgaacactgcctcaccaattttgacctggct

SEQ ID NO: 360 - DNA, 60 bp, Artificial Sequence
  Synthetic
tgtgtgtgatcttgaagggaacagagtcaagggtccagagaaggaggagaagttgagaca

SEQ ID NO: 361 - DNA, 60 bp, Artificial Sequence
  Synthetic
tggatgtttcttatggcatttgctctggggtggagatcatatagacaatcaagtgcaaac

SEQ ID NO: 362 - DNA, 60 bp, Artificial Sequence
  Synthetic
gaaaagcccacggtcatagacagcaccatacaatcaggtatcaaataaaatacgaaatgt

SEQ ID NO: 363 - DNA, 60 bp, Artificial Sequence
  Synthetic
tggacatcccagaaatacatgagagagaaggatatgaagatgaaattgatggtatgacaa

SEQ ID NO: 364 - DNA, 60 bp, Artificial Sequence
  Synthetic
ttctgtctgcagtgtgcacggccttgttctaacccggaataaaggtgattgattgtattg

SEQ ID NO: 365 - DNA, 60 bp, Artificial Sequence
  Synthetic
ttgtttttatttcagttgctgcgaggtctgtcttacatccaccagcgttatattttgcac

SEQ ID NO: 366 - DNA, 60 bp, Artificial Sequence
  Synthetic
tggccacaatattgacacctgttaccatgtatcaatcacagagaagacctgccgaggatt

SEQ ID NO: 367 - DNA, 60 bp, Artificial Sequence
  Synthetic
tggtagctataggaattaacatatacagaacatcaaatgtggaggtgctactacagtttc

SEQ ID NO: 368 - DNA, 60 bp, Artificial Sequence
  Synthetic
tttaagttaatatgaggttctggttcaaggaaaacttacgttggatctgaaccaatgagc

SEQ ID NO: 369 - DNA, 60 bp, Artificial Sequence
  Synthetic
tttcatcgtcattgatatcatgttggacatggccgaaagggaaggggtcgtagacatcta

SEQ ID NO: 370 - DNA, 60 bp, Artificial Sequence
  Synthetic
ggactttgtctgtttattaacctttatatgtttaattaaaataaacaaataaagacaaaa

SEQ ID NO: 371 - DNA, 60 bp, Artificial Sequence
  Synthetic
tttcattcattggctcccatgtactatcgaggctcagctgcagctgttatcgtgtatgat

SEQ ID NO: 372 - DNA, 60 bp, Artificial Sequence
  Synthetic
tgcctgaggaaggaggccgctttgcacgggcacaaagacttccacccccgcgtcacctgc

SEQ ID NO: 373 - DNA, 60 bp, Artificial Sequence
  Synthetic
ttgagtgatgtctcttccccaagatcaataacttcgactccactatcgggaaaggaatcg

SEQ ID NO: 374 - DNA, 60 bp, Artificial Sequence
  Synthetic
tgtgtagatcatcctagaatacctgtgtggtgctgtccttcctcaagactacctcattct

SEQ ID NO: 375 - DNA, 60 bp, Artificial Sequence
  Synthetic
ttttatcctctatccttttttcctttcctaaaagcactctgagtcaagatgagtgggaaa

SEQ ID NO: 376 - DNA, 60 bp, Artificial Sequence
  Synthetic
ttagttaagtgtcttcaaatcttttgggctggtgtggcagcttatgtctgtaatcccagc

SEQ ID NO: 377 - DNA, 60 bp, Artificial Sequence
  Synthetic
tgaagttcctttttagatgtgctattaacattctgttggattcagagggttccttgaaag

SEQ ID NO: 378 - DNA, 60 bp, Artificial Sequence
  Synthetic
tccattgaagaagcttcaggagtgtatcctattgatgacgatgactacgcttctgcgtct

SEQ ID NO: 379 - DNA, 60 bp, Artificial Sequence
  Synthetic
tttgattcaccagactttagcaagatcacaggcaaacccatcaagctgactcaggtggaa

SEQ ID NO: 380 - DNA, 60 bp, Artificial Sequence
  Synthetic
tgatggcataaatggaacagttaactggaagaccagacaggccaacaattttattatcag

SEQ ID NO: 381 - DNA, 60 bp, Artificial Sequence
  Synthetic
ttgcaattcagtgcttggagacagtttttaagatcagcccagaagatacacacctagcag

SEQ ID NO: 382 - DNA, 60 bp, Artificial Sequence
  Synthetic
tttgctttctttgtatgataatcaaattactacagttgcaccaggggcatttgatactct

SEQ ID NO: 383 - DNA, 60 bp, Artificial Sequence
  Synthetic
tttcagagattgttcgtgagttcaagtcgactaaccgcttgctcctaactggaacacctt

SEQ ID NO: 384 - DNA, 60 bp, Artificial Sequence
  Synthetic
ctgtatgaaactgagatgttgtctatagctatgtctataaacaacctgaagacttgtgaa

SEQ ID NO: 385 - DNA, 60 bp, Artificial Sequence
  Synthetic
ttgcaatgtttttattgttaatgtagcatatataaaaaagtatctcataattttaaaaaa

SEQ ID NO: 386 - DNA, 60 bp, Artificial Sequence
  Synthetic
tgtgaaaactgtcaagtgctctgtggatacaagtcgagtgactcttaatggccaataggc

SEQ ID NO: 387 - DNA, 60 bp, Artificial Sequence
  Synthetic
ttttaacagagtcaacctatttgatttcttgacaagaccacaatctgatcccaaagatgt

SEQ ID NO: 388 - DNA, 60 bp, Artificial Sequence
  Synthetic
tgtttatggacagtgctctagatgtgaaaacagatagaactggtttgtgggacaggggca

SEQ ID NO: 389 - DNA, 60 bp, Artificial Sequence
  Synthetic
tgcctgcctggattgcacctttctgccctttccccctcattattaaatgtttctttttgc

SEQ ID NO: 390 - DNA, 60 bp, Artificial Sequence
  Synthetic
gggcttacacgatatcccatacctttaatgcctttggccttccattctgatttctctgat

SEQ ID NO: 391 - DNA, 60 bp, Artificial Sequence
  Synthetic
tttgtatcattcttgagcaatcgctcggtccgtggacaataaacagtattatcaaagaga

SEQ ID NO: 392 - DNA, 60 bp, Artificial Sequence
  Synthetic
ttaccagaagacggatccagccagaagtatgacaagtgtgaaccatgagaagtatgacaa

SEQ ID NO: 393 - DNA, 60 bp, Artificial Sequence
  Synthetic
ttcttacattatgacgtttgttttcaaggagagggtttaaaaatgggatcctgtaagcag

SEQ ID NO: 394 - DNA, 60 bp, Artificial Sequence
  Synthetic
tttcatgattttgctgatcggaaggattgggatgcattccatcctacactggtggcagaa

SEQ ID NO: 395 - DNA, 60 bp, Artificial Sequence
  Synthetic
gtttcttaatggctaccaataaggcaaatatcacaataataaacgccaaattccttaggg

SEQ ID NO: 396 - DNA, 60 bp, Artificial Sequence
  Synthetic
ctggttgttctacttggtaatttgacaccctgttaataacgcaattatttctgtgttctt

SEQ ID NO: 397 - DNA, 60 bp, Artificial Sequence
  Synthetic
tttttgtcctcaagaaaatacggaagaagccctgttgttattgctgattagtgaatcaat

SEQ ID NO: 398 - DNA, 60 bp, Artificial Sequence
  Synthetic
gggcacgagggcagagccagttcctagcgcagagccgcgcccgccatgagggagatcgtg

SEQ ID NO: 399 - DNA, 60 bp, Artificial Sequence
  Synthetic
tgtatagtctttgctatgacttctggccagatgtggaaccatatccgtggacctccatat

SEQ ID NO: 400 - DNA, 60 bp, Artificial Sequence
  Synthetic
tgggtccggctttcggtgactagacggtccgcaggggacatcccgtccctggggcctccc

SEQ ID NO: 401 - DNA, 60 bp, Artificial Sequence
  Synthetic
ttgttggggatcttaaataagattccttttgatctaccggaatatacatgtacagagtac

SEQ ID NO: 402 - DNA, 60 bp, Artificial Sequence
  Synthetic
tcccttatgatttctgctctggctttgcagttttcagcctttcccaagagcagcagaaaa

SEQ ID NO: 403 - DNA, 60 bp, Artificial Sequence
  Synthetic
ttgtgctttttcttttagaagctactaaagggtgttggggatgcttctgactattatgaa

SEQ ID NO: 404 - DNA, 60 bp, Artificial Sequence
  Synthetic
gtaaaactattgtctaacatatatgctttatgtggtcaggaccccttagaattgttgatg

SEQ ID NO: 405 - DNA, 60 bp, Artificial Sequence
  Synthetic
gtggccaaaatcaacagccgatttggataccttcaagacacctgaaaccttatcatgagc

SEQ ID NO: 406 - DNA, 60 bp, Artificial Sequence
  Synthetic
ttcttgctcagaggcctagaagggtacacaaaatgtcttctaaactagaaaacctaaggc

SEQ ID NO: 407 - DNA, 60 bp, Artificial Sequence
  Synthetic
ctgctagtgatagttacctggaacttctaaaagaggctaccaagcgagatctaaatcttt

SEQ ID NO: 408 - DNA, 60 bp, Artificial Sequence
  Synthetic
tttgccaatctctattttgtgggcattgcggttctgaattttatccctgtggtcaatgct

SEQ ID NO: 409 - DNA, 60 bp, Artificial Sequence
  Synthetic
ttggttgtcacttactttttctgtggggaagaaattccataccggaggatgctgaaggct

SEQ ID NO: 410 - DNA, 60 bp, Artificial Sequence
  Synthetic
ttctccaaggagaggcacatcatggacaggacccccgagaaactgaagaaggagctggag

SEQ ID NO: 411 - DNA, 60 bp, Artificial Sequence
  Synthetic
gcctaatacctaggaagatgttgctattcacgttagtaaacagcctaaagaaactcttag

SEQ ID NO: 412 - DNA, 60 bp, Artificial Sequence
  Synthetic
tggcagaagtgaacctttggggcacagtgcggatgacgaaatcctttctccccctcatcc

SEQ ID NO: 413 - DNA, 60 bp, Artificial Sequence
  Synthetic
ttttcttgtttcagaagttcttggccaggctgactgagagatttgtgctgggagtggata

SEQ ID NO: 414 - DNA, 60 bp, Artificial Sequence
  Synthetic
cctcctgacatgagtctgctggaaagagcatccaaacaaacaagtaataaataaataaat

SEQ ID NO: 415 - DNA, 60 bp, Artificial Sequence
  Synthetic
tgtatcaatgacccacatatcaatgacccacgtatcaatgacccgcatatgaatgaaaaa

SEQ ID NO: 416 - DNA, 60 bp, Artificial Sequence
  Synthetic
ccacccagaaatccactcaaatttggggattgtcattccttttgtgaataattaatacaa

SEQ ID NO: 417 - DNA, 60 bp, Artificial Sequence
  Synthetic
gtgatgagtggcgtctttcctgcctctgatgatggactcaataaacagcactggacaagg

SEQ ID NO: 418 - DNA, 60 bp, Artificial Sequence
  Synthetic
tgtttgataccaaaataaatttacgtagagatccttaacttaaaataaattaattttttc

SEQ ID NO: 419 - DNA, 60 bp, Artificial Sequence
  Synthetic
ttcagggaagcagatatcgaacccaatggcaaagtgaagtatgatgaatttatccacaag

SEQ ID NO: 420 - DNA, 60 bp, Artificial Sequence
  Synthetic
tgtgccggtacttcacggacatcatcaagtgccgcgtgatcaacacatcccacctgagca

SEQ ID NO: 421 - DNA, 60 bp, Artificial Sequence
  Synthetic
ttggtactcatgtctcatggcatcctagagggaatctgcggaactgcgcataaaaagaaa

SEQ ID NO: 422 - DNA, 60 bp, Artificial Sequence
  Synthetic
tttttaaaggtttttgaaatccaggaattaaatcatcccttaataaaatattcgaaattc

SEQ ID NO: 423 - DNA, 60 bp, Artificial Sequence
  Synthetic
tgctgctcaagggagccccaagggctggaagggggttgtgaaaccgaaataaactgccaa

SEQ ID NO: 424 - DNA, 60 bp, Artificial Sequence
  Synthetic
tctatcagacagcaattgaaagcgccagacaagctggagacagcgccaagatgcggcgct

SEQ ID NO: 425 - DNA, 60 bp, Artificial Sequence
  Synthetic
gatttaagaatctcctcctacctcctgactcagcaccatgtaatcattaaactctctgct

SEQ ID NO: 426 - DNA, 60 bp, Artificial Sequence
  Synthetic
gcgcgggccggggattaggagacggaggcggactcggagccagggaaccaggggtccggg

SEQ ID NO: 427 - DNA, 60 bp, Artificial Sequence
  Synthetic
ggacaggtgttgtatatagagtggaatctcttggatgcagcttcaagaataaatttttct

SEQ ID NO: 428 - DNA, 60 bp, Artificial Sequence
  Synthetic
catgtcgcgctgggcagggaccggcagccctggaaggggcacttgatatttttcaataaa

SEQ ID NO: 429 - DNA, 60 bp, Artificial Sequence
  Synthetic
ttaataactattgtattaaattgtcatgaaggaacttgtttaataaatggacgtgtaagg

SEQ ID NO: 430 - DNA, 60 bp, Artificial Sequence
  Synthetic
tatttctatattttttaccagaaaaccaaactctccatcgctgaaagagattccagtggg

SEQ ID NO: 431 - DNA, 60 bp, Artificial Sequence
  Synthetic
tgccatttgactcaaacatgaataggacaaagaacagaccgctggttcgtggacagatca

SEQ ID NO: 432 - DNA, 60 bp, Artificial Sequence
  Synthetic
tgggaacctttttagcttggagcttggtgacatatctgcagttcttattactggcttgcc

SEQ ID NO: 433 - DNA, 60 bp, Artificial Sequence
  Synthetic
ttgggcctttaggttccacaatccccatggcttattatccagtcggtcccatctatccac

SEQ ID NO: 434 - DNA, 60 bp, Artificial Sequence
  Synthetic
tttttgtgggcctttccaaaaggacaaatcaacgaggtgctgaaatcttggctgatactt

SEQ ID NO: 435 - DNA, 60 bp, Artificial Sequence
  Synthetic
tcctgcaaaaccatctatggagagaagacggggacccagccccagggaaagatggaggta

SEQ ID NO: 436 - DNA, 60 bp, Artificial Sequence
  Synthetic
ttcttttcctttttcatcgggctctttcctaaaaagctgagctgtaaaatattttacatc

SEQ ID NO: 437 - DNA, 60 bp, Artificial Sequence
  Synthetic
tccactgctcatcgttattctagtgtttttggctctagcagcaagcttcctgctcatctt

SEQ ID NO: 438 - DNA, 60 bp, Artificial Sequence
  Synthetic
ttccaattctagaaggggaagtttttgattctgtgaagccaggactttctgcttttgtag

SEQ ID NO: 439 - DNA, 60 bp, Artificial Sequence
  Synthetic
tttcctcagtaatagtcctgtaaaggtacgtgtttgtcctggctacttgtgctcttcctg

SEQ ID NO: 440 - DNA, 60 bp, Artificial Sequence
  Synthetic
tttttaatgacaatgaagtgacactttgacatttcctaccttttgaggacttgatccttc

SEQ ID NO: 441 - DNA, 60 bp, Artificial Sequence
  Synthetic
agtaaccacctatttattttacctctttcccaaacctggagcatttatgcctaggcttgt

SEQ ID NO: 442 - DNA, 60 bp, Artificial Sequence
  Synthetic
tggtgggcattgagcctctctacatcaaggcagagccggccagccctgacagtccaaagg

SEQ ID NO: 443 - DNA, 60 bp, Artificial Sequence
  Synthetic
tgctcccatccactattaatgcactaggtgggaggagagggcggcaatgacactgcacct

SEQ ID NO: 444 - DNA, 60 bp, Artificial Sequence
  Synthetic
tttcttaaatgacgaagaggccgggaagatccttcaggtgctggaaaggaatgaggagtt

SEQ ID NO: 445 - DNA, 60 bp, Artificial Sequence
  Synthetic
ttactcttcaagttcaaccactgttaagacctcctattgagttttccaggtcctcagatg

SEQ ID NO: 446 - DNA, 60 bp, Artificial Sequence
  Synthetic
taaaaagtacaagtgtggcctcatcaagccctgcccagccaactactttgcgtttaaaat

SEQ ID NO: 447 - DNA, 60 bp, Artificial Sequence
  Synthetic
ttgtcgtttttgttatctaacggtaattacggagtccagaaagagaattggaaatgccgg

SEQ ID NO: 448 - DNA, 60 bp, Artificial Sequence
  Synthetic
gagtgtctgaaattgtggtggtcctgatttataggatttcataattaaaatgtctgctga

SEQ ID NO: 449 - DNA, 60 bp, Artificial Sequence
  Synthetic
ttgctggcgatggcattgagggctcacctgccaaagattttgctctactcacacagtgta

SEQ ID NO: 450 - DNA, 60 bp, Artificial Sequence
  Synthetic
gtcaagatgtcaagtcatttttgaatgtgtctcagggatttctatgctacacattctttt

SEQ ID NO: 451 - DNA, 60 bp, Artificial Sequence
  Synthetic
tgcagagagataatcaccgcaccgtttccagatgtaatactgcaaagaaaaccaatgatg

SEQ ID NO: 452 - DNA, 60 bp, Artificial Sequence
  Synthetic
tggcaaattattatttgttccttgttctcgtgttggacatatctaccgtcttgagggctg

SEQ ID NO: 453 - DNA, 60 bp, Artificial Sequence
  Synthetic
tgccttgtgaagatcattaatgaagtaaagcccacagagatctacaaccttggagcccag

SEQ ID NO: 454 - DNA, 60 bp, Artificial Sequence
  Synthetic
tattgctatcttttctggatgatcagaaaaataattccataaatctattgtctacttgcg

SEQ ID NO: 455 - DNA, 60 bp, Artificial Sequence
  Synthetic
tacataaccagcaagctctcagatgccaactgctgcctggacgccatctgctactactac

SEQ ID NO: 456 - DNA, 60 bp, Artificial Sequence
  Synthetic
gcggcctttgtcacctactgtgataataaagcagtgagtgctgagctctcacccttcccc

SEQ ID NO: 457 - DNA, 60 bp, Artificial Sequence
  Synthetic
cacaaactacctctggacagttgtgttgttttttgttcaatgttccattcttcgacatcc

SEQ ID NO: 458 - DNA, 60 bp, Artificial Sequence
  Synthetic
ttccaggagctgcagatcgatgacaatgagtatgcctacctcaaagccatcatcttcttt

SEQ ID NO: 459 - DNA, 60 bp, Artificial Sequence
  Synthetic
tttctttgggggtgattgtctcgcttgttttcagttgtcgattatatgggagggttctgg

SEQ ID NO: 460 - DNA, 60 bp, Artificial Sequence
  Synthetic
gctactcatggacacattcagctgtgaactccttccctggggggtcaaggtcagcatcat

SEQ ID NO: 461 - DNA, 60 bp, Artificial Sequence
  Synthetic
ctttaacatataaaaaacacttattcccacagagaaaatgtaaaattaaaaatcatcatc

SEQ ID NO: 462 - DNA, 60 bp, Artificial Sequence
  Synthetic
ttcaacataatgaaatagacttgaaagtctctaaggctctatcagttctgacattctagg

SEQ ID NO: 463 - DNA, 60 bp, Artificial Sequence
  Synthetic
ggggttccttctgggcattacatcgcatagaaatcaataatttgtggtgatttggatctg

SEQ ID NO: 464 - DNA, 60 bp, Artificial Sequence
  Synthetic
ttgttcctgaggtgggaggcggtacccgtggctgagaagaaggaggcctgagagcgacat

SEQ ID NO: 465 - DNA, 60 bp, Artificial Sequence
  Synthetic
gcacgctctctctttctctctttaattttggtttctctcaagcttccaaatggtgctcag

SEQ ID NO: 466 - DNA, 60 bp, Artificial Sequence
  Synthetic
gttcttcgtgaactttgtggttgggcaggatccgggctcagacgtcgccttccacttcaa

SEQ ID NO: 467 - DNA, 60 bp, Artificial Sequence
  Synthetic
tgtcaacaagtgtctagatttgaataactgtggattaacaacagcggacatgaaagaaat

SEQ ID NO: 468 - DNA, 60 bp, Artificial Sequence
  Synthetic
tatgtgcctgccatggctgatgaaaacatcattgtacgcaagcagggtaccattttcttg

SEQ ID NO: 469 - DNA, 60 bp, Artificial Sequence
  Synthetic
tttcaccaagagagtgtttcttcactcaactcaggtggcatttggggtgacatctcaaag

SEQ ID NO: 470 - DNA, 60 bp, Artificial Sequence
  Synthetic
cggaaagaacacccggaaatgaaaggccaccaagaagaaagaccatgagaagtatgacaa

SEQ ID NO: 471 - DNA, 60 bp, Artificial Sequence
  Synthetic
ctttggggacaggaagtcggcacatctccaggtcttcatgtgcacaatatagagtttatt

SEQ ID NO: 472 - DNA, 60 bp, Artificial Sequence
  Synthetic
ttctctccatgaattttcagggagctaggggtgtcagtatcccgccatgtagcatttccc

SEQ ID NO: 473 - DNA, 60 bp, Artificial Sequence
  Synthetic
tgtgtgcaaatgagtgcacacacacagaaggggtccagaggggagaatgccaccaacaga

SEQ ID NO: 474 - DNA, 60 bp, Artificial Sequence
  Synthetic
gtatgtatcacccaactcactaattatcaacttatgtgctatcagatatcctctctaccc

SEQ ID NO: 475 - DNA, 60 bp, Artificial Sequence
  Synthetic
tgaacggcatgctgattcgtgaggcccggagctacatcttgcgctgccatggctgtttca

SEQ ID NO: 476 - DNA, 60 bp, Artificial Sequence
  Synthetic
ttttcctggttcaacaacctgttgacttccctggaacaggagatggaggaattaggcaaa

SEQ ID NO: 477 - DNA, 60 bp, Artificial Sequence
  Synthetic
tgtagccctgggtttaatgtcaaatcaaggcaaaaggaattaaataatgtacttttggct

SEQ ID NO: 478 - DNA, 60 bp, Artificial Sequence
  Synthetic
tgaagttttctttattacggattcaaagacttattttgaaagttggaaggagaagggagg

SEQ ID NO: 479 - DNA, 60 bp, Artificial Sequence
  Synthetic
tggctgctctgatctggtctcagcgcggagggagcagagggagtccatggaggatccctc

SEQ ID NO: 480 - DNA, 60 bp, Artificial Sequence
  Synthetic
ttttgtgttttttaaggcattaataaagccttcgataatattaaatacaaaatgaaaaaa

SEQ ID NO: 481 - DNA, 60 bp, Artificial Sequence
  Synthetic
gtaagtcacatttctattaggactacttacaaggacaaggtttccatttttccagttgta

SEQ ID NO: 482 - DNA, 60 bp, Artificial Sequence
  Synthetic
tgtggctatcttatgtgaggactagaggtgaagaggagatggacactgcctctggagcca

SEQ ID NO: 483 - DNA, 60 bp, Artificial Sequence
  Synthetic
ttgcagatgctttagtgatctttcagctctatgagatgatccgagtgccagtcaactgga

SEQ ID NO: 484 - DNA, 60 bp, Artificial Sequence
  Synthetic
ttccttgtcgaatgatactgtaatgaccttccaaagtgaagagtagcacattaaagtgat

SEQ ID NO: 485 - DNA, 60 bp, Artificial Sequence
  Synthetic
tcattgattcctaaaggattatcagagtttacaaaacagcaaatacgctacattctgcag

SEQ ID NO: 486 - DNA, 60 bp, Artificial Sequence
  Synthetic
tgcgttttgtgctttgatgccaggaatgccgcctagtttatgtccccggtggggcacaca

SEQ ID NO: 487 - DNA, 60 bp, Artificial Sequence
  Synthetic
tatttttacaatacaggtttgcagaaccagcgagtattatatgtacaggattccttagag

SEQ ID NO: 488 - DNA, 60 bp, Artificial Sequence
  Synthetic
gggaagctcctgttcctcttattccaatgttcttggtttttctttatataacttgatgaa

SEQ ID NO: 489 - DNA, 60 bp, Artificial Sequence
  Synthetic
ttgcttgttgctcactgtgctgcttttccatgagctcttggaggcaccaagaaataaact

SEQ ID NO: 490 - DNA, 60 bp, Artificial Sequence
  Synthetic
ttctgcgcggggtgtgaggtggatactttgaacattctgagaacccaataaaactagaag

SEQ ID NO: 491 - DNA, 60 bp, Artificial Sequence
  Synthetic
tgtactacgagtccaaggcacatatccactgctcggtgaaagcagagaactcggtggcgg

SEQ ID NO: 492 - DNA, 60 bp, Artificial Sequence
  Synthetic
ttttgacatacaatatggagtagtggttattcgcctaaaagaaggtctggatatatctca

SEQ ID NO: 493 - DNA, 60 bp, Artificial Sequence
  Synthetic
ttgtaggaaatttgcatacccgtaaagggagacttttttaaataacagttgagtctttgc

SEQ ID NO: 494 - DNA, 60 bp, Artificial Sequence
  Synthetic
ttcctcctcttcaacatcatggactggctgggacggagcctgacctcttacttcctgtgg

SEQ ID NO: 495 - DNA, 60 bp, Artificial Sequence
  Synthetic
tttgaactatcaagcatatactgtatacagttagaaagttattaaatgaacattttactc

SEQ ID NO: 496 - DNA, 60 bp, Artificial Sequence
  Synthetic
tgatccgcctcatacacaaggagctgagctgcccagggtcagctacgggggaccaagttc

SEQ ID NO: 497 - DNA, 60 bp, Artificial Sequence
  Synthetic
tggtgctcatcttcctgcggcagcggattcgtattgccatcgccctcctgaaggaggcca

SEQ ID NO: 498 - DNA, 60 bp, Artificial Sequence
  Synthetic
tgtggtttataatccttgaatattgttttagaaactttggtctccctggttcctgccact

SEQ ID NO: 499 - DNA, 60 bp, Artificial Sequence
  Synthetic
ttgtttgatcatgtgaagactggaattgaagatgtttgtggacattggggtcacaacttt

SEQ ID NO: 500 - DNA, 60 bp, Artificial Sequence
  Synthetic
ttttaaataagaaatgctaacgtttactgttactgctgtgtgctatgcactttgctaagc

SEQ ID NO: 501 - DNA, 60 bp, Artificial Sequence
  Synthetic
ttttggtagatttttatctatacaaatttaaataaaattatgttttgtaagctgaaaaaa

SEQ ID NO: 502 - DNA, 60 bp, Artificial Sequence
  Synthetic
tgtctgtagttgattgaaacgagggcagttatgaattgatttgggcaatcaaatgaattt

SEQ ID NO: 503 - DNA, 60 bp, Artificial Sequence
  Synthetic
ttttctggaaaatggtgcagtcccgcgtgggtgactccttctacatccgcactcactttg

SEQ ID NO: 504 - DNA, 60 bp, Artificial Sequence
  Synthetic
tggcggagaacgtgtttgctgtacgctgtgctcagctcacccaccagctgctggagctga

SEQ ID NO: 505 - DNA, 60 bp, Artificial Sequence
  Synthetic
ttgcaagaatgaaatgaatgattctacagctaggacttaaccttgaaatggaaagtcttg

SEQ ID NO: 506 - DNA, 60 bp, Artificial Sequence
  Synthetic
ggccccgcctcctttctgttttatttttgaggaaataaaataaccaagtgctaaatcttg

SEQ ID NO: 507 - DNA, 60 bp, Artificial Sequence
  Synthetic
ggtaaaggttattcctttcctttcctggagctacacctttctttgtaaaactgtactgtg

SEQ ID NO: 508 - DNA, 60 bp, Artificial Sequence
  Synthetic
tgcatctactttcagtcaggcaacaatgaagagccttatgtcagtatcaccaagaagagg

SEQ ID NO: 509 - RNA, 21 nt, Homo sapiens
uaacagucuccagucacggcc

SEQ ID NO: 510 - RNA, 22 nt, Homo sapiens
acagcaggcacagacaggcagu

SEQ ID NO: 511 - RNA, 22 nt, Homo sapiens
uaacagucuacagccauggucg

SEQ ID NO: 512 - RNA, 21 nt, Homo sapiens
uccgguucucagggcuccacc

SEQ ID NO: 513 - RNA, 22 nt, Homo sapiens
aacccguagauccgaucuugug

SEQ ID NO: 514 - RNA, 22 nt, Homo sapiens
aacccguagauccgaacuugug

SEQ ID NO: 515 - RNA, 22 nt, Homo sapiens
aacuggcccucaaagucccgcu

SEQ ID NO: 516 - RNA, 22 nt, Homo sapiens
ggagaaauuauccuuggugugu

SEQ ID NO: 517 - RNA, 22 nt, Homo sapiens
uggcucaguucagcaggaacag

SEQ ID NO: 518 - RNA, 22 nt, Homo sapiens
gugacaucacauauacggcagc

SEQ ID NO: 519 - RNA, 22 nt, Homo sapiens
acggguuaggcucuugggagcu

SEQ ID NO: 520 - RNA, 22 nt, Homo sapiens
aucaugaugggcuccucggugu

SEQ ID NO: 521 - RNA, 23 nt, Homo sapiens
ucuuggaguaggucauugggugg

SEQ ID NO: 522 - RNA, 23 nt, Homo sapiens
ucucacacagaaaucgcacccgu

SEQ ID NO: 523 - RNA, 21 nt, Homo sapiens
uaaggcacccuucugaguaga

SEQ ID NO: 524 - RNA, 22 nt, Homo sapiens
ucuacagugcacgugucuccag

SEQ ID NO: 525 - RNA, 23 nt, Homo sapiens
ucggggaucaucaugucacgaga

SEQ ID NO: 526 - RNA, 22 nt, Homo sapiens
ucccugagacccuaacuuguga

SEQ ID NO: 527 - RNA, 22 nt, Homo sapiens
ugaaggucuacugugugccagg

SEQ ID NO: 528 - RNA, 22 nt, Homo sapiens
cacccguagaaccgaccuugcg

SEQ ID NO: 529 - RNA, 22 nt, Homo sapiens
aacuggccuacaaagucccagu

SEQ ID NO: 530 - RNA, 22 nt, Homo sapiens
aacccguagauccgaucuugug

SEQ ID NO: 531 - RNA, 22 nt, Homo sapiens
cuuucagucggauguuugcagc

SEQ ID NO: 532 - RNA, 23 nt, Homo sapiens
ucuuugguuaucuagcuguauga

SEQ ID NO: 533 - RNA, 22 nt, Homo sapiens
cacuggcuccuuucuggguaga

SEQ ID NO: 534 - RNA, 21 nt, Homo sapiens
uacucaaaaagcugucaguca

SEQ ID NO: 535 - RNA, 22 nt, Homo sapiens
uaaugccccuaaaaauccuuau

SEQ ID NO: 536 - RNA, 21 nt, Homo sapiens
ucagugcaugacagaacuugg

SEQ ID NO: 537 - RNA, 22 nt, Homo sapiens
ugagguaguagguuguaugguu

SEQ ID NO: 538 - RNA, 22 nt, Homo sapiens
ucucccaacccuuguaccagug

SEQ ID NO: 539 - RNA, 22 nt, Homo sapiens
aaugcaccugggcaaggauuca

SEQ ID NO: 540 - RNA, 22 nt, Homo sapiens
cagugguuuuacccuaugguag

SEQ ID NO: 541 - RNA, 22 nt, Homo sapiens
ugggucuuugcgggcgagauga

SEQ ID NO: 542 - RNA, 22 nt, Homo sapiens
cgggguuuugagggcgagauga

SEQ ID NO: 543 - RNA, 21 nt, Homo sapiens
aggcggagacuugggcaauug

SEQ ID NO: 544 - RNA, 22 nt, Homo sapiens
uggugggcacagaaucuggacu

SEQ ID NO: 545 - RNA, 22 nt, Homo sapiens
ugugacugguugaccagagggg

SEQ ID NO: 546 - RNA, 22 nt, Homo sapiens
aacccguagauccgaucuugug

SEQ ID NO: 547 - RNA, 21 nt, Homo sapiens
caucccuugcaugguggaggg

SEQ ID NO: 548 - RNA, 21 nt, Homo sapiens
agcuacaucuggcuacugggu

SEQ ID NO: 549 - RNA, 22 nt, Homo sapiens
cuuucagucggauguuuacagc

SEQ ID NO: 550 - RNA, 24 nt, Homo sapiens
ucccugagacccuuuaaccuguga

SEQ ID NO: 551 - RNA, 21 nt, Homo sapiens
aaagugcuuccuuuuugaggg

SEQ ID NO: 552 - RNA, 22 nt, Homo sapiens
acaguagucugcacauugguua

SEQ ID NO: 553 - RNA, 22 nt, Homo sapiens
cugaagcucagagggcucugau

SEQ ID NO: 554 - RNA, 21 nt, Homo sapiens
aauauaacacagauggccugu

SEQ ID NO: 555 - RNA, 22 nt, Homo sapiens
ucguaccgugaguaauaaugcg

SEQ ID NO: 556 - RNA, 22 nt, Homo sapiens
augcaccugggcaaggauucug

SEQ ID NO: 557 - RNA, 23 nt, Homo sapiens
uagcagcgggaacaguucugcag

SEQ ID NO: 558 - RNA, 28 nt, Homo sapiens
ucacaaugcugacacucaaacugcugac

SEQ ID NO: 559 - RNA, 22 nt, Homo sapiens
augcugacauauuuacuagagg

SEQ ID NO: 560 - RNA, 22 nt, Homo sapiens
ugagaacugaauuccauaggcu

SEQ ID NO: 561 - RNA, 21 nt, Homo sapiens
gcaguccaugggcauauacac

SEQ ID NO: 562 - RNA, 22 nt, Homo sapiens
cacgcucaugcacacacccaca

SEQ ID NO: 563 - RNA, 22 nt, Homo sapiens
cacccguagaaccgaccuugcg

SEQ ID NO: 564 - RNA, 22 nt, Homo sapiens
gaauguugcucggugaaccccu

SEQ ID NO: 565 - RNA, 23 nt, Homo sapiens
guccaguuuucccaggaaucccu

SEQ ID NO: 566 - RNA, 22 nt, Homo sapiens
gguccagaggggagauagguuc

SEQ ID NO: 567 - RNA, 23 nt, Homo sapiens
cacccggcugugugcacaugugc

SEQ ID NO: 568 - RNA, 22 nt, Homo sapiens
caaucagcaaguauacugcccu

SEQ ID NO: 569 - RNA, 21 nt, Homo sapiens
ugguagacuauggaacguagg

SEQ ID NO: 570 - RNA, 21 nt, Homo sapiens
uagcagcacagaaauauuggc

SEQ ID NO: 571 - RNA, 22 nt, Homo sapiens
acaggugagguucuugggagcc

SEQ ID NO: 572 - RNA, 22 nt, Homo sapiens
ucggauccgucugagcuuggcu

SEQ ID NO: 573 - RNA, 21 nt, Homo sapiens
uaccacaggguagaaccacgg

SEQ ID NO: 574 - RNA, 22 nt, Homo sapiens
aagacgggaggaaagaagggag

SEQ ID NO: 575 - RNA, 21 nt, Homo sapiens
caaaacgugaggcgcugcuau

SEQ ID NO: 576 - RNA, 21 nt, Homo sapiens
gccccugggccuauccuagaa

SEQ ID NO: 577 - RNA, 19 nt, Homo sapiens
aggcugcggaauucaggac

SEQ ID NO: 578 - RNA, 24 nt, Homo sapiens
acaaagugcuucccuuuagagugu

SEQ ID NO: 579 - RNA, 20 nt, Homo sapiens
guagaggagauggcgcaggg

SEQ ID NO: 580 - RNA, 25 nt, Homo sapiens
cuagugagggacagaaccaggauuc

SEQ ID NO: 581 - RNA, 23 nt, Homo sapiens
cccaguguuuagacuaucuguuc

SEQ ID NO: 582 - RNA, 22 nt, Homo sapiens
aaggagcucacagucuauugag

SEQ ID NO: 583 - RNA, 21 nt, Homo sapiens
gaaagcgcuucucuuuagagg

SEQ ID NO: 584 - RNA, 22 nt, Homo sapiens
aaauuauuguacaucggaugag

SEQ ID NO: 585 - RNA, 22 nt, Homo sapiens
uagcagcacauaaugguuugug

SEQ ID NO: 586 - RNA, 22 nt, Homo sapiens
uuuguucguucggcucgcguga

SEQ ID NO: 587 - RNA, 22 nt, Homo sapiens
cuauacagucuacugucuuucc

SEQ ID NO: 588 - RNA, 52 nt, Homo sapiens
gaacuuauugacgggcggacagaaacugugugcugauugucacguucugauu

SEQ ID NO: 589 - RNA, 22 nt, Homo sapiens
auguagggcuaaaagccauggg

SEQ ID NO: 590 - RNA, 22 nt, Homo sapiens
acugcauuaugagcacuuaaag

SEQ ID NO: 591 - RNA, 22 nt, Homo sapiens
cugugcgugugacagcggcuga

SEQ ID NO: 592 - RNA, 23 nt, Homo sapiens
aguuuugcagguuugcauccagc

SEQ ID NO: 593 - RNA, 21 nt, Homo sapiens
uggguuuacguugggagaacu

SEQ ID NO: 594 - RNA, 21 nt, Homo sapiens
uacaguacugugauaacugaa

SEQ ID NO: 595 - RNA, 24 nt, Homo sapiens
gauugcucugcgugcggaaucgac

SEQ ID NO: 596 - RNA, 22 nt, Homo sapiens
uggcaguguauuguuagcuggu

SEQ ID NO: 597 - RNA, 22 nt, Homo sapiens
aucgugcauccuuuuagagugu

SEQ ID NO: 598 - RNA, 22 nt, Homo sapiens
accaucgaccguugauuguacc

SEQ ID NO: 599 - RNA, 21 nt, Homo sapiens
uacugcagacaguggcaauca

SEQ ID NO: 600 - RNA, 22 nt, Homo sapiens
ugugucacucgaugaccacugu

SEQ ID NO: 601 - RNA, 23 nt, Homo sapiens
uagcaccauuugaaaucaguguu

SEQ ID NO: 602 - RNA, 23 nt, Homo sapiens
uaaggugcaucuagugcaguuag

SEQ ID NO: 603 - RNA, 60 nt, Homo sapiens
ccuggaugaugauagcaaaugcugacugaacaugaaggucuuaauuagcucuaacugacu

SEQ ID NO: 604 - RNA, 22 nt, Homo sapiens
aaaccuguguuguucaagaguc

SEQ ID NO: 605 - RNA, 22 nt, Homo sapiens
cagugcaaugaugaaagggcau

SEQ ID NO: 606 - RNA, 23 nt, Homo sapiens
uggaagacuagugauuuuguugu

SEQ ID NO: 607 - RNA, 22 nt, Homo sapiens
cuuucagucagauguuugcugc

SEQ ID NO: 608 - RNA, 23 nt, Homo sapiens
uaauacugccggguaaugaugga

SEQ ID NO: 609 - RNA, 22 nt, Homo sapiens
aaagugcauccuuuuagagugu

SEQ ID NO: 610 - RNA, 22 nt, Homo sapiens
ccgcacuguggguacuugcugc

SEQ ID NO: 611 - RNA, 23 nt, Homo sapiens
gcagcagagaauaggacuacguc

SEQ ID NO: 612 - RNA, 19 nt, Homo sapiens
ucuaggcugguacugcuga

SEQ ID NO: 613 - RNA, 22 nt, Homo sapiens
cgaaucauuauuugcugcucua

SEQ ID NO: 614 - RNA, 22 nt, Homo sapiens
aagugcugucauagcugagguc

SEQ ID NO: 615 - RNA, 23 nt, Homo sapiens
agugccugagggaguaagagccc

SEQ ID NO: 616 - RNA, 21 nt, Homo sapiens
aggcaagaugcuggcauagcu

SEQ ID NO: 617 - RNA, 22 nt, Homo sapiens
ccuauucuugauuacuuguuuc

SEQ ID NO: 618 - RNA, 22 nt, Homo sapiens
ucagugcacuacagaacuuugu

SEQ ID NO: 619 - RNA, 23 nt, Homo sapiens
aaugacacgaucacucccguuga

SEQ ID NO: 620 - RNA, 22 nt, Homo sapiens
ucagugcaucacagaacuuugu

SEQ ID NO: 621 - RNA, 22 nt, Homo sapiens
uaauacugccugguaaugauga

SEQ ID NO: 622 - RNA, 22 nt, Homo sapiens
aggcaguguauuguuagcuggc

SEQ ID NO: 623 - RNA, 22 nt, Homo sapiens
gaaaucaagcgugggugagacc

SEQ ID NO: 624 - RNA, 22 nt, Homo sapiens
uaacacugucugguaaagaugg

SEQ ID NO: 625 - RNA, 20 nt, Homo sapiens
guguguggaaaugcuucugc

SEQ ID NO: 626 - RNA, 22 nt, Homo sapiens
caucuuccaguacaguguugga

SEQ ID NO: 627 - RNA, 22 nt, Homo sapiens
cuguugccacuaaccucaaccu

SEQ ID NO: 628 - RNA, 22 nt, Homo sapiens
uaauacugucugguaaaaccgu

SEQ ID NO: 629 - RNA, 22 nt, Homo sapiens
ccaguauuaacugugcugcuga

SEQ ID NO: 630 - RNA, 22 nt, Homo sapiens
caucuuaccggacagugcugga

SEQ ID NO: 631 - RNA, 22 nt, Homo sapiens
uauaccucaguuuuaucaggug

SEQ ID NO: 632 - RNA, 22 nt, Homo sapiens
gugugcggaaaugcuucugcua

SEQ ID NO: 633 - RNA, 22 nt, Homo sapiens
ucuucucuguuuuggccaugug

SEQ ID NO: 634 - RNA, 22 nt, Homo sapiens
uccauuacacuacccugccucu

SEQ ID NO: 635 - RNA, 22 nt, Homo sapiens
caucuuacugggcagcauugga

SEQ ID NO: 636 - RNA, 22 nt, Homo sapiens
aucgugcaucccuuuagagugu

SEQ ID NO: 637 - RNA, 22 nt, Homo sapiens
aagauguggaaaaauuggaauc

SEQ ID NO: 638 - RNA, 22 nt, Homo sapiens
caauguuuccacagugcaucac

SEQ ID NO: 639 - RNA, 22 nt, Homo sapiens
uaacacugucugguaacgaugu

(19) United States
(12) Patent Application Publication
(10) Pub. No.: US 2014/0031251 A1
(43) Pub. Date: Jan. 30, 2014

(54) METHODS OF CLASSIFYING HUMAN SUBJECTS WITH REGARD TO CANCER PROGNOSIS

(71) Applicants: Timothy Yeatman, Greenville, SC (US); Andrey Loboda, Philadelphia, PA (US); Michael Nebozhyn, Yeadon, PA (US); Hongyue Dai, Chestnut Hill, MA (US)

(72) Inventors: Timothy Yeatman, Greenville, SC (US); Andrey Loboda, Philadelphia, PA (US); Michael Nebozhyn, Yeadon, PA (US); Hongyue Dai, Chestnut Hill, MA (US)

(73) Assignees: H. Lee Moffitt Cancer Center and Research Institute, Inc., Tampa, FL (US); Merck Sharp & Dohme Corp., Rahway, NJ (US)

(21) Appl. No.: 13/883,478

(22) PCT Filed: Nov. 2, 2011
(86) PCT No.: PCT/US11/58990

§ 371 Date: Oct. 8, 2013

Related U.S. Application Data
(60) Provisional application No. 61/409,840, filed on Nov. 3, 2010.

(51) Int. Cl. C12Q 1/68

(52) U.S. Cl. CPC C12Q 1/6886
USPC 506/9

(57) ABSTRACT

In one aspect, methods, markers, and expression signatures are disclosed for assessing the degree to which a cell sample has epithelial cell-like properties or mesenchymal cell-like properties. In another aspect, methods are provided for predicting cancer patient prognosis based on whether the cancer is classified as having a high or low EMT Signature Score.

### CROSS-REFERENCE TO RELATED APPLICATION

This application claims the benefit of Provisional Application No. 61/409,840, filed Nov. 3, 2010, the disclosure of which is incorporated herein by reference.

### STATEMENT REGARDING SEQUENCE LISTING

The sequence listing associated with this application is provided in text format in lieu of a paper copy and is hereby incorporated by reference into the specification. The name of the text file containing the sequence listing is: 38156_Seq_Final_{—}2011-11-02.txt. The file is 111 KB; was created on Nov. 2, 2011; and is being submitted via EFS-Web with the filing of the specification

### FIELD OF THE INVENTION

The invention relates generally to the use of gene expression marker gene sets that are correlated to the epithelial cell to mesenchymal cell transition (EMT) to predict cancer progression, cancer recurrence and cancer prognosis. One aspect of the invention relates to the use of the EMT Signature or another selected set of gene markers, referred to as the PC1 Signature, which is also related to EMT, to evaluate or compare tumor samples obtained from a mammalian subject and predict the subject's response to cancer therapy agents, cancer progression, cancer recurrence, and to predict a subject's cancer prognosis. Yet another aspect of the invention relates to the use of an miRNA or a plurality of miRNAs, whose expression levels are shown to correlate with the EMT Signature and PC1 Signature scores (“MicroRNA Signature markers”), to predict cancer progression, cancer recurrence and cancer prognosis in a cancer patient.

### BACKGROUND

Changes in cell phenotype between epithelial and mesenchymal states, defined as epithelial-mesenchymal (EMT) and mesenchymal-epithelial (MET) transitions, have key roles in embryonic development, and their importance in the pathogenesis of cancer and other human diseases is recognized (Polyak et al., 2009, Nature Rev., 272:265-73; Baum et al., 2008, Semin. Cell Dev. Biol. 19:294-308; Hugo et al., 2007, J. Cell Physiol. 213:374-83).

The term EMT refers to a complex molecular and cellular program by which epithelial cells shed their differentiated characteristics, including cell-cell adhesion, planar and apical-basal polarity, and lack of motility, and acquire instead mesenchymal cell-like features, including motility, invasiveness and a heightened resistance to apoptosis. Thus, similar to embryonic development, both EMT and MET seem to have crucial roles in the tumorigenic process. In particular, EMT has been found to contribute to invasion, metastatic dissemination and acquisition of therapeutic resistance. In contrast, MET—the reversal of EMT—seems to occur following cancer dissemination and the subsequent formation of distant metastases (Polyak et al., 2009, Nature Rev. 272:265-73) Importantly, initiation of the EMT program has been associated with poor clinical outcome in multiple tumor types (Sabbah et al., 2008, Drug Resist. Updat. 11:123-51), most likely because of the aggressive cell-biological traits that this program confers on carcinoma cells within primary tumors.

The identification of patient subpopulations most likely to respond to therapy is a central goal of modern molecular medicine. This notion is particularly important for cancer due to the large number of approved and experimental therapies (Rothenberg et al., 2003, Nat. Rev. Cancer 3:303-309), low response rates to many current treatments, and clinical importance of using the optimal therapy in the first treatment cycle (Dracopoli, 2005, Curr. Mol. Med. 5:103-110). In addition, the narrow therapeutic index and severe toxicity profiles associated with currently marketed cytotoxic agents results in a pressing need for accurate response prediction. Although recent studies have identified gene expression signatures associated with response to cytotoxic chemotherapies (Folgueria et al., 2005, Clin. Cancer Res. 11:7434-7443; Ayers et al., 2004, J. Clin. Oncol. 22:2284-2293; Chang et al., 2003, Lancet 362:362-369; Rouzier et al., 2005, Proc. Natl. Acad. Sci. USA 102:8315-8320), the results of these studies remain unvalidated and have not yet had a major effect on clinical practice. In addition to technical issues, such as lack of a standard technology platform and difficulties surrounding the collection of clinical samples, the myriad of cellular processes affected by cytotoxic chemotherapies may hinder the identification of practical and robust gene expression predictors of response to these agents. One exception may be the recent finding by microarray that low mRNA expression of the microtubule-associate protein Tau is predictive of improved response to paclitaxel (Rouzier et al., (2005) supra).

To improve on the limitations of cytotoxic chemotherapies, current approaches to drug design in oncology are aimed at modulating specific cell signaling pathways important for tumor growth and survival (Hahn and Weinberg, 2002, Nat. Rev. Cancer 2:331-341; Hanahan and Weinberg, 2000, Cell 100:57-70; Trosko et al., 2004, Ann. N.Y. Acad. Sci. 1028:192-201).

Although current prognostic criteria and molecular markers provide some guidance in predicting patient outcome and selecting an appropriate course of treatment, a significant need exists for a specific and sensitive method for evaluating cancer prognosis and diagnosis, particularly in early stages. Such a method should specifically distinguish cancer patients with a poor prognosis from those with a good prognosis and permit the identification of high-risk cancer patients who are likely to need aggressive adjuvant therapy.

There is also a need for identifying new parameters that can better predict a patient's sensitivity to treatment or therapy. The classification of patient tumor samples is an important aspect of cancer diagnosis and treatment. The association of a patient's response to drug treatment with molecular and genetic markers can open up new opportunities for drug development in non-responding patients, or distinguish a drug's indication among other treatment choices because of higher confidence in the expected efficacy of the drug. Further, the pre-selection of patients who are likely to respond well to a medicine, drug, or combination therapy may reduce the number of patients needed in a clinical study and/or accelerate the time needed to complete a clinical development program (M. Cockett et al., 2000, Current Opinion in Biotechnology 11:602-609).

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the invention provides a method for classifying a human subject afflicted with a cancer type which is at risk of undergoing an epithelial cell-like to mesenchymal cell-like transition, as having a good prognosis or a poor prognosis, wherein said good prognosis indicates that said subject is expected to have no distant metastases or no reoccurrence within five years of initial diagnosis of said cancer, and wherein said poor prognosis indicates that said subject is expected to have distant metastases or a reoccurrence of cancer within five years of initial diagnosis of said cancer, the method comprising: (a) classifying cancer cells obtained from said human subject as having mesenchymal cell-like qualities or epithelial cell-like qualities on the basis of the expression level of at least 5 of the genes for which markers are listed in any of TABLE 2A, TABLE 2B, TABLE 4A, TABLE 4B and/or at least one of the microRNAs listed in TABLE 9A and TABLE 9B; (b) classifying the human subject as having a good prognosis if the cancer cells are classified according to step (a) as having epithelial cell-like properties, or classifying the human subject as having a poor prognosis if the cancer cells are classified according to step (a) as having mesenchymal cell-like properties; and (c) displaying or outputting to a user, user interface device, computer readable storage medium, or local or remote computer system the classification produced by said classifying step (b).

In another aspect, the invention provides kits comprising PCR primers and/or probes for measuring the gene expression of gene markers useful for classifying cancer cells obtained from said human subject as having mesenchymal cell-like qualities or epithelial cell-like qualities on the basis of the expression level of at least 5 of the genes for which markers are listed in any of TABLE 2A, TABLE 2B, TABLE 4A, TABLE 4B and/or at least one of the microRNAs listed in TABLE 9A and TABLE 9B.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1C show gene expression characteristics of the 93 lung cancer cell lines used to derive the EMT Signature genes. FIG. 1A shows a plot of the 93 lung cancer cell lines distributed by CDH1 gene expression level (y-axis) versus VIM gene expression level (x-axis). FIG. 1B shows a plot of the 93 lung cancer cell lines distributed by differential CDH1 gene expression (y-axis) versus EMT Signature Score (x-axis). FIG. 1C shows a plot of the 93 lung cancer cell lines distributed by EMT Signature Score (y-axis) versus VIM gene expression (x-axis), as described in Example 1;

FIG. 2 shows a waterfall plot of an EMT Signature score for 93 lung tumor cell lines classified as being resistant or sensitive to growth inhibition by exposure to a combination of Tarceva and MK-0646, as described in Example 2;

FIG. 3 shows the intrinsic molecular stratification of gene expression data obtained from 326 human colorectal cancer samples, from the Moffitt Cancer Center, obtained using PC1 classification values. Unsupervised analysis and hierarchical clustering of global gene expression data derived from 326 human colorectal cancer cases identified two major “intrinsic” subclasses of colorectal tumor samples (labeled “epithelial” and “mesenchymal” shown in cyan (lighter greyscale) and magenta (darker greyscale, respectively) distinguished by the first principal component (PC1) representing the most variably expressed genes within the 326 colorectal cancer patients. The subpanel on the far right of the figure shows that the PC1 classification for each colorectal cancer sample is tightly correlated with the EMT Signature Score, as described in Example 3;

FIG. 4 shows the molecular stratification obtained using PC1 classification values as applied to a second independent gene expression data set obtained from 269 colorectal cancer samples (ExPO data set). The subpanel on the far right of the figure shows that the PC1 classification for each colorectal cancer sample is tightly correlated with the EMT Signature Score calculated for each sample, as described in Example 3;

FIG. 5 shows a hierarchical cluster analysis of 100 genes assessed from a text mining approach, as well as several gene signatures (listed in TABLE 5), on gene expression profiles obtained from 326 Moffitt colorectal cancer tumor samples sorted by PC1 score, as described in Example 5;

FIG. 6 shows a scatter plot comparing the values of EMT signature scores (x-axis) versus the values of PC1 (the first principle component) (y-axis) for each tumor sample in the dataset of 326 Moffitt colorectal cancer tumors, as described in Example 5;

FIG. 7A, is a covariance matrix showing that the PC1 signature score correlates well with the EMT Signature score (statistically significant with p value<0.01), disease recurrence, disease progression, and differentiation status, as described in Example 6;

FIG. 7B, shows a Kaplan-Meier Curve of disease-free survival time of colon cancer patients (stages 1, 2, 3 and 4) obtained by performing survival analysis in terms of eventless probability (y-axis), plotted against time measured in months (x-axis) on the cancer patients from which the 326 colorectal tumors from the Moffitt dataset were derived, with the tumor samples stratified into two groups based on whether the PC1 score was below or above the mean, showing that a low PC1 score correlates with a good colon cancer prognosis, and a high PC1 score correlates with a poor colon cancer prognosis, as described in Example 6;

FIG. 8 shows a waterfall plot of cancer recurrence prediction using the PC1 Signature score for patients who contributed samples used to generate the Moffitt Cancer Center colorectal cancer gene expression dataset, as described in Example 6;

FIGS. 9A-9B show a waterfall plot of cancer recurrence prediction using the PC1 Signature score for patients who contributed samples used to generate the Moffitt Cancer Center (MCC) colorectal cancer gene expression dataset. FIG. 9A shows patients' samples classified as Stage 2 colorectal cancer. FIG. 9B shows patients' samples classified as Stage 3 colorectal cancer. Cancer recurrence and non-recurrent patients are defined as described for FIG. 8, as described in Example 6;

FIG. 10A, shows a Kaplan-Meier Curve of metastasis-free survival time of colon cancer patients (stages 2 and 3) showing metastasis-free survival time (recurrence-free time) (y-axis) plotted against time (measured in years) in a dataset obtained from NM (unpublished), wherein the PC1 Score was computed as the difference in mean intensities for the genes that were most positively and negatively correlated to PC1 in the Moffitt colorectal dataset of 326 tumors. The samples were stratified into two groups: “high PC1 Score” or “low PC1 score” depending on whether their PC1 score was above or below the mean PC1 Score on the given dataset, as described in Example 6;

FIG. 10B shows a waterfall plot of PC1 Signature Score and colon cancer recurrence or non-recurrence in a dataset obtained from Lin et al. (2007, Clin. Cancer Res. 13:498-507), as described in Example 6;

FIGS. 11A-11C show a heat map representation of gene expression profile data from Colon, Lung and Pancreas tumor samples. FIG. 11A shows analysis of 104 genes/gene signatures (listed in TABLE 6) on gene expression data from more than 800 primary colorectal cancer tumors sorted by PC1 Signature score. Genes positively correlated with the PC1 Signature score are shown in Red/darker greyscale (Mesenchymal). Genes negatively correlated with the PC1 Signature score are shown in Blue/lighter greyscale (Epithelial). FIG. 11B shows analysis of 82 genes/gene signatures (listed in TABLE 7) on gene expression data from more than 900 primary lung cancer tumors sorted by EMT Signature score. Genes positively correlated with the EMT Signature score are shown in Red/darker greyscale (Mesenchymal). Genes negatively correlated with the EMT Signature score are shown in Blue/lighter greyscale (Epithelial). FIG. 11C shows analysis of 92 genes/gene signatures (listed in TABLE 8) on gene expression data from primary pancreatic tumors sorted by EMT Signature score. Genes positively correlated with the EMT Signature score are shown in Red/darker greyscale (Mesenchymal). Genes negatively correlated with the EMT Signature score are shown in Blue/lighter greyscale (Epithelial), as described in Example 6;

FIG. 12A, shows a summary of the pancreas, lung and colon gene expression profiling datasets presented in FIGS. 11A-C, sorted by cancer type and EMT signature scores. The x-axis shows the number of primary tumor samples grouped by the cancer type (pancreas, lung, colon) and sorted within each cancer type by the EMT signature score, as described in Example 6;

FIG. 12B shows a boxplot analysis of the differential EMT signature scores for colon<lung<pancreas following normalization across all patient samples, as described in Example 6;

FIGS. 13A-13C show covariance matrices showing the relationship of PC1 and EMT Signature scores to the same endpoints as shown in FIG. 7A. FIG. 13A, shows a covariance matrix using a German colorectal cancer dataset from Lin et al. (2007, Clin. Cancer Res. 13:498-507). FIG. 13B shows a covariance matrix using a colon cancer dataset from EXPO. FIG. 13C shows a covariance matrix using a colon cancer dataset from the Netherlands Cancer Institute (NM), as described in Example 6;

FIG. 14A shows a plot of miR-200a expression levels compared to the EMT Signature score from 49 colorectal cancer samples. FIG. 14B shows a waterfall plot of miR-200a levels measured in colorectal tumor samples classified as mesenchymal-like and epithelial-like, as described in Example 7; and

FIG. 15A shows a plot of miR-200b expression levels compared to the EMT Signature scores from 49 colorectal cancer samples. FIG. 15B shows a waterfall plot of miR-200b levels measured in colorectal tumor samples classified as mesenchymal-like and epithelial-like, as described in Example 7.

### DETAILED DESCRIPTION

This section presents a detailed description of the many different aspects and embodiments that are representative of the inventions disclosed herein. This description is by way of several exemplary illustrations, of varying detail and specificity. Other features and advantages of these embodiments are apparent from the additional descriptions provided herein, including the different examples. The provided examples illustrate different components and methodology useful in practicing various embodiments of the invention. The examples are not intended to limit the claimed invention. Based on the present disclosure, the ordinary skilled artisan can identify and employ other components and methodologies useful for practicing the present invention.

### Introduction

Various embodiments of the invention relate to classifying cancer cells as having mesenchymal cell-like qualities or epithelial cell-like qualities (i.e., the EMT status of the cancer cells) on the basis of the expression level of various gene sets, including EMT signature genes, PC1 signature genes, and/or signature microRNAs, for which markers are listed in TABLES 2A, 2A, 4A, 4B, and 9A, 9B, respectively, whose expression patterns correlate with an important characteristic of cancer cells, i.e., whether the cancer cells have gene expression characteristics correlated with “normal” epithelial cells or “normal” mesenchymal cells. Each of the EMT Signature markers or PC1 Signature markers correspond to a gene in the human genome, i.e., each such marker is identifiable as all or a portion of a gene.

In some embodiments of the invention, the sets of markers for detecting EMT Signature genes and/or PC1 Signature genes may be split into two opposing “arms”—the “Mesenchymal” arm (EMT Signature: TABLE 2A; PC1 Signature: TABLE 4A), which are genes that are more highly expressed in mesenchymal cells as compared to epithelial cells, and the “Epithelial” arm (EMT Signature: TABLE 2B; PC1 Signature: TABLE 4B), which are genes that are more highly expressed in epithelial cells as compared to mesenchymal cells. In some embodiments of the invention, the expression levels of the Mesenchymal arm genes (TABLE 2A) and/or the Epithelial arm genes (TABLE 2B) are used to calculate an Epithelial to Mesenchymal Transition (EMT) signature score for a cancer cell, or plurality of cancer cells. In other embodiments of the invention, the expression levels of the Mesenchymal arm (TABLE 4A) and/or the Epithelial arm genes (TABLE 4B) are used to calculate a PC1 (first principal component) signature score for a cancer cell, or plurality of cancer cells.

In some embodiments of the invention, the calculated EMT or PC1 signature scores for cancer cells obtained from a cancer patient are used to predict the likelihood that the cancer patient will respond or be resistant to certain therapeutic treatments. In one embodiment of the invention, patients whose cancer cells are classified as having a low EMT signature score, or a low PC1 signature score, (i.e., have epithelial cell-like properties), are candidates for treatment with inhibitors of Epidermal Growth Factor Receptor signaling pathway (e.g., with exemplary inhibitors described in U.S. Pat. No. 5,747,498; U.S. Reissue Pat. No. RE 41,065) in combination with inhibitors of Insulin-like Growth Factor Receptor signaling pathway (e.g., with exemplary inhibitors Zha and Lackner, 2010, Clin. Cancer Res. 16:2512-17; U.S. Pat. No. 7,241,444; U.S. Pat. No. 7,553,485).

In some embodiments of the invention, the calculated EMT or PC1 signature scores are used to classify a human subject afflicted with a cancer type which is at risk of undergoing an epithelial cell-like to mesenchymal cell-like transition, as having a good prognosis or a poor prognosis. In some embodiments of the invention, patients whose cancer cells are classified as having a low EMT signature score, or a low PC1 signature score (i.e., have epithelial cell-like properties), are classified as having a good prognosis. In some embodiments of the invention, patients whose cancer cells are classified as having a high EMT signature score, or a high PC1 signature score (i.e., have mesenchymal cell-like properties), are classified as having a poor prognosis.

### DEFINITIONS

Unless defined otherwise, all technical and scientific terms used herein have the same meaning as commonly understood to one of ordinary skill in the art to which this invention belongs. The following definitions are provided in order to provide clarity with respect to terms as they are used in the specification and claims to describe various embodiments of the present invention.

As used herein, “oligonucleotide sequences that are complementary to one or more of the genes described herein” refers to oligonucleotides that are capable of hybridizing under stringent conditions to at least part of the nucleotide sequence of said genes. Such hybridizable oligonucleotides will typically exhibit at least about 75% sequence identity at the nucleotide level to said genes, preferably about 80% or 85% sequence identity, or more preferably about 90%, 95%, 96%, 97%, 98% or 99% sequence identity to said genes.

As used herein, the term “bind(s) substantially” refers to complementary hybridization between a nucleic acid probe and a target nucleic acid and embraces minor mismatches that can be accommodated by reducing the stringency of the hybridization media to achieve the desired detection of the target polynucleotide sequence.

As used herein, the term “cancer” means any disease, condition, trait, genotype or phenotype characterized by unregulated cell growth or replication as is known in the art; including leukemias, for example, acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as osteosarcoma, chondrosarcomas, Ewing's sarcoma, fibrosarcomas, giant cell tumors, adamantinomas, and chordomas; brain cancers such as meningiomas, glioblastomas, lower-grade astrocytomas, oligodendrocytomas, pituitary tumors, schwannomas, and Metastatic brain cancers; cancers of the head and neck including various lymphomas such as mantle cell lymphoma, non-Hodgkin's lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, colorectal cancer, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease, and any other cancer or proliferative disease, condition, trait, genotype or phenotype that can respond to the modulation of disease related gene expression in a cell or tissue, alone or in combination with other therapies.

As used herein, “colon cancer,” also called “colorectal cancer” or “bowel cancer,” refers to a malignancy that arises in the large intestine (colon) or the rectum (end of the colon), and includes cancerous growths in the colon, rectum, and appendix, including adenocarcinoma.

As used herein, the phrase “cancer type which is at risk of undergoing an epithelial cell-like to mesenchymal cell-like transition” refers to any cancer type which forms solid tumors from an epithelial cell lineage, such as, for example, lung cancer, colon cancer, pancreatic cancer, breast cancer, ovarian cancer, prostate cancer, esophageal cancer, gastric cancer, small bowel cancer, anal cancer, head and neck cancer, uterine cancer, bladder cancer, kidney cancer, skin cancers (melanoma, squamous cell carcinoma, basal cell carcinoma), sarcomas, and brain cancers.

As used herein, the term “good prognosis” in the context of colon cancer means that a patient is expected to have no distant metastases of a colon tumor within five years of initial diagnosis of colon cancer.

As used herein, the term “poor prognosis” in the context of colon cancer means that a patient is expected to have distant metastases of a colon tumor within five years of initial diagnosis of colon cancer.

As used herein, the term “distant metastasis” means a recurrence of a primary tumor in other organs or tissues than the primary tumor. For example, a distant metastasis for colon cancer includes cancer spreading to a tissue or organ other than colon (e.g., liver, lung).

As used herein, the phrase “hybridizing specifically to” refers to the binding, duplexing or hybridizing of a molecule substantially to or only to a particular nucleotide sequence or sequences under stringent conditions when that sequence is present in a complex mixture (e.g., total cellular) DNA or RNA.

As used herein, the term “marker” means any gene, protein, or an EST derived from that gene, the expression or level of which changes between certain conditions. Where the expression of the gene correlates with a certain condition, the gene is a marker for that condition. Sets of gene expression markers are often referred to as a “signature.”

As used herein, the term “marker-derived polynucleotides” means the RNA transcribed from a marker gene, any cDNA or cRNA produced therefrom, and any nucleic acid derived therefrom, such as a synthetic nucleic acid having a sequence derived from the gene corresponding to the marker gene.

A gene marker is “informative” for a condition, phenotype, genotype or clinical characteristic if the expression of the gene marker is correlated or anti-correlated with the condition, phenotype, genotype or clinical characteristic to a greater degree than would be expected by chance.

As used herein, the term “gene” has its meaning as understood in the art. However, it will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs and microRNAs. For clarity, the term “gene” generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid. In some cases, the gene includes regulatory sequences involved in transcription, or message production or composition. In other embodiments, the gene comprises transcribed sequences that encode for a protein, polypeptide, or peptide. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. In particular embodiments, the transcribed nucleotide sequence comprises at least one functional protein, polypeptide and/or peptide encoding unit. As will be understood by those in the art, this functional term “gene” includes both genomic sequences, RNA or cDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express, using nucleic acid manipulation technology, proteins, polypeptides, domains, peptides, fusion proteins, mutants and/or such like. The sequences which are located 5′ of the coding region and which are present on the mRNA are referred to as 5′ untranslated sequences (“5′UTR”). The sequences which are located 3′ or downstream of the coding region and which are present on the mRNA are referred to as 3′ untranslated sequences, or (“3′UTR”).

As used herein, the term “signature” refers to a set of one or more differentially expressed genes that are statistically significant and characteristic of the biological differences between two or more cell samples, e.g., normal and diseased cells, cell samples from different cell types or tissue, or cells exposed to an agent or not. A signature may be expressed as a number of individual unique probes complementary to signature genes whose expression is detected when a cRNA product is used in microarray analysis or in a PCR reaction. A signature may be exemplified by a particular set of markers.

As used herein, a “similarity value” is a number that represents the degree of similarity between two things being compared. For example, a similarity value may be a number that indicates the overall similarity between a cell sample expression profile using specific phenotype-related biomarkers and a control specific to that template (for instance, the similarity to a “deregulated growth factor signaling pathway” template, where the phenotype is a deregulated growth factor signaling pathway status). The similarity value may be expressed as a similarity metric, such as a correlation coefficient, or may simply be expressed as the expression level difference, or the aggregate of the expression level differences, between a cell sample expression profile and a baseline template.

As used herein, the terms “measuring expression levels,” “obtaining expression level,” and “detecting an expression level” and the like, includes method that quantify a gene expression level of, for example, a transcript of a gene, or a protein encoded by a gene, as well as methods that determine whether a gene of interest is expressed at all. Thus, an assay which provides a “yes” or “no” result without necessarily providing quantification of an amount of expression is an assay that “measures expression” as that term is used herein. Alternatively, a measured or obtained expression level may be expressed as any quantitative value, for example, a fold-change in expression, up or down, relative to a control gene or relative to the same gene in another sample, or a log ratio of expression, or any visual representation thereof, such as, for example, a “heatmap” where a color intensity is representative of the amount of gene expression detected. Exemplary methods for detecting the level of expression of a gene include, but are not limited to, Northern blotting, dot or slot blots, reporter gene matrix (see for example, U.S. Pat. No. 5,569,588) nuclease protection, RT-PCR, microarray profiling, differential display, 2D gel electrophoresis, SELDI-TOF, ICAT, enzyme assay, antibody assay, and the like.

As used herein, a “patient” can mean either a human or non-human animal, preferably a mammal.

As used herein, “subject” refers to an organism, such as a mammal, or to a cell sample, tissue sample or organ sample derived therefrom, including, for example, cultured cell lines, a biopsy, a blood sample, or a fluid sample containing a cell or a plurality of cells. In many instances, the subject or sample derived therefrom comprises a plurality of cell types. In one embodiment, the sample includes, for example, a mixture of tumor and normal cells. In one embodiment, the sample comprises at least 10%, 15%, 20%, et seq., 90%, or 95% tumor cells. The organism may be an animal, including, but not limited to, an animal, such as a cow, a pig, a mouse, a rat, a chicken, a cat, a dog, etc., and is usually a mammal, such as a human.

As used herein, the term “pathway” is intended to mean a set of system components involved in two or more sequential molecular interactions that result in the production of a product or activity. A pathway can produce a variety of products or activities that can include, for example, intermolecular interactions, changes in expression of a nucleic acid or polypeptide, the formation or dissociation of a complex between two or more molecules, accumulation or destruction of a metabolic product, activation or deactivation of an enzyme or binding activity. Thus, the term “pathway” includes a variety of pathway types, such as, for example, a biochemical pathway, a gene expression pathway, and a regulatory pathway. Similarly, a pathway can include a combination of these exemplary pathway types.

As used herein, the term “treating” in its various grammatical forms in relation to the present invention refers to preventing (i.e., chemoprevention), curing, reversing, attenuating, alleviating, minimizing, suppressing, or halting the deleterious effects of a disease state, disease progression, disease causative agent (e.g., bacteria or viruses), or other abnormal condition. For example, treatment may involve alleviating a symptom (i.e., not necessarily all the symptoms) of a disease or attenuating the progression of a disease.

“Treatment of cancer,” as used herein, refers to partially or totally inhibiting, delaying, or preventing the progression of cancer including cancer metastasis; inhibiting, delaying, or preventing the recurrence of cancer including cancer metastasis; or preventing the onset or development of cancer (chemoprevention) in a mammal, for example, a human. The methods of the present invention may be practiced for the treatment of human patients with cancer. However, it is also likely that the methods would be effective in the treatment of cancer in other mammals.

As used herein, the term “therapeutically effective amount” is intended to quantify the amount of the treatment in a therapeutic regiment necessary to treat cancer. This includes combination therapy involving the use of multiple therapeutic agents, such as a combined amount of a first and second treatment where the combined amount will achieve the desired biological response. The desired biological response is partial or total inhibition, delay, or prevention of the progression of cancer including cancer metastasis; inhibition, delay, or prevention of the recurrence of cancer including cancer metastasis; or the prevention of the onset of development of cancer (chemoprevention) in a mammal, for example, a human.

As used herein, the term “displaying or outputting a classification result, prediction result, or efficacy result” means that the results of a gene expression based sample classification or prediction are communicated to a user using any medium, such as for example, orally, writing, visual display, computer readable medium, computer system, or the like. It will be clear to one skilled in the art that outputting the result is not limited to outputting to a user or a linked external component(s), such as a computer system or computer memory, but may alternatively or additionally be outputting to internal components, such as any computer readable medium. Computer readable media may include, but are not limited to, hard drives, floppy disks, CD-ROMs, DVDs, and DATs. Computer readable media does not include carrier waves or other wave forms for data transmission. It will be clear to one skilled in the art that the various sample classification methods disclosed and claimed herein, can, but need not, be computer-implemented, and that, for example, the displaying or outputting step can be done, for example, by communicating to a person orally or in writing (e.g., in handwriting).

### Markers Useful in Classifying Cells and Predicting Response to Therapeutic Agents

Generally, the invention provides signature marker sets (TABLES 2A, 2B, 4A, 4B, 9A, and 9B) whose expression levels within a cancer sample are correlated or anti-correlated with the EMT status of the sample, and methods of use thereof. Various combinations of the gene markers listed in TABLES 2A, 2B, 4A, 4B and/or microRNAs listed in TABLE 9A, and TABLE 9B can be used to measure corresponding gene transcription levels in tumor samples. Depending upon the measured levels of transcription as compared to appropriate control sample transcription levels, tumor cell samples or human subjects from which such samples are obtained, can be classified or sorted into different categories. For example, one aspect of the invention provides methods for predicting the response of a human subject with cancer to a treatment that induces a therapeutically beneficial response if said cancer is classified as having epithelial cell-like qualities based on the levels of transcription measured in the inventive signature gene sets. Another aspect of the invention provides methods for classifying a patient afflicted with a cancer type which is at risk of undergoing an epithelial cell-like to mesenchymal cell-like transition, as having a good prognosis or a poor prognosis based on the EMT status of a cell sample obtained from the patient. Classification of a cancer sample obtained from the patient as having a good prognosis indicates that the patient is expected to have no distant metastases or no reoccurrence of cancer within five years of initial diagnosis of the cancer. In contrast, classification of a cancer sample from the patient as having a poor prognosis indicates that patient is expected to have distant metastases or a reoccurrence of cancer within five years of initial diagnosis of the cancer.

EMT, PC1, and microRNA Signature Markers

In one aspect, the invention provides a set of 310 EMT Signature markers whose expression is correlated with the epithelial to mesenchymal cell transition (EMT) program. Exemplary markers identified as useful for classifying cell samples according to the EMT Signature are listed in TABLES 2A and 2B. In another aspect, the invention provides a set of 243 PC1 Signature markers whose expression is correlated with the EMT Signature score. Exemplary markers identified as useful for classifying cell samples according to the PC1 Signature are listed in TABLES 4A and 4B. In yet another aspect, the invention provides a set of 131 MicroRNA Signature markers whose expression is correlated with the EMT Signature score. Exemplary markers identified as useful for classifying cell samples according to the microRNA Signature are listed in TABLES 9A and 9B.

In some embodiments of the invention, subsets of the EMT Signature markers, PC1 Signature markers, and/or MicroRNA Signature markers may be used. A subset of markers may be selected entirely from one of the inventive signatures (i.e., from the EMT Signature (TABLES 2A and 2B), from the PC1 Signature (TABLES 4A and 4B), or from the microRNA Signature (TABLES 9A and 9B)), or from a combination of two of the three inventive signatures, or from all three of the inventive signatures, (i.e., the EMT Signature, the PC1 Signature, and the microRNA Signature). For example, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, or, 57 or more, 58 or more, 59 or more markers, or 60 or more of the markers listed in one or more of TABLES 2A, 2B, 4A, 4B, 9A and 9B may be used to practice any of the methods disclosed herein. In another embodiment, a subset of microRNAs may be selected from the microRNA Signature (TABLES 9A and 9B). For example, one or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, or 30 or more of the microRNAs listed in TABLES 9A and 9B may be used to practice any of the methods disclosed herein. In some embodiments, the microRNAs included in the miR-200 family are used to practice the methods of the invention.

In some embodiments of the invention, larger subsets of the EMT Signature markers, PC1 Signature markers, and/or microRNA Signature markers may be used. For example, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 80 or more, 85 or more, 90 or more, 95 or more, 100 or more, 125 or more, 150 or more, 175 or more, 200 or more, 225 or more, 250 or more, 275 or more, 300 or more, 350 or more, 400 or more, 450 or more, or 500 or more of the markers listed in one or more of TABLES 2A, 2B, 4A, 4B, 9A, and 9B may be used to practice any of the methods disclosed herein. In another embodiment, all of the EMT Signature markers listed in TABLES 2A and 2B are used to practice any of the methods disclosed herein. In another embodiment, all of the PC1 markers listed in TABLES 4A and 4B are used to practice any of the methods disclosed herein. In yet another embodiment, all of the microRNA Signature markers listed in TABLES 9A and 9B are used to practice any of the methods disclosed herein.

### Prediction of Drug Response

In one aspect, the invention provides a method of predicting the response of a human subject with cancer to a drug treatment that induces a therapeutically beneficial response in cancer cells classified as having epithelial cell-like qualities, said method comprising classifying cancer cells obtained from the human subject as having mesenchymal cell-like qualities or epithelial cell-like qualities, on the basis of the expression levels of at least 5 or more of the genes for which markers are listed in any of TABLE 2A, TABLE 2B, TABLE 4A, TABLE 4B, TABLE 9A and TABLE 9B, wherein said human subject is predicted to respond positively to said treatment if said cell sample is classified as having epithelial cell-like properties.

In one embodiment, the classifying comprises the following two steps. The first classification step (i) involves calculating a measure of similarity between a first expression profile and a mesenchymal cell-like template, the first expression profile comprising the expression levels of a first plurality of genes in an isolated cell sample derived from the human subject, the mesenchymal cell-like template comprising expression levels of the first plurality of genes that are average expression levels of the respective genes in a plurality of human control cell samples that have mesenchymal cell-like qualities, the first plurality of genes consisting of at least 5 of the genes for which markers are listed in one or more of TABLE 2A, TABLE 4A and TABLE 9A. In accordance with this embodiment, the second classification step (ii) involves classifying the cancer cells as having the mesenchymal cell-like properties if the first expression profile has a high similarity to the mesenchymal cell-like template, or classifying the cell sample as having the epithelial cell-like properties if the first expression profile has a low similarity to the mesenchymal cell-like template, wherein the first expression profile has a high similarity to the mesenchymal cell-like template if the similarity to the mesenchymal cell-like template is above a predetermined threshold, or has a low similarity to the mesenchymal cell-like template if the similarity to the mesenchymal cell-like template is below the predetermined threshold. The human subject is predicted to respond to treatment if the cell sample is classified as having epithelial cell-like properties. The methods of this aspect of the invention may be carried out on a suitably programmed computer and optionally the classification result is displayed or outputted to a user, user interface device, a computer readable storage medium, or a local or remote computer system.

In another embodiment of this aspect of the invention, the classifying step comprises (i) calculating a measure of similarity between a first expression profile and an epithelial cell-like template, said first expression profile comprising the expression levels of a first plurality of genes in an isolated cell sample derived from said human subject, said epithelial cell-like template comprising expression levels of said first plurality of genes that are average expression levels of the respective genes in a plurality of human control cell samples that have epithelial cell-like qualities, said first plurality of genes consisting of at least 5 of the genes for which markers are listed in one or more of TABLE 2B, TABLE 4B, and TABLE 9B; and (ii) classifying said cancer cells as having said epithelial cell-like properties if said first expression profile has a high similarity to said epithelial cell-like template, or classifying said cell sample as having said mesenchymal cell-like properties if said first expression profile has a low similarity to said epithelial cell-like template; wherein said first expression profile has a high similarity to said epithelial cell-like template if the similarity to said epithelial cell-like template is above a predetermined threshold, or has a low similarity to said epithelial cell-like template if the similarity to said epithelial cell-like template is below said predetermined threshold.

In another embodiment, the methods according to this aspect of the invention comprise classifying cancer cells obtained from a human subject as having mesenchymal cell-like qualities or epithelial cell-like qualities by calculating an EMT Signature Score for the cancer cells isolated from the human subject by a method comprising: (i) calculating a differential expression value of a first expression level of each of a first plurality of genes and each of a second plurality of genes in the isolated cancer cell sample derived from the human subject relative to a second expression level of each of said first plurality of genes and each of said second plurality of genes in a human control cell sample, said first plurality of genes consisting of at least 5 of the genes for which markers are listed in TABLE 2A (Mesenchymal Arm) and said second plurality of genes consisting of at least 5 of the genes for which markers are listed in TABLE 2B (Epithelial Arm); (ii) calculating the mean differential expression values of the expression levels of said first plurality of genes and said second plurality of genes; and (iii) subtracting said mean differential expression value of said second plurality of genes from said mean differential expression value of said first plurality of genes to obtain said EMT Signature Score. The cancer cell sample is then classified as having mesenchymal cell-like properties if said obtained EMT Signature Score is at or above a first predetermined threshold and is statistically significant; or said cancer cell sample is classified as having epithelial cell-like properties if said obtained EMT Signature Score is at or below a second predetermined threshold and is statistically significant.

In another embodiment, the methods according to this aspect of the invention comprise classifying cancer cells obtained from a human subject as having mesenchymal cell-like qualities or epithelial cell-like qualities by calculating a PC1 Signature Score for the cancer cells isolated from the human subject by a method comprising: (i) calculating a differential expression value of a first expression level of each of a first plurality of genes and each of a second plurality of genes in the isolated cancer cell sample derived from the human subject relative to a second expression level of each of said first plurality of genes and each of said second plurality of genes in a human control cell sample, said first plurality of genes consisting of at least 5 of the genes for which markers are listed in TABLE 4A (Mesenchymal Arm) and said second plurality of genes consisting of at least 5 of the genes for which markers are listed in TABLE 4B (Epithelial Arm); (ii) calculating the mean differential expression values of the expression levels of said first plurality of genes and said second plurality of genes; and (iii) subtracting said mean differential expression value of said second plurality of genes from said mean differential expression value of said first plurality of genes to obtain said PC1 Signature Score. The cancer cell sample is then classified as having mesenchymal cell-like properties if said obtained PC1 Signature Score is at or above a first predetermined threshold and is statistically significant; or said cancer cell sample is classified as having epithelial cell-like properties if said obtained PC1 Signature Score is at or below a second predetermined threshold and is statistically significant.

In one embodiment of the invention, patients whose cancer cells are classified as having a low EMT signature score, or a low PC1 signature score (i.e., as having epithelial cell-like properties), are candidates for treatment with inhibitors of Epidermal Growth Factor Receptor signaling pathway (U.S. Pat. No. 5,747,498; U.S. Reissue Pat. No. RE 41,065) in combination with inhibitors of Insulin-like Growth Factor Receptor signaling pathway (Zha and Lackner, 2010, Clin. Cancer Res. 16:2512-17; U.S. Pat. No. 7,241,444; U.S. Pat. No. 7,553,485).

In one particular embodiment of the invention, the Epidermal Growth Factor Receptor inhibitor is a kinase inhibitor, erlotinib, with the chemical name N-(3-ethynylphenyl)-6,7-bis (2-methoxyethoxy)-4-quinazolinamine (U.S. Pat. No. 5,747,498; U.S. Reissue Pat. No. RE 41,065), the disclosures of which are herein incorporated by reference.

In another particular embodiment of the invention, the Insulin-like Growth Factor Receptor signaling pathway inhibitor is monoclonal antibody MK-0646 (dalotuzumab) (U.S. Pat. No. 7,241,444; U.S. Pat. No. 7,553,485), the disclosures of which are herein incorporated by reference.

The invention provides a set of markers useful for distinguishing samples from those patients who are predicted to respond to treatment with a combination of agents that inhibit the Epidermal Growth Factor Receptor and Insulin-like Growth Factor Receptor from patients who are not predicted to respond to treatment with a combination of agents that inhibit the Epidermal Growth Factor Receptor and Insulin-like Growth Factor Receptor. Thus, the invention further provides a method for using the inventive EMT and PC1 Signature marker sets for determining whether an individual with cancer is predicted to respond to treatment with a combination of agents that inhibit the Epidermal Growth Factor Receptor and Insulin-like Growth Factor Receptor.

In one embodiment, the invention provides for a method of predicting response of a cancer patient to a combination of agents that inhibit the Epidermal Growth Factor Receptor and Insulin-like Growth Factor Receptor comprising: (1) comparing the level of expression of at least 5 or more of the genes for which markers are listed in TABLES 4A, 4B, 9A, and 9B in a sample taken from the individual to the level of expression of the same genes in a standard or control, where the standard or control levels represent those found in a sample having an epithelial cell like phenotype; and (2) determining whether the level of the gene marker-related polynucleotides in the sample from the individual is significantly different than that of the control, wherein if no substantial difference is found, the patient is predicted to respond to treatment with the combination of agents that inhibit the Epidermal Growth Factor Receptor and Insulin-like Growth Factor Receptor, and if a substantial difference is found, the patient is predicted not to respond to treatment with the combination of agents that inhibit the Epidermal Growth Factor Receptor and Insulin-like Growth Factor Receptor. Persons of skill in the art will readily see that the standard or control levels may be from a tumor sample having a mesenchymal cell-like phenotype. In a more specific embodiment, both controls are run. In case the pool is not pure “epithelial cell-like phenotype” or “mesenchymal cell-like phenotype,” a set of experiments involving individuals with known combination agent responder status should be hybridized against the pool to define the expression templates for the predicted responder and predicted non-responder groups. Each individual with unknown outcome is hybridized against the same pool and the resulting expression profile is compared to the templates to predict its outcome.

The inventive methods can use the complete set of genes for which markers are listed in TABLES 2A, 2B, 4A, 4B, 9A, and 9B, however, markers listed in both TABLES 2A and 4A or TABLES 2B and 4B need only be used once. In other embodiments, subsets of the genes for which markers are listed in TABLES 2A, 2B, 4A, 4B, 9A, and 9B may also be used. In another embodiment, a subset of at least 5, 10, 20, 30, 40, 50, 75, or 100 markers drawn from TABLES 2A, 2B, 4A, 4B, 9A, and 9B, can be used to predict the response of a subject to an agent that modulates the growth factor signaling pathway or assign treatment to a subject.

In another embodiment, the above method of determining the EMT status of a cancer sample obtained from a subject to predict treatment response or assign treatment uses two “arms” of the EMT signature, PC1 signature and/or MicroRNA signature markers. The “mesenchymal” arm comprises the genes whose expression goes up with the transition of tissue to mesenchymal like cell characteristics (growth factor pathway activation (see TABLES 2A, 4A, and 9A)), and the “epithelial” arm comprises the genes whose expression goes down with transition of tissue to mesenchymal like cell characteristics (see TABLES 2B, 4B, and 9B). Alternatively, the above method of determining EMT status uses two “arms” of the 310 EMT Signature markers listed in TABLES 2A and 2B, including the “mesenchymal” arm comprising or consisting of 149 markers (see TABLE 2A) and the “epithelial” arm comprising or consisting of 161 markers (see TABLE 2B). In an alternative embodiment, EMT status is determined using two “arms” of the 243 PC1 Signature markers listed in TABLES 4A and 4B, including the “mesenchymal” arm comprising or consisting of 124 markers (see TABLE 4A) and the “epithelial” arm comprising or consisting of 119 markers (see TABLE 4B). In yet another alternative embodiment, EMT status is determined using two “arms” of the 131 MicroRNA markers listed in TABLES 9A and 9B, including the “mesenchymal” arm comprising or consisting of 74 markers (see TABLE 9A) and the “epithelial” arm comprising or consisting of 57 markers (see TABLE 9B).

When comparing an individual sample with a standard or control, the expression value of marker X in the sample is compared to the expression value of marker X in the standard or control. For each gene in a set of inventive markers, log(10) ratio is created for the expression value in the individual sample relative to the standard or control. An EMT signature “score” is calculated by determining the mean log(10) ratio of the genes in the “up” arm of the signature, here referred to as the “mesenchymal” and then subtracting the mean log(10) ratio of the genes in the “down” arm, here referred to as the “epithelial.” If the EMT signature score is above a pre-determined threshold, then the sample is considered to have a mesenchymal-like EMT status. In one embodiment of the invention, the pre-determined threshold is set at 0. The pre-determined threshold may also be the mean, median, or a percentile of EMT signature scores of a collection of samples or a pooled sample used as a standard of control. To determine if the EMT signature score is significant, an ANOVA calculation is performed (for example, a two tailed t-test, Wilcoxon rank-sum test, Kolmogorov-Smirnov test, etc.), in which the expression values of the genes in the two opposing arms (Mesenchymal and Epithelial) are compared to one another. For example, if the two tailed t-test is used to determine whether the mean log(10) ratio of the genes in the “Mesenchymal” arm is significantly different than the mean log(10) ratio of the genes in the “Epithelial” arm, a p-value of <0.05 indicates that the signature in the individual sample is significantly different from the standard or control.

It will be recognized by those skilled in the art that other differential expression values, besides log(10) ratio, may be used for calculating a signature score, as long as the value represents an objective measurement of transcript abundance of the genes. Examples include, but are not limited to: xdev, error-weighted log(ratio), and mean subtracted log(intensity).

One embodiment of the invention provides a method of predicting a therapeutically beneficial response of a cancer patient to a combination of agents that inhibit the Epidermal Growth Factor Receptor and Insulin-like Growth Factor Receptor if said cancer is classified as having epithelial cell-like qualities, said method comprising: (a) calculating an EMT Signature Score by a method comprising: i) calculating a differential expression value of a first expression level of each of a first plurality of genes and each of a second plurality of genes in an isolated cancer cell sample derived from the human subject prior to treatment with the combination of agents relative to a second expression level of each of the first plurality of genes and each of the second plurality of genes in a human control cell sample, the first plurality of genes consisting of at least 5 or more of the genes for which markers are listed in TABLES 2A, 4A, and 9A (Mesenchymal Arm) and the second plurality of genes consisting of at least 5 or more of the genes for which markers are listed in TABLES 2B, 4B, and 9A (Epithelial Arm); ii) calculating the mean differential expression values of the expression levels of the first plurality of genes and the second plurality of genes; and iii) subtracting the mean differential expression value of the second plurality of genes from the mean differential expression value of the first plurality of genes to obtain the EMT Signature Score; (b) classifying the cancer cell sample as having mesenchymal cell-like properties if the obtained EMT Signature Score is at or above a first predetermined threshold and is statistically significant; or classifying said cancer cell sample as having epithelial cell-like properties if the obtained EMT Signature Score is at or below a second predetermined threshold and is statistically significant; wherein the human subject is predicted to respond to the treatment if the cell sample is classified as having epithelial cell-like properties. Optionally, the EMT Signature Score and/or EMT classification status, i.e., mesenchymal cell-like properties or epithelial cell-like properties, is displayed; or output to a user, a user interface device, a computer readable storage medium, or a local or remote computer system.

In one embodiment, the first plurality of genes consists of at least 6, 7, 8, 9, or 10 or more of the genes for which markers are listed in TABLES 2A, 4A, and 9A. In another embodiment, the second plurality of genes consists of at least 6, 7, 8, 9, or 10 or more of the genes for which markers are listed in TABLES 2B, 4B, and 9B.

In an alternative embodiment, the first plurality of genes consists of at least 11, 12, 13, 14, 15, 16, 17, 18, 19, or 20 or more of the genes for which markers are listed in TABLES 2A, 4A, and 9A. In an alternative embodiment, the second plurality of genes consists of at least 11, 12, 13, 14, 15, 16, 17, 18, 19, or 20 or more of the genes for which markers are listed in TABLES 2B, 4B, and 9B.

In an yet another embodiment, the first plurality of genes consists of at least 21, 22, 23, 24, 25, 26, 27, 28, 29, or 30 or more of the genes for which markers are listed in TABLES 2A, 4A, and 9A. In an alternative embodiment, the second plurality of genes consists of at least 21, 22, 23, 24, 25, 26, 27, 28, 29, or 30 or more of the genes for which markers are listed in TABLES 2B, 4B, and 9B.

In another embodiment, the first plurality of genes consists of all of the genes for which markers are listed in TABLES 2A, 4A, and 9A. In another embodiment, the second plurality of genes consists of all of the genes for which markers are listed in TABLES 2B, 4B, and 9B. In another embodiment, the first plurality of genes consists of all of the genes for which markers are listed in TABLE 2A and the second plurality of genes consists of all of the genes for which markers are listed in TABLE 2B.

In one embodiment of the invention, the differential expression value is expressed as a log(10) ratio. In another embodiment of the invention, the first and second predetermined threshold is 0. Alternatively, the first predetermined threshold is set from 0.1 to 0.3. In another embodiment, the second predetermined threshold is set from ⁻0.1 to ⁻0.3. In one embodiment, the EMT Signature Score is statistically significant if it has a p-value of less than 0.05.

In methods where similarity between a gene expression profile obtained from a cancer sample and the mesenchymal cell-like template or the epithelial cell-like template are used to perform the EMT classification step, the degree of similarity can be determined using any method known in the art. For example, Dai et al. describes a number of different ways of calculating gene expression templates from signature marker sets useful in classifying breast cancer patients (U.S. Pat. No. 7,171,311; WO2002103320; WO2005086891; WO2006015312; WO2006084272). Similarly, Linsley et al. (US 20030104426) and Radish et al. (US 20070154931) disclose signature marker sets and methods of calculating gene expression templates useful in classifying chronic myelogenous leukemia patients.

For example, in one embodiment, the similarity is represented by a correlation coefficient between the sample profile and the template. In one embodiment, a correlation coefficient above a correlation threshold indicates high similarity, whereas a correlation coefficient below the threshold indicates low similarity. In some embodiments, the correlation threshold is set as 0.3, 0.4, 0.5, or 0.6. In another embodiment, similarity between a sample profile and a template is represented by a distance between the sample profile and the template. In one embodiment, a distance below a given value indicates high similarity, whereas a distance equal to or greater than the given value indicates low similarity.

In some embodiments of the invention methods described herein, subsets of the EMT Signature markers (TABLES 2A and 2B), PC1 Signature markers (TABLES 4A and 4B), and/or MicroRNA Signature markers (TABLES 9A and 9B) may be used. The subset of markers may be selected entirely from one of the inventive signatures, i.e., from the EMT Signature, or from a combination of all three of the inventive signatures, i.e., the EMT Signature, the PC1 Signature, and the MicroRNA Signature. For example, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, or, 57 or more, 58 or more, 59 or more markers, 60 or more of the markers listed in TABLES 2A, 2B, 4A, 4B, 9A, and 9B may be used to practice any of the methods disclosed herein. In other embodiments of the invention, larger gene subsets of the EMT Signature markers, PC1 Signature markers, and/or MicroRNA Signature markers may be used. For example, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 80 or more, 85 or more, 90 or more, 95 or more, 100 or more, 125 or more, 150 or more, 175 or more, 200 or more, 225 or more, 250 or more, 275 or more, 300 or more, 350 or more, 400 or more, 450 or more, 500 or more of the markers listed in TABLES 2A, 2B, 4A, 4B, 9A, and 9B may be used to practice any of the methods disclosed herein. In another embodiment, all of the markers listed in TABLES 2A and 2B are used to practice any of the methods disclosed herein. In another embodiment, all of the markers listed in TABLES 4A and 4B are used to practice any of the methods disclosed herein. In yet another embodiment, all of the markers listed in TABLES 9A and 9B are used to practice any of the methods disclosed herein.

Determination of EMT, PC1, and miRNA Signature Marker Expression Levels

The expression levels of the gene markers in a sample may be determined by any means known in the art. The expression level may be determined by isolating and determining the level (i.e., amount) of nucleic acid corresponding to each gene marker. Alternatively, or additionally, the level of specific proteins encoded by a nucleic acid corresponding to each gene marker may be determined.

The level of expression of specific marker genes can be accomplished by determining the amount of mRNA, or polynucleotides derived therefrom, present in a sample. Any method for determining RNA levels can be used. For example, RNA is isolated from a sample and separated on an agarose gel. The separated RNA is then transferred to a solid support, such as a filter. Nucleic acid probes representing one or more markers are then hybridized to the filter by northern hybridization, and the amount of marker-derived RNA is determined. Such determination can be visual, or machine-aided, for example, by use of a densitometer. Another method of determining RNA levels is by use of a dot-blot or a slot-blot. In this method, RNA from a sample, or nucleic acid derived therefrom, is labeled. The RNA or nucleic acid derived therefrom is then hybridized to a filter containing oligonucleotides derived from one or more marker genes, wherein the oligonucleotides are placed upon the filter at discrete, easily-identifiable locations. Hybridization, or lack thereof, of the labeled RNA to the filter-bound oligonucleotides is determined visually or by densitometer. Polynucleotides can be labeled using a radiolabel or a fluorescent (i.e., visible) label.

For example, reverse transcription followed by PCR (referred to as RT-PCR) can be used to measure gene expression. RT-PCR involves the PCR amplification of a reverse transcription product, and can be used, for example, to amplify very small amounts of any kind of RNA (e.g., mRNA, rRNA, tRNA). RT-PCR is described, for example, in Chapters 6 and 8 of The Polymerase Chain Reaction, Mullis, K. B., et al., Eds., Birkhauser, 1994, the cited chapters of which publication are incorporated herein by reference.

Again by way of example, ArrayPlate™ kits (sold by High Throughput Genomics, Inc., 6296 E. Grant Road, Tucson, Ariz. 85712) can be used to measure gene expression. In brief, the ArrayPlate™ mRNA assay combines a nuclease protection assay with array detection. Cells in microplate wells are subjected to a nuclease protection assay. Cells are lysed in the presence of probes that bind targeted mRNA species. Upon addition of Si nuclease, excess probes and unhybridized mRNA are degraded, so that only mRNA:probe duplexes remain. Alkaline hydrolysis destroys the mRNA component of the duplexes, leaving probes intact. After the addition of a neutralization solution, the contents of the processed cell culture plate are transferred to another ArrayPlate™ called a programmed ArrayPlate™. ArrayPlates™ contain a 16-element array at the bottom of each well. Each array element comprises a position-specific anchor oligonucleotide that remains the same from one assay to the next. The binding specificity of each of the 16 anchors is modified with an oligonucleotide, called a programming linker oligonucleotide, which is complementary at one end to an anchor and at the other end to a nuclease protection probe. During a hybridization reaction, probes transferred from the culture plate are captured by immobilized programming linker. Captured probes are labeled by hybridization with a detection linker oligonucleotide, which is in turn labeled with a detection conjugate that incorporates peroxidase. The enzyme is supplied with a chemiluminescent substrate, and the enzyme-produced light is captured in a digital image. Light intensity at an array element is a measure of the amount of corresponding target mRNA present in the original cells. The ArrayPlate™ technology is described in Martel, R. R., et al., Assay and Drug Development Technologies 1(1):61-71, 2002, which publication is incorporated herein by reference.

By way of further example, DNA microarrays can be used to measure gene expression. In brief, a DNA microarray, also referred to as a DNA chip, is a microscopic array of DNA fragments, such as synthetic oligonucleotides, disposed in a defined pattern on a solid support, wherein they are amenable to analysis by standard hybridization methods (see Schena, BioEssays 18:427, 1996). Exemplary microarrays and methods for their manufacture and use are set forth in T. R. Hughes et al., Nature Biotechnology 19:342-347, April 2001, which publication is incorporated herein by reference.

Finally, expression of marker genes in a number of tissue specimens may be characterized using a “tissue array” (Kononen et al., 1998, Nat. Med 4:844-847). In a tissue array, multiple tissue samples are assessed on the same microarray. The arrays allow in situ detection of RNA and protein levels; consecutive sections allow the analysis of multiple samples simultaneously.

These examples are not intended to be limiting; other methods of determining RNA abundance are known in the art.

To determine the (increased or decreased) expression levels of genes in the practice of the present invention, any method known in the art may be utilized. In one embodiment of the invention, expression based on detection of RNA which hybridizes to the genes identified and disclosed herein is used. This is readily performed by any RNA detection or amplification method known or recognized as equivalent in the art such as, but not limited to, reverse transcription-PCR, the methods disclosed in U.S. patent application Ser. No. 10/062,857 (filed on Oct. 25, 2001) as well as U.S. Provisional Patent Application Nos. 60/298,847 (filed Jun. 15, 2001) and 60/257,801 (filed Dec. 22, 2000), and methods to detect the presence, or absence, of RNA stabilizing or destabilizing sequences.

Alternatively, expression based on detection of DNA status may be used. Detection of the DNA of an identified gene as may be used for genes that have increased expression in correlation with a particular outcome. This may be readily performed by PCR based methods known in the art, including, but not limited to, Q-PCR. Conversely, detection of the DNA of an identified gene as amplified may be used for genes that have increased expression in correlation with a particular treatment outcome. This may be readily performed by PCR based, fluorescent in situ hybridization (FISH) and chromosome in situ hybridization (CISH) methods known in the art.

### Real-Time PCR

In practice, a gene expression-based expression assay based on a small number of genes (i.e., about 1 to 3000 genes) can be performed with relatively little effort using existing quantitative real-time PCR technology familiar to clinical laboratories. Quantitative real-time PCR measures PCR product accumulation through a dual-labeled fluorogenic probe. A variety of normalization methods may be used, such as an internal competitor for each target sequence, a normalization gene contained within the sample, or a housekeeping gene. Sufficient RNA for real time PCR can be isolated from low milligram quantities from a subject. Quantitative thermal cyclers may now be used with microfluidics cards preloaded with reagents making routine clinical use of multigene expression-based assays a realistic goal.

The gene markers of the EMT, PC1 and EMT miRNA signatures or subset of genes selected from these signatures, which are assayed according to the present invention, are typically in the form of total RNA or mRNA or reverse transcribed total RNA or mRNA. General methods for total and mRNA extraction are well known in the art and are disclosed in standard textbooks of molecular biology, including Ausubel et al., Current Protocols of Molecular Biology, John Wiley and Sons (1997). RNA isolation can also be performed using purification kit, buffer set, and protease from commercial manufacturers, such as Qiagen (Valencia, Calif.) and Ambion (Austin, Tex.), according to the manufacturer's instructions.

TAQman quantitative real-time PCR can be performed using commercially available PCR reagents (Applied Biosystems, Foster City, Calif.) and equipment, such as ABI Prism 7900HT Sequence Detection System (Applied Biosystems) according the manufacturer's instructions. The system consists of a thermocycler, laser, charge-coupled device (CCD), camera, and computer. The system amplifies samples in a 96-well or 384-well format on a thermocycler. During amplification, laser-induced fluorescent signal is collected in real-time through fiber-optics cables for all 96 wells, and detected at the CCD. The system includes software for running the instrument and for analyzing the data.

Based upon the marker gene sets provided in various embodiments of the present invention, a real-time PCR TAQman assay can be used to make gene expression measurements and perform the classification and sorting methods described herein. As is apparent to a person of skill in the art, a wide variety of oligonucleotide primers and probes that are complementary to or hybridize to the signature markers listed in TABLE 2A, TABLE 2B, TABLE 4A, TABLE 4B, TABLE 9A, and TABLE 9B, may be selected based upon the biomarker transcript sequences set forth in the Sequence Listing.

In some embodiments, expression level of the microRNAs or subset of microRNAs for which markers are set forth in TABLES 9A and 9B using the methods disclosed in U.S. Patent Application Publication No. 2007/0292878 and U.S. Patent Application Publication No. 2009/0123912, each of which is herein incorporated by reference.

### Microarrays

In some embodiments, polynucleotide microarrays are used to measure expression so that the expression status of each of the markers in one or more of the inventive gene sets, described herein, is assessed simultaneously. The microarrays of the invention preferably comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more of the EMT and/or PC1 Signature markers, and/or miRNA Signature Markers or all of the EMT and/or PC1 markers, and/or miRNA Signature Markers or any combination or subcombination of EMT and/or PC1 and/or miRNA Signature markers. The actual number of informative markers the microarray comprises will vary depending upon the particular condition of interest, and, optionally, the number of EMT and/or PC1 and/or miRNA Signature markers found to result in the least Type I error, Type II error, or Type I and Type II error in determination of an endpoint phenotype. As used herein, “Type I error” means a false positive and “Type II error” means a false negative; in the example of prediction of therapeutic response to exposure to an agent, Type I error is the mis-characterization of an individual with a therapeutic response to the agent as having being a non-responder to treatment, and Type II error is the mis-characterization of an individual with no response to treatment with the agent as having a therapeutic response.

Polynucleotides capable of specifically or selectively binding to the mRNA transcripts encoding the markers of the invention are also contemplated. For example: oligonucleotides, cDNA, DNA, RNA, PCR products, synthetic DNA, synthetic RNA, or other combinations of naturally occurring or modified nucleotides which specifically and/or selectively hybridize to one or more of the RNA products of the biomarker of the invention are useful in accordance with the invention.

In a preferred embodiment, the oligonucleotides, cDNA, DNA, RNA, PCR products, synthetic DNA, synthetic RNA, or other combinations of naturally occurring or modified nucleotides or oligonucleotides which both specifically and selectively hybridize to one or more of the RNA products of the marker of the invention are used.

### Microarray Hybridization

In one embodiment of the invention, the polynucleotide used to measure the RNA products of the invention can be used as nucleic acid members stably associated with a support to comprise an array according to one aspect of the invention. The length of a nucleic acid member can range from 8 to 1000 nucleotides in length and are chosen so as to be specific for the RNA products of the EMT and/or PC1 Signature markers of the invention. In one embodiment, these members are selective for the RNA products of the invention. The nucleic acid members may be single or double stranded, and/or may be oligonucleotides or PCR fragments amplified from cDNA. Preferably oligonucleotides are approximately 20-30 nucleotides in length. ESTs are preferably 100 to 600 nucleotides in length. It will be understood by a person skilled in the art that one can utilize portions of the expressed regions of the biomarkers of the invention as a probe on the array. More particularly, oligonucleotides complementary to the genes of the invention and or cDNA or ESTs derived from the genes of the invention are useful. For oligonucleotide based arrays, the selection of oligonucleotides corresponding to the gene of interest which are useful as probes is well understood in the art. More particularly, it is important to choose regions which will permit hybridization to the target nucleic acids. Factors such as the Tm of the oligonucleotide, the percent GC content, the degree of secondary structure and the length of nucleic acid are important factors. See, for example, U.S. Pat. No. 6,551,784.

The measuring of the expression of the RNA product of the invention, can be done by using those polynucleotides which are specific and/or selective for the RNA products of the invention to quantitate the expression of the RNA product. In a specific embodiment of the invention, the polynucleotides which are specific to and/or selective for the RNA products are probes or primers. In one embodiment, these polynucleotides are in the form of nucleic acid probes which can be spotted onto an array to measure RNA from the sample of an individual to be measured. In another embodiment, commercial arrays can be used to measure the expression of the RNA product. In yet another embodiment, the polynucleotides which are specific and/or selective for the RNA products of the invention are used in the form of probes and primers in techniques such as quantitative real-time RT PCR, using for example, SYBR®Green, or using TaqMan® or Molecular Beacon techniques, where the polynucleotides used are used in the form of a forward primer, a reverse primer, a TaqMan labeled probe or a Molecular Beacon labeled probe.

In embodiments where a smaller number of genes (e.g., less than 10 genes) are to be analyzed, the nucleic acid derived from the sample cell(s) may be preferentially amplified by use of appropriate primers such that only the genes to be analyzed are amplified to reduce background signals from other genes expressed in the breast cell. Alternatively, and where multiple genes are to be analyzed or where very few cells (or one cell) are used, the nucleic acid from the sample may be globally amplified before hybridization to the immobilized polynucleotides. Of course RNA, or the cDNA counterpart thereof, may be directly labeled and used, without amplification, by methods known in the art.

### Use of a Microarray

A “microarray” is a linear or two-dimensional array of preferably discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, preferably at least about 50/cm², more preferably at least about 100/cm², even more preferably at least about 500/cm², but preferably below about 1,000/cm². Preferably, the arrays contain less than about 500, about 1000, about 1500, about 2000, about 2500, or about 3000 immobilized polynucleotides in total. As used herein, a DNA microarray is an array of oligonucleotides or polynucleotides placed on a chip or other surfaces used to hybridize to amplified or cloned polynucleotides from a sample. Since the position of each particular group of primers in the array is known, the identities of sample polynucleotides can be determined based on their binding to a particular position in the microarray.

Determining gene expression levels may be accomplished utilizing microarrays. Generally, the following steps may be involved: (a) obtaining an mRNA sample from a subject and preparing labeled nucleic acids therefrom (the “target nucleic acids” or “targets”); (b) contacting the target nucleic acids with an array under conditions sufficient for the target nucleic acids to bind to the corresponding probes on the array, for example, by hybridization or specific binding; (c) optional removal of unbound targets from the array; (d) detecting the bound targets, and (e) analyzing the results, for example, using computer based analysis methods. As used herein, “nucleic acid probes” or “probes” are nucleic acids attached to the array, whereas “target nucleic acids” are nucleic acids that are hybridized to the array.

In yet another embodiment of the invention, all or part of a disclosed EMT and/or PC1 Signature marker sequence may be amplified and detected by methods such aspolymerase chain reaction (PCR) and variations thereof, such as, but not limited to, quantitative PCR (Q-PCR), reverse transcription PCR (RT-PCR), and real-time PCR, optionally real-time RT-PCR. Such methods would utilize one or two primers that are complementary to portions of a disclosed sequence, where the primers are used to prime nucleic acid synthesis.

The newly synthesized nucleic acids are optionally labeled and may be detected directly or by hybridization to a polynucleotide of the invention.

The nucleic acid molecules may be labeled to permit detection of hybridization of the nucleic acid molecules to a microarray. That is, the probe may comprise a member of a signal producing system and thus is detectable, either directly or through combined action with one or more additional members of a signal producing system. For example, the nucleic acids may be labeled with a fluorescently labeled dNTP (see, e.g., Kricka, 1992, Nonisotopic DNA Probe Techniques, Academic Press San Diego, Calif.), biotinylated dNTPs, or rNTP followed by addition of labeled streptavidin, chemiluminescent labels, or isotopes. Another example of labels include “molecular beacons” as described in Tyagi and Kramer (Nature Biotech. 14:303, 1996). The newly synthesized nucleic acids may be contacted with polynucleotides (containing sequences) of the invention under conditions which allow for their hybridization. Hybridization may be also be determined, for example, by plasmon resonance (see, e.g., Thiel, et al. Anal. Chem. 69:4948-4956, 1997).

In one embodiment, a plurality, e.g., 2 sets, of target nucleic acids are labeled and used in one hybridization reaction (“multiplex” analysis). For example, one set of nucleic acids may correspond to RNA from one cell and another set of nucleic acids may correspond to RNA from another cell. The plurality of sets of nucleic acids may be labeled with different labels, for example, different fluorescent labels (e.g., fluorescein and rhodamine) which have distinct emission spectra so that they can be distinguished. The sets may then be mixed and hybridized simultaneously to one microarray (see, e.g., Shena, et al., Science 270:467-470, 1995).

A number of different microarray configurations and methods for their production are known to those of skill in the art and are disclosed in U.S. Pat. Nos. 5,242,974; 5,384,261; 5,405,783; 5,412,087; 5,424,186; 5,429,807; 5,436,327; 5,445,934; 5,556,752; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,624,711; 5,700,637; 5,744,305; 5,770,456; 5,770,722; 5,837,832; 5,856,101; 5,874,219; 5,885,837; 5,919,523; 6,022,963; 6,077,674; and 6,156,501; Shena, et al., Tibtech 16:301-306, 1998; Duggan, et al., Nat. Genet. 21:10-14, 1999; Bowtell, et al., Nat. Genet. 21:25-32, 1999; Lipshutz, et al., Nature Genet. 21:20-24, 1999; Blanchard, et al., Biosensors and Bioelectronics 11:687-90, 1996; Maskos, et al., Nucleic Acids Res. 21:4663-69, 1993; Hughes, et al., Nat. Biotechnol. 19:342-347, 2001; the disclosures of which are herein incorporated by reference. Patents describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,848,659; and 5,874,219; the disclosures of which are herein incorporated by reference.

In one embodiment, an array of oligonucleotides may be synthesized on a solid support. Exemplary solid supports include glass, plastics, polymers, metals, metalloids, ceramics, organics, etc. Using chip masking technologies and photoprotective chemistry, it is possible to generate ordered arrays of nucleic acid probes. These arrays, which are known, for example, as “DNA chips” or very large scale immobilized polymer arrays (“VLSIPS®” arrays), may include millions of defined probe regions on a substrate having an area of about 1 cm² to several cm², thereby incorporating from a few to millions of probes (see, e.g., U.S. Pat. No. 5,631,734).

To compare expression levels, labeled nucleic acids may be contacted with the array under conditions sufficient for binding between the target nucleic acid and the probe on the array. In one embodiment, the hybridization conditions may be selected to provide for the desired level of hybridization specificity; that is, conditions sufficient for hybridization to occur between the labeled nucleic acids and probes on the microarray.

Hybridization may be carried out in conditions permitting essentially specific hybridization. The length and GC content of the nucleic acid will determine the thermal melting point and thus, the hybridization conditions necessary for obtaining specific hybridization of the probe to the target nucleic acid. These factors are well known to a person of skill in the art, and may also be tested in assays. An extensive guide to nucleic acid hybridization may be found in Tijssen, et al. (Laboratory Techniques in Biochemistry and Molecular Biology, Vol. 24: Hybridization With Nucleic Acid Probes, P. Tijssen, ed.; Elsevier, N.Y. (1993)).

The methods described above will result in the production of hybridization patterns of labeled target nucleic acids on the array surface. The resultant hybridization patterns of labeled nucleic acids may be visualized or detected in a variety of ways, with the particular manner of detection selected based on the particular label of the target nucleic acid. Representative detection means include scintillation counting, autoradiography, fluorescence measurement, calorimetric measurement, light emission measurement, light scattering, and the like.

One such method of detection utilizes an array scanner that is commercially available (Affymetrix, Santa Clara, Calif.), for example, the 417® Arrayer, the 418® Array Scanner, or the Agilent GeneArray® Scanner. This scanner is controlled from a system computer with an interface and easy-to-use software tools. The output may be directly imported into or directly read by a variety of software applications. Exemplary scanning devices are described in, for example, U.S. Pat. Nos. 5,143,854 and 5,424,186.

### Samples for Gene Expression Analysis

In accordance with various embodiments of the invention, cells are analyzed with regard to EMT status. In some embodiments, cancer cells to be analyzed are obtained from a tumor in a cancer patient, such as a patient afflicted with colorectal cancer. The cell sample may be collected in any clinically acceptable manner, provided that the marker-derived polynucleotides (i.e., RNA) are preserved. A cancer cell sample may comprise any clinically relevant tissue sample, such as a tumor biopsy or fine needle aspirate. In some embodiments, the cancer cell sample is obtained from a solid tumor, such as for example, lung cancer, colon cancer, pancreatic cancer, breast cancer, or ovarian cancer.

Nucleic acid specimens may be obtained from the cell sample obtained from a subject to be tested using either “invasive” or “non-invasive” sampling means. A sampling means is said to be “invasive” if it involves the collection of nucleic acids from within the skin or organs of an animal (including murine, human, ovine, equine, bovine, porcine, canine, or feline animal). Examples of invasive methods include, for example, blood collection, semen collection, needle biopsy, pleural aspiration, umbilical cord biopsy. Examples of such methods are discussed by Kim et al. (J. Virol. 66:3879-3882, 1992); Biswas et al. (Ann. NY Acad. Sci. 590:582-583, 1990); and Biswas et al. (J. Clin. Microbiol. 29:2228-2233, 1991).

In one embodiment of the present invention, one or more cells from the subject to be tested are obtained and RNA is isolated from the cells. In one embodiment, a sample of cells is obtained from the subject. It is also possible to obtain a cell sample from a subject, and then to enrich the sample for a desired cell type. For example, cells may be isolated from other cells using a variety of techniques, such as isolation with an antibody binding to an epitope on the cell surface of the desired cell type. Where the desired cells are in a solid tissue, particular cells may be dissected, for example, by microdissection or by laser capture microdissection (LCM) (see, e.g., Bonner, et al., Science 278:1481-1483, 1997; Emmert-Buck, et al., Science 274:998-1001, 1996; Fend, et al., Am. J. Path. 154:61-66, 1999; and Murakami, et al., Kidney Int. 58:1346-1353, 2000).

RNA may be extracted from tissue or cell samples by a variety of methods, for example, guanidium thiocyanate lysis followed by CsCl centrifugation (Chirgwin, et al., Biochemistry 18:5294-5299, 1979). RNA from single cells may be obtained as described in methods for preparing cDNA libraries from single cells (see, e.g., Dulac, Curr. Top. Dev. Biol. 36:245-258, 1998; Jena, et al., J. Immunol. Methods 190:199-213, 1996).

The RNA sample can be further enriched for a particular species. In one embodiment, for example, poly(A)+RNA may be isolated from an RNA sample. In another embodiment, the RNA population may be enriched for sequences of interest by primer-specific cDNA synthesis, or multiple rounds of linear amplification based on cDNA synthesis and template-directed in vitro transcription (see, e.g., Wang, et al., Proc. Natl. Acad. Sci. USA 86:9717-9721, 1989; Dulac, et al., supra; Jena, et al., supra). In addition, the population of RNA, enriched or not, in particular species or sequences, may be further amplified by a variety of amplification methods including, for example, PCR; ligase chain reaction (LCR) (see, e.g., Wu and Wallace, Genomics 4:560-569, 1989; Landegren, et al., Science 241:1077-1080, 1988); self-sustained sequence replication (SSR) (see, e.g., Guatelli, et al., Proc. Natl. Acad. Sci. USA 87:1874-1878, 1990); nucleic acid based sequence amplification (NASBA) and transcription amplification (see, e.g., Kwoh, et al., Proc. Natl. Acad. Sci. USA 86:1173-1177, 1989). Methods for PCR technology are well known in the art (see, e.g., PCR Technology: Principles and Applications for DNA Amplification (ed. H. A. Erlich, Freeman Press, N.Y., N.Y., 1992); PCR Protocols: A Guide to Methods and Applications (eds. Innis, et al., Academic Press, San Diego, Calif., 1990); Mattila, et al., Nucleic Acids Res. 19:4967-4973, 1991; Eckert, et al., PCR Methods and Applications 1:17, 1991; PCR (eds. McPherson et al., IRL Press, Oxford); and U.S. Pat. No. 4,683,202)). Methods of amplification are described, for example, by Ohyama et al. (BioTechniques 29:530-536, 2000); Luo et al. (Nat. Med. 5:117-122, 1999); Hegde et al. (BioTechniques 29:548-562, 2000); Kacharmina et al. (Meth. Enzymol. 303:3-18, 1999); Livesey et al. Curr. Biol. 10:301-310, 2000); Spirin et al. (Invest. Ophthalmol. Vis. Sci. 40:3108-3115, 1999); and Sakai et al. (Anal. Biochem. 287:32-37, 2000). RNA amplification and cDNA synthesis may also be conducted in cells in situ (see, e.g., Eberwine et al., Proc. Natl. Acad. Sci. USA 89:3010-3014, 1992).

### Improving Sensitivity to Expression Level Differences

In using the markers disclosed herein, and, indeed, using any sets of markers to differentiate an individual or subject having one phenotype from another individual or subject having a second phenotype, one can compare the absolute expression of each of the markers in a sample to a control; for example, the control can be the average level of expression of each of the markers, respectively, in a pool of individuals or subjects. To increase the sensitivity of the comparison, however, the expression level values are preferably transformed in a number of ways.

For example, the expression level of each of the biomarkers can be normalized by the average expression level of all markers, the expression level of which is determined, or by the average expression level of a set of control genes. Thus, in one embodiment, the biomarkers are represented by probes on a microarray, and the expression level of each of the biomarkers is normalized by the mean or median expression level across all of the genes represented on the microarray, including any non-biomarker genes. In a specific embodiment, the normalization is carried out by dividing the median or mean level of expression of all of the genes on the microarray. In another embodiment, the expression levels of the biomarkers are normalized by the mean or median level of expression of a set of control biomarkers. In a specific embodiment, the control biomarkers comprise a set of housekeeping genes. In another specific embodiment, the normalization is accomplished by dividing by the median or mean expression level of the control genes.

The sensitivity of a biomarker-based assay will also be increased if the expression levels of individual biomarkers are compared to the expression of the same biomarkers in a pool of samples. Preferably, the comparison is to the mean or median expression level of each the biomarker genes in the pool of samples. Such a comparison may be accomplished, for example, by dividing by the mean or median expression level of the pool for each of the biomarkers from the expression level each of the biomarkers in the sample. This has the effect of accentuating the relative differences in expression between biomarkers in the sample and markers in the pool as a whole, making comparisons more sensitive and more likely to produce meaningful results than the use of absolute expression levels alone. The expression level data may be transformed in any convenient way; preferably, the expression level data for all is log transformed before means or medians are taken.

In performing comparisons to a pool, two approaches may be used. First, the expression levels of the markers in the sample may be compared to the expression level of those markers in the pool, where nucleic acid derived from the sample and nucleic acid derived from the pool are hybridized during the course of a single experiment. Such an approach requires that a new pool of nucleic acid be generated for each comparison or limited numbers of comparisons, and is therefore limited by the amount of nucleic acid available. Alternatively, and preferably, the expression levels in a pool, whether normalized and/or transformed or not, are stored on a computer, or on computer-readable media, to be used in comparisons to the individual expression level data from the sample (i.e., single-channel data).

Thus, the current invention provides the following method of classifying a first cell or subject as having one of at least two different phenotypes, where the different phenotypes comprise a first phenotype and a second phenotype. The level of expression of each of a plurality of genes in a first sample from the first cell or subject is compared to the level of expression of each of said genes, respectively, in a pooled sample from a plurality of cells or subjects, the plurality of cells or subjects comprising different cells or subjects exhibiting said at least two different phenotypes, respectively, to produce a first compared value. The first compared value is then compared to a second compared value, wherein said second compared value is the product of a method comprising comparing the level of expression of each of said genes in a sample from a cell or subject characterized as having said first phenotype to the level of expression of each of said genes, respectively, in the pooled sample. The first compared value is then compared to a third compared value, wherein said third compared value is the product of a method comprising comparing the level of expression of each of the genes in a sample from a cell or subject characterized as having the second phenotype to the level of expression of each of the genes, respectively, in the pooled sample. Optionally, the first compared value can be compared to additional compared values, respectively, where each additional compared value is the product of a method comprising comparing the level of expression of each of said genes in a sample from a cell or subject characterized as having a phenotype different from said first and second phenotypes but included among the at least two different phenotypes, to the level of expression of each of said genes, respectively, in said pooled sample. Finally, a determination is made as to which of said second, third, and, if present, one or more additional compared values, said first compared value is most similar, wherein the first cell or subject is determined to have the phenotype of the cell or subject used to produce said compared value most similar to said first compared value.

In a specific embodiment of this method, the compared values are each ratios of the levels of expression of each of said genes. In another specific embodiment, each of the levels of expression of each of the genes in the pooled sample are normalized prior to any of the comparing steps. In a more specific embodiment, normalization of the levels of expression is carried out by dividing by the median or mean level of the expression of each of the genes or dividing by the mean or median level of expression of one or more housekeeping genes in the pooled sample from said cell or subject. In another specific embodiment, the normalized levels of expression are subjected to a log transform, and the comparing steps comprise subtracting the log transform from the log of the levels of expression of each of the genes in the sample. In another specific embodiment, the two or more different phenotypes relate to the EMT status of the subject sample, i.e., epithelial cell-like or mesenchymal cell-like. In yet another specific embodiment, the levels of expression of each of the genes, respectively, in the pooled sample or said levels of expression of each of said genes in a sample from the cell or subject characterized as having the first phenotype, second phenotype, or said phenotype different from said first and second phenotypes, respectively, are stored on a computer or on a computer-readable medium.

Use of the Markers to Classify a Cancer Patient with Regard to Prognosis

In another aspect, the invention provides a method for classifying a human subject afflicted with a cancer type which is at risk of undergoing an epithelial cell-like to mesenchymal cell-like transition, as having a good prognosis or a poor prognosis. A good prognosis indicates that said subject is expected to have no distant metastases or no reoccurrence within five years of initial diagnosis of said cancer. A poor prognosis indicates that said subject is expected to have distant metastases or a reoccurrence of cancer within five years of initial diagnosis of said cancer. The method according to this aspect of the invention comprises: (a) classifying cancer cells obtained from said human subject as having mesenchymal cell-like qualities or epithelial cell-like qualities on the basis of levels of the expression level of at least five of the genes for which markers are listed in one or more of TABLE 2A, TABLE 2B, TABLE 4A, TABLE 4B, TABLE 9A, and TABLE 9B; and (b) classifying the human subject as having a good prognosis if the cancer cells are classified according to step (a) as having epithelial cell-like properties, or classifying the human subject as having a poor prognosis if the cancer cells are classified according to step (a) as having mesenchymal cell-like properties. The methods of this aspect of the invention may be carried out on a suitably programmed computer, and optionally may be displayed; or output to a user, user interface device, a computer readable storage medium, or a local or remote computer system.

The classification of the cancer cells as having mesenchymal cell-like qualities or epithelial cell-like qualities may be carried out using classification methods as described herein.

In some embodiments, the expression levels of the mesenchymal arm genes (for which markers are provided in TABLE 2A) and/or the epithelial arm genes (for which markers are provided in TABLE 2B) are used to calculate an Epithelial to Mesenchymal Transition (EMT) signature score for a cancer cell, or population of cancer cells. In other embodiments of the invention, the expression levels of the mesenchymal arm genes (for which markers are provided in TABLE 4A) and/or the epithelial arm genes (for which markers are provided in TABLE 4B) are used to calculate a PC1 (first principal component) signature score for a cancer cell, or a plurality of cancer cells.

In one embodiment, the method comprises calculating an EMT Signature Score for the cancer cells isolated from the human subject by a method comprising: (i) calculating a differential expression value of a first expression level of each of a first plurality of genes and each of a second plurality of genes in the isolated cancer cell sample derived from the human subject relative to a second expression level of each of said first plurality of genes and each of said second plurality of genes in a human control cell sample, said first plurality of genes consisting of at least 5 or more of the genes for which markers are listed in one or more of TABLES 2A, 4A, and 9A (mesenchymal Arm) and said second plurality of genes consisting of at least 5 or more of the genes for which markers are listed in one or more of TABLES 2B, 4B, and 9B (epithelial Arm); (ii) calculating the mean differential expression values of the expression levels of said first plurality of genes and said second plurality of genes; (iii) subtracting said mean differential expression value of said second plurality of genes from said mean differential expression value of said first plurality of genes to obtain said EMT Signature score; and (iv) classifying said cancer cell sample as having mesenchymal cell-like properties if said obtained EMT Signature score is at or above a first predetermined threshold and is statistically significant; or classifying said cancer cell sample as having epithelial cell-like properties if said obtained EMT Signature score is at or below a second predetermined threshold and is statistically significant.

In one embodiment, said first plurality of genes consists of at least 6, 7, 8, 9, or 10, or more of the genes for which markers are listed in TABLE 2A. In one embodiment, said second plurality of genes consists of at least 6, 7, 8, 9, or 10, or more of the genes for which markers are listed in TABLE 2B. In one embodiment, said first plurality of genes consists of at least 11, 12, 13, 14, 15, 16, 17, 18, 19, or 20, or more of the genes for which markers are listed in TABLE 2A. In one embodiment, said second plurality of genes consists of at least 11, 12, 13, 14, 15, 16, 17, 18, 19, or 20, or more of the genes for which markers are listed in TABLE 2B. In one embodiment, said first plurality of genes consists of at least 21, 22, 23, 24, 25, 26, 27, 28, 29, or 30, or more of the genes for which markers are listed in TABLE 2A. In one embodiment, said second plurality of genes consists of at least 21, 22, 23, 24, 25, 26, 27, 28, 29, or 30, or more genes for which markers are listed in TABLE 2B. In one embodiment, said first plurality of genes consists of all of the genes for which markers are listed in TABLE 2A. In one embodiment, said second plurality of genes consists of all of the genes for which markers are listed in TABLE 2B.

In one embodiment, said differential expression value is log(10) ratio. In one embodiment, said first and second predetermined threshold is 0. In one embodiment, said first predetermined threshold is from 0.1 to 0.3. In one embodiment, said second predetermined threshold is from ⁻0.1 to ⁻0.3. In one embodiment, said EMT Signature Score is statistically significant if it has a p-value less than 0.05.

In some embodiments, the methods according to this aspect of the invention are used to classify a human subject suffering from a cancer type that is at risk for undergoing an epithelial cell-like to mesenchymal cell-like transition, such as, for example, colon cancer, lung cancer, pancreatic cancer, breast cancer, ovarian cancer or prostate cancer.

Poor prognosis of a cancer, such as colon cancer, may indicate that a tumor is relatively aggressive, while a good prognosis may indicate that the tumor is relatively non-aggressive. Therefore, in another embodiment, the invention provides for a method of determining a course of treatment of a cancer patient, such as a colon cancer patient, comprising determining EMT status of cancer cells obtained from the patient, wherein if the cancer cells are classified as having mesenchymal cell-like properties (i.e., a poor prognosis), the tumor is treated as an aggressive tumor.

### Kits and Computer-Facilitated Data Analysis

The present invention further provides for kits for carrying out the various embodiments of the methods of the invention, wherein the kits comprise the various embodiments of the EMT and/or PC1 signature marker sets described herein.

In one embodiment, the invention provides a kit for predicting the response of a human subject with cancer to a treatment that induces a therapeutically beneficial response in cancer cells having epithelial cell-like qualities, wherein the kit comprises

PCR primers and/or probes for measuring the gene expression level of at least 5 of the genes for which markers are listed in any of TABLES 2A, TABLE 2B, TABLE 4A, TABLE 4B, TABLE 9A and TABLE 9B. In one embodiment, the kit comprises PCR primers and/or probes for measuring at least 5 of the genes listed in TABLE 2A and TABLE 2B. In one embodiment, the kit comprises PCR primers and/or probes for measuring at least 5 of the genes listed in TABLE 4A and TABLE 4B. In one embodiment, the kit comprises PCR primers and/or probes for measuring the expression level of one or more of the microRNAs listed in TABLE 9A (SEQ ID NO:509-582) and/or TABLE 9B (SEQ ID NO:583-639). In one embodiment, the kit comprises at least 5 of the cDNA probes listed in TABLE 2A (SEQ ID NOS:1-149) and/or TABLE 2B (SEQ ID NOS: 150-310).

In another embodiment, the invention provides a kit for classifying a human subject afflicted with a cancer type which is at risk for undergoing an epithelial cell-like to mesenchymal cell-like transition as having a good prognosis or a poor prognosis, wherein the kit comprises reagents for classifying cancer cells obtained from said human subject as having mesenchymal cell-like qualities or epithelial cell-like qualities, wherein the reagents comprise PCR primers and/or probes for measuring the gene expression level of at least 5 of the genes for which markers are listed in any of TABLE 2A, TABLE 2B, TABLE 4A, TABLE 4B, TABLE 9A and TABLE 9B. In one embodiment, the kit comprises PCR primers and/or probes for measuring at least 5 of the genes listed in TABLE 2A and TABLE 2B. In one embodiment, the kit comprises PCR primers and/or probes for measuring at least 5 of the genes listed in TABLE 4A and TABLE 4B. In one embodiment, the kit comprises PCR primers and/or probes for measuring the expression level of one or more of the microRNAs listed in TABLE 9A (SEQ ID NO:509-582) and/or TABLE 9B (SEQ ID NO:583-639). In one embodiment, the kit comprises at least 5 of the cDNA probes listed in TABLE 2A (SEQ ID NOS:1-149) and/or TABLE 2B (SEQ ID NOS: 150-310).

In some embodiments, the kit contains a microarray ready for hybridization to target polynucleotide molecules prepared from a sample to be evaluated, plus software for the data analyses described above. In another embodiment, the kit contains a set of PCR primer pairs for a plurality of the EMT and/or PC1 signature biomarker genes that are ready for hybridization to target polynucleotide molecules prepared from a sample to be evaluated, plus software for the data analyses described herein.

A kit of the invention can also provide reagents for primer extension and amplification reactions. For example, in some embodiments, the kit may further include one or more of the following components: a reverse transcriptase enzyme, a DNA polymerase enzyme, a Tris buffer, a potassium salt (e.g., potassium chloride), a magnesium salt (e.g., magnesium chloride), a reducing agent (e.g., dithiothreitol), and dNTPs.

The analytic methods described in the previous sections can be implemented by use of kits and the following computer systems and according to the following programs and methods. A computer system comprises internal components linked to external components. The internal components of a typical computer system include a processor element interconnected with a main memory. For example, the computer system can be an Intel 8086-, 80386-, 80486-, Pentium®, or Pentium®-based processor with preferably 32 MB or more of main memory.

The external components may include mass storage. This mass storage can be one or more hard disks (which are typically packaged together with the processor and memory). Such hard disks are preferably of 1 GB or greater storage capacity. Other external components include a user interface device, which can be a monitor, together with an inputting device, which can be a “mouse,” or other graphic input devices, and/or a keyboard. A printing device can also be attached to the computer.

Typically, a computer system is also linked to a network, which can be part of an Ethernet linked to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on the mass storage device. A software component comprises the operating system, which is responsible for managing the computer system and its network interconnections. This operating system can be, for example, of the Microsoft Windows® family, such as Windows 3.1, Windows 95, Windows 98, Windows 2000, or Windows NT. The software component represents common languages and functions conveniently present on this system to assist programs implementing the methods specific to this invention. Many high or low level computer languages can be used to program the analytic methods of this invention. Instructions can be interpreted during run-time or compiled. Preferred languages include C/C++, FORTRAN and JAVA. Most preferably, the methods of this invention are programmed in mathematical software packages that allow symbolic entry of equations and high-level specification of processing, including some or all of the algorithms to be used, thereby freeing a user of the need to procedurally program individual equations or algorithms. Such packages include Mathlab from Mathworks (Natick, Mass.), Mathematica® from Wolfram Research (Champaign, Ill.), or S-Plus®D from Math Soft (Cambridge, Mass.). Specifically, the software component includes the analytic methods of the invention as programmed in a procedural language or symbolic package.

The software to be included with the kit comprises the data analysis methods of the invention as disclosed herein. In particular, the software may include mathematical routines for biomarker discovery, including the calculation of correlation coefficients between clinical categories (i.e., response to cancer therapy agents) and biomarker gene expression levels. The software may also include mathematical routines for calculating the correlation between sample EMT biomarker expression and control EMT biomarker expression, using, for example, array-generated fluorescence data or PCR amplification levels, to determine the clinical classification of a sample.

In an exemplary implementation, to practice the methods of the present invention, a user first loads data indicative of EMT and/or PC1 biomarker expression levels into the computer system. These data can be directly entered by the user from a monitor, keyboard, or from other computer systems linked by a network connection, or on removable storage media such as a CD-ROM, floppy disk (not illustrated), tape drive (not illustrated), ZIP® drive (not illustrated), or through the network. Next, the user causes execution of EMT and/or PC1 expression profile analysis software which performs the methods of the present invention.

In another exemplary implementation, a user first loads experimental data and/or databases into the computer system. This data is loaded into the memory from the storage media or from a remote computer, preferably from a dynamic gene set database system, through the network. Next the user causes execution of software that performs the steps of the present invention.

Alternative computer systems and software for implementing the analytic methods of this invention will be apparent to one of skill in the art and are intended to be comprehended within the accompanying claims. In particular, the accompanying claims are intended to include the alternative program structures for implementing the methods of this invention that will be readily apparent to one of skill in the art.

The following examples merely illustrate the best mode now contemplated for practicing the invention, but should not be construed to limit the invention.

### EXAMPLES

### Example 1

### Identification of a Lung Cancer Cell Line Derived EMT Gene Expression Signature that Classifies Epithelial Cell-like Cancer Samples from Mesenchymal Cell-like Samples

### Methods:

Candidate genes for an EMT biomarker signature were identified by performing a t-test using a microarray dataset obtained from 93 lung cancer cell lines comparing cell lines exhibiting mesenchymal-like gene expression pattern (i.e., high levels of VIM gene expression and low levels of CDH1 gene expression) vs. cell lines with epithelial-like gene expression pattern (low levels of VIM gene expression and high levels of CDH1 gene expression). Vimentin (VIM), GenBank ref. NM_{—}003380, set forth as SEQ ID NO:122. Epithelial cadherin type 1 (CDH1), GenBank ref. NM_{—}004360, set forth as SEQ ID NO:222.

Cell samples from each of the 93 human lung cancer cell lines listed in TABLE 1 were gene expression profiled using a human microarray. Nucleic acid was purified from the cell samples, amplified and hybridized onto Merck custom human array 1.0 chip (GPL6793/GPL10687), manufactured by Affymetrix Inc, Santa Clara Calif., following standard Affymetrix protocols.

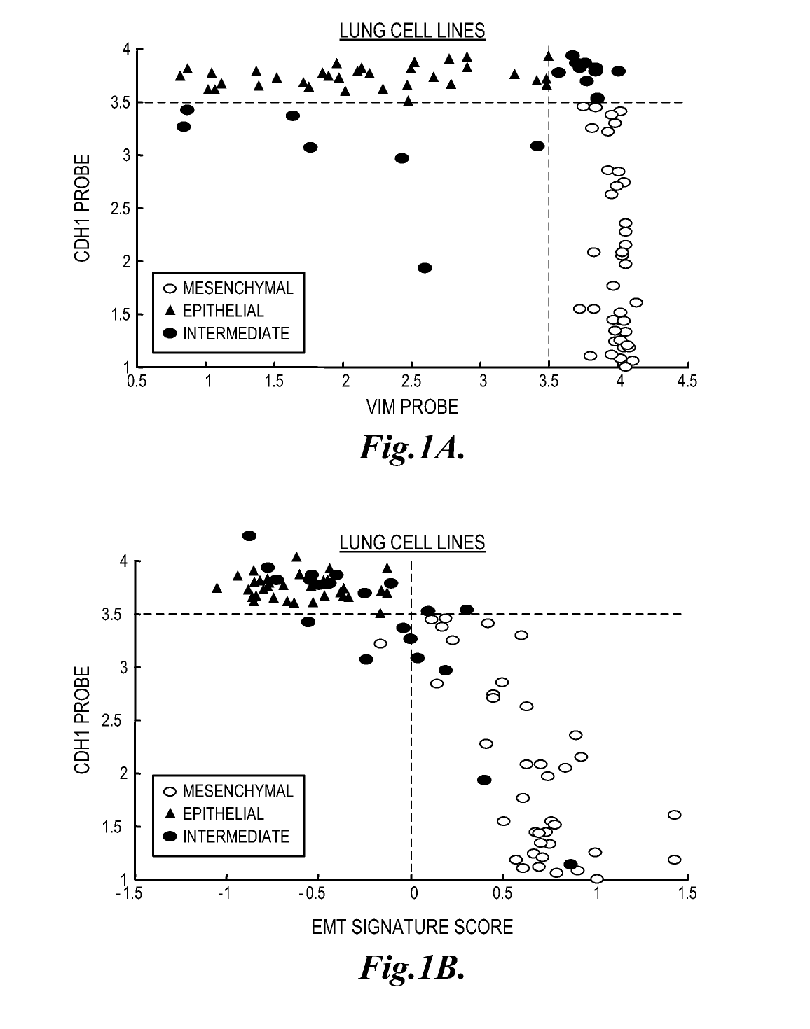

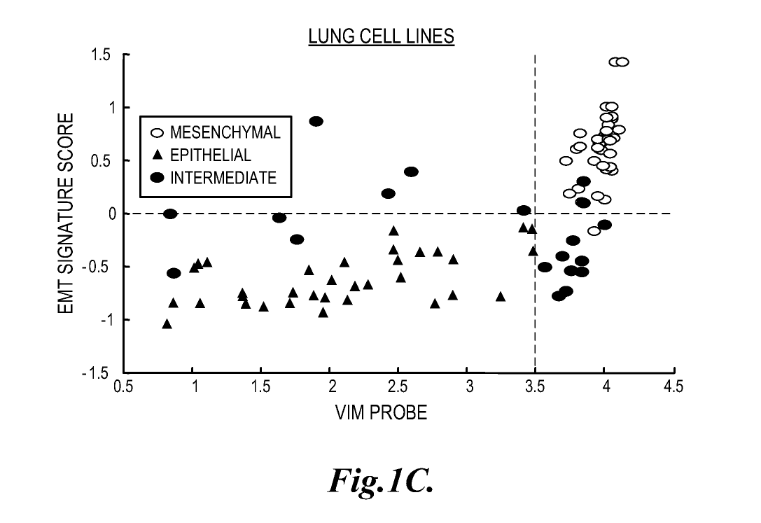

The 93 lung cancer cell lines were then divided into three groups based on the resulting gene expression profiles (FIG. 1A). FIG. 1A shows a plot of the 93 lung cancer cell lines distributed by CDH1 gene expression level (y-axis) versus VIM gene expression level (x-axis). As shown in FIG. 1A, a first group of lung cancer cell lines was defined as having similarity to epithelial cells (i.e., exhibited a high level of CDH1 gene expression, and a low level of VIM gene expression). A second group of lung cancer cell lines was defined as having similarity to mesenchymal cells (i.e., exhibited a low level of CDH1 gene expression and a high level of VIM gene expression). A third group of lung cancer cell lines was designated as intermediate (i.e., these cell lines had CDH1 and VIM gene expression values that were either each less than 3.5 (eight cell lines) or were above 3.5 for both genes (eleven cell lines)) (see FIG. 1, Panel A). Probe intensities were measured following standard Robust Multi-Array Average (RMA) procedure, and reported in dimensionless units.

TABLE 1
List of 93 Lung Tumor Cell Lines.

| | | | CDH1 | |
|---|---|---|---|---|
| | | VIM | Expres- | EMT |
| Lung Tumor Cell | Classification | Expression | sion | Signature |
| Line Name | Group | Level | Level | Score |
| 39 Mesenchymal | | | | |
| cell-like lung | | | | |
| tumor cell lines | | | | |
| HLFa | Mesenchymal | 4.07 | 1.19 | 1.34 |
| Hs573.T | Mesenchymal | 4.12 | 1.61 | 1.34 |
| MSTO-211H | Mesenchymal | 4.05 | 1.00 | 0.95 |
| H2052 | Mesenchymal | 4.01 | 1.25 | 0.93 |
| H2122 | Mesenchymal | 4.04 | 2.16 | 0.86 |
| H2452 | Mesenchymal | 4.01 | 1.09 | 0.85 |
| CALU-1 | Mesenchymal | 4.05 | 2.36 | 0.84 |
| H1792 | Mesenchymal | 4.03 | 2.05 | 0.78 |
| LU99A | Mesenchymal | 4.09 | 1.06 | 0.74 |
| LXF289 | Mesenchymal | 4.00 | 1.52 | 0.72 |
| H1299 | Mesenchymal | 4.04 | 1.34 | 0.72 |
| H1563 | Mesenchymal | 3.82 | 1.55 | 0.71 |
| H661 | Mesenchymal | 4.05 | 1.97 | 0.70 |
| H1703 | Mesenchymal | 3.99 | 1.45 | 0.70 |
| LCLC103H | Mesenchymal | 4.06 | 1.21 | 0.67 |
| H1915 | Mesenchymal | 3.97 | 1.35 | 0.67 |
| SW1573 | Mesenchymal | 4.03 | 1.43 | 0.66 |
| H460 | Mesenchymal | 3.95 | 1.12 | 0.66 |
| SKMES1 | Mesenchymal | 4.02 | 2.09 | 0.65 |
| COLO-699N | Mesenchymal | 3.97 | 1.24 | 0.63 |
| H226 | Mesenchymal | 3.95 | 1.45 | 0.63 |
| H2172 | Mesenchymal | 3.82 | 2.09 | 0.60 |
| COLO699 | Mesenchymal | 3.79 | 1.11 | 0.59 |
| RERF_LC_MS | Mesenchymal | 3.95 | 2.63 | 0.58 |
| H2030 | Mesenchymal | 3.95 | 1.76 | 0.58 |
| H23 | Mesenchymal | 3.97 | 3.30 | 0.57 |
| H28 | Mesenchymal | 4.04 | 1.19 | 0.54 |
| H522 | Mesenchymal | 3.72 | 1.55 | 0.49 |
| A549 | Mesenchymal | 3.91 | 2.85 | 0.46 |
| HCC44 | Mesenchymal | 3.99 | 2.72 | 0.42 |
| H647 | Mesenchymal | 4.03 | 2.74 | 0.41 |
| H1755 | Mesenchymal | 4.01 | 3.41 | 0.39 |
| A427 | Mesenchymal | 4.05 | 2.28 | 0.39 |
| H1793 | Mesenchymal | 3.80 | 3.26 | 0.21 |
| H2023 | Mesenchymal | 3.74 | 3.46 | 0.18 |
| HCC15 | Mesenchymal | 3.94 | 3.38 | 0.16 |
| H2228 | Mesenchymal | 3.99 | 2.84 | 0.12 |
| H596 | Mesenchymal | 3.82 | 3.45 | 0.10 |
| H2073 | Mesenchymal | 3.91 | 3.22 | −0.15 |
| 35 Epithelial cell- | | | | |
| like lung tumor | | | | |
| cell lines | | | | |
| H1650 | Epithelial | 3.49 | 3.92 | −0.13 |
| H1944 | Epithelial | 3.47 | 3.71 | −0.14 |
| H1693 | Epithelial | 3.40 | 3.70 | −0.15 |
| CORL_105 | Epithelial | 2.47 | 3.50 | −0.16 |
| HARA | Epithelial | 2.46 | 3.66 | −0.33 |
| H1838 | Epithelial | 2.65 | 3.73 | −0.34 |
| HARA_B | Epithelial | 2.79 | 3.67 | −0.34 |
| H1734 | Epithelial | 3.47 | 3.67 | −0.35 |
| H1568 | Epithelial | 2.48 | 3.82 | −0.43 |
| RERF_LC_ad2 | Epithelial | 2.90 | 3.92 | −0.43 |
| UMC-11 | Epithelial | 1.11 | 3.67 | −0.44 |
| H292 | Epithelial | 2.11 | 3.79 | −0.45 |
| CHAGO-K-1 | Epithelial | 1.05 | 3.77 | −0.46 |
| COLO_668 | Epithelial | 1.01 | 3.61 | −0.50 |
| CAL12T | Epithelial | 1.85 | 3.77 | −0.51 |
| KNS62 | Epithelial | 2.52 | 3.87 | −0.59 |
| H1993 | Epithelial | 2.01 | 3.60 | −0.60 |
| H1666 | Epithelial | 2.28 | 3.62 | −0.64 |
| H727 | Epithelial | 2.18 | 3.76 | −0.65 |
| CORL23/R | Epithelial | 1.74 | 3.65 | −0.71 |
| HCC827 | Epithelial | 2.90 | 3.83 | −0.73 |
| LUDLU1 | Epithelial | 1.36 | 3.78 | −0.73 |
| HCC78 | Epithelial | 3.24 | 3.76 | −0.75 |
| H1573 | Epithelial | 1.36 | 3.79 | −0.75 |
| CORL-23/CPR | Epithelial | 1.97 | 3.72 | −0.75 |
| H1648 | Epithelial | 1.88 | 3.75 | −0.75 |
| H2342 | Epithelial | 2.13 | 3.81 | −0.78 |
| H2170 | Epithelial | 0.86 | 3.80 | −0.79 |
| CORL23 | Epithelial | 1.70 | 3.66 | −0.80 |
| DV90 | Epithelial | 1.39 | 3.65 | −0.80 |
| H1437 | Epithelial | 1.06 | 3.61 | −0.81 |
| H1869 | Epithelial | 2.77 | 3.90 | −0.81 |
| CORL23/R23- | Epithelial | 1.52 | 3.72 | −0.83 |
| H441 | Epithelial | 1.95 | 3.86 | −0.88 |
| H2126 | Epithelial | 0.81 | 3.74 | −1.00 |
| 19 Intermediate | | | | |
| lung tumor cell | | | | |
| lines | | | | |
| SKLU1 | Intermediate | 1.89 | 1.14 | 0.82 |
| H1155 | Intermediate | 2.59 | 1.94 | 0.38 |
| H1651 | Intermediate | 3.84 | 3.54 | 0.28 |
| HCC 366 | Intermediate | 2.43 | 2.97 | 0.17 |
| H2085 | Intermediate | 3.84 | 3.53 | 0.08 |
| H520 | Intermediate | 3.41 | 3.09 | 0.04 |
| H2106 | Intermediate | 0.83 | 3.27 | 0.01 |
| LK2 | Intermediate | 1.63 | 3.36 | −0.04 |
| H2444 | Intermediate | 3.99 | 3.79 | −0.12 |
| PC7 | Intermediate | 1.76 | 3.07 | −0.21 |
| EPLC_272H | Intermediate | 3.77 | 3.70 | −0.25 |
| H2009 | Intermediate | 3.69 | 3.86 | −0.39 |
| H1975 | Intermediate | 3.83 | 3.79 | −0.42 |
| HCC4006 | Intermediate | 3.55 | 3.78 | −0.48 |
| EBC1 | Intermediate | 3.75 | 3.87 | −0.51 |
| H2347 | Intermediate | 3.83 | 3.82 | −0.52 |
| H1395 | Intermediate | 0.86 | 3.42 | −0.52 |
| CALU3 | Intermediate | 3.72 | 3.82 | −0.70 |
| H358 | Intermediate | 3.67 | 3.94 | −0.73 |

Genes that were selected with a VIM or CDH1 classification value with p-value <0.01 by the t-test were split into two groups: the mesenchymal arm or “up arm” and the epithelial arm or “down arm”. TABLE 2A lists the 149 gene markers in the mesenchymal arm (“up arm”) that were found to be up-regulated in the lung cancer cell lines that were classified as mesenchymal cell-like, as compared to the lung cancer cell lines that were classified as epithelial cell-like, and were also found to be down-regulated in the lung tumor cell lines that were classified as epithelial cell-like as compared to the lung cancer cell lines that were classified as mesenchymal cell-like. TABLE 2A provides for each of the 149 gene markers, the gene symbol; the Genbank reference number for each gene symbol as of Oct. 1, 2010, each of which is hereby incorporated herein by reference; and the SEQ ID NO: corresponding to an exemplary 60-mer sequence that corresponds to a portion of the corresponding cDNA, which may be used as a probe.

TABLE 2A
149 EMT Signature Genes: The Mesenchymal or
Up-Regulated Arm.

| | Gene Transcript | |
|---|---|---|
| | Genbank | Transcript |
| Gene | reference | probe SEQ |
| Symbol | Number | ID NO: |
| FAM171A1 | AY683003 | 1 |
| ZCCHC24 | BC028617 | 2 |
| GLIPR2 | AK091288 | 3 |
| TMSB15A | BG471140 | 4 |
| COL12A1 | NM_004370 | 5 |
| LOX | NM_002317 | 6 |
| SPARC | AK126525 | 7 |
| CDH11 | D21255 | 8 |
| ZEB1 | BX647794 | 9 |
| EML1 | NM_001008707 | 10 |
| ZNF788 | AK128700 | 11 |
| WIPF1 | NM_001077269 | 12 |
| CAP2 | NM_006366 | 13 |
| TGFB2 | AB209842 | 14 |
| DLC1 | NM_182643 | 15 |
| POSTN | NM_006475 | 16 |
| NEGR1 | NM_173808 | 17 |
| JAM3 | AK027435 | 18 |
| SRPX | BC020684 | 19 |
| BICC1 | NM_001080512 | 20 |
| HAS2 | NM_005328 | 21 |
| ANTXR1 | NM_032208 | 22 |
| GNB4 | NM_021629 | 23 |
| COL4A1 | NM_001845 | 24 |
| SRGN | CD359027 | 25 |
| SUSD5 | NM_015551 | 26 |
| DIO2 | NM_013989 | 27 |
| GLIPR1 | NM_006851 | 28 |
| COL5A1 | NM_000093 | 29 |
| NAP1L3 | BC094729 | 30 |
| RBMS3 | BQ214991 | 31 |
| BVES | BC040502 | 32 |
| SLC47A1 | BC010661 | 33 |
| FGFR1 | NM_023110 | 34 |
| FSTL1 | NM_007085 | 35 |
| FGF2 | NM_002006 | 36 |
| DKK3 | NM_015881 | 37 |
| CMTM3 | AK056324 | 38 |
| PTGIS | NM_000961 | 39 |
| CCL2 | BU570769 | 40 |
| WNT5B | BC001749 | 41 |
| CLDN11 | AK098766 | 42 |
| MAP1B | NM_005909 | 43 |
| IL13RA2 | AK308523 | 44 |
| MSRB3 | NM_001031679 | 45 |
| FAM101B | AK093557 | 46 |
| ZEB2 | NM_014795 | 47 |
| NID1 | NM_002508 | 48 |
| TMEM158 | NM_015444 | 49 |
| ST3GAL2 | AK127322 | 50 |
| FGF5 | NM_004464 | 51 |
| AKAP12 | NM_005100 | 52 |
| GPR176 | BC067106 | 53 |
| PMP22 | NM_000304 | 54 |
| LEPREL1 | NM_018192 | 55 |
| CHN1 | NM_001822 | 56 |
| TTC28 | NM_001145418 | 57 |
| GLT25D2 | NM_015101 | 58 |
| RECK | BX648668 | 59 |
| GREM1 | NM_013372 | 60 |
| C16orf45 | AK092923 | 61 |
| AOX1 | L11005 | 62 |
| CTGF | NM_001901 | 63 |
| ANXA6 | NM_001155 | 64 |
| SERPINE1 | NM_000602 | 65 |
| SLC2A3 | AB209607 | 66 |
| ZFPM2 | NM_012082 | 67 |
| FHL1 | NM_001159704 | 68 |
| ATP8B2 | NM_020452 | 69 |
| RBPMS2 | AY369207 | 70 |
| TBXA2R | NM_001060 | 71 |
| COL3A1 | NM_000090 | 72 |
| GPC6 | NM_005708 | 73 |
| AFF3 | NM_002285 | 74 |
| PLAGL1 | CR749329 | 75 |
| LGALS1 | BF570935 | 76 |
| TTLL7 | NM_024686 | 77 |
| COL5A2 | NM_000393 | 78 |
| ANKRD1 | NM_014391 | 79 |
| NRG1 | NM_013960 | 80 |
| POPDC3 | NM_022361 | 81 |
| C1S | NM_201442 | 82 |
| CDH2 | NM_001792 | 83 |
| DOCK10 | NM_014689 | 84 |
| CLIP3 | AK094738 | 85 |
| CDH4 | AL834206 | 86 |
| COL6A1 | NM_001848 | 87 |
| HEG1 | NM_020733 | 88 |
| IGFBP7 | BX648756 | 89 |
| DAB2 | NM_001343 | 90 |
| F2R | NM_001992 | 91 |
| EDIL3 | BX648583 | 92 |
| COL1A2 | J03464 | 93 |
| HTRA1 | NM_002775 | 94 |
| NDN | NM_002487 | 95 |
| BDNF | EF689009 | 96 |
| LHFP | NM_005780 | 97 |
| PRKD1 | X75756 | 98 |
| MMP2 | NM_004530 | 99 |
| UCHL1 | AB209038 | 100 |
| DPYSL3 | BC077077 | 101 |
| RBM24 | AL832199 | 102 |
| DFNA5 | AK094714 | 103 |
| MRAS | NM_012219 | 104 |
| SYDE1 | AK128870 | 105 |
| FLRT2 | NM_013231 | 106 |
| AK5 | NM_012093 | 107 |
| EPDR1 | XM_002342700 | 108 |
| TUB | NM_003320 | 109 |
| SIRPA | NM_001040022 | 110 |
| AXL | NM_021913 | 111 |
| FBN1 | NM_000138 | 112 |
| EVI2A | NM_001003927 | 113 |
| PTX3 | NM_002852 | 114 |
| ADAM23 | AK091800 | 115 |
| PNMA2 | NM_007257 | 116 |
| PDE7B | AB209990 | 117 |
| TCF4 | NM_001083962 | 118 |
| KIRREL | AK090554 | 119 |
| NEXN | NM_144573 | 120 |
| ALPK2 | BX647796 | 121 |
| VIM | NM_003380 | 122 |
| LIX1L | AK128733 | 123 |
| ADAMTS1 | NM_006988 | 124 |
| PAPPA | NM_002581 | 125 |
| ANGPTL2 | NM_012098 | 126 |
| AP1S2 | BX647483 | 127 |
| TUBA1A | BI083878 | 128 |
| LAMA4 | NM_001105206 | 129 |
| EPB41L5 | BC054508 | 130 |
| NAV3 | NM_014903 | 131 |
| ELOVL2 | BC050278 | 132 |
| BNC2 | NM_017637 | 133 |
| GFPT2 | BC000012 | 134 |
| TRPA1 | Y10601 | 135 |
| PRR16 | AF242769 | 136 |
| CYBRD1 | NM_024843 | 137 |
| HS3ST3A1 | NM_006042 | 138 |
| GNG11 | BF971151 | 139 |
| TMEM47 | BC039242 | 140 |
| CPA4 | NM_016352 | 141 |
| ARMCX1 | CR933662 | 142 |
| RFTN1 | NM_015150 | 143 |
| EMP3 | BM556279 | 144 |
| ATP8B3 | AK125969 | 145 |
| FAT4 | NM_024582 | 146 |
| NUDT11 | NM_018159 | 147 |
| PTRF | NM_012232 | 148 |
| TNFRSF19 | NM_148957 | 149 |

TABLE 2B lists the 161 gene markers in the epithelial arm (“down arm”) that were found to be down-regulated in the lung tumor cell lines that were classified as mesenchymal cell-like, as compared to the lung cancer cell lines that were classified as epithelial cell-like, and were also found to be up-regulated in the lung cancer cell lines that were classified as epithelial cell-like as compared to the lung cancer cell lines that were classified as mesenchymal cell-like. TABLE 2B provides for each of the 161 gene markers, the gene symbol; the Genbank reference number for each gene symbol as of Oct. 1, 2010, each of which is hereby incorporated herein by reference; and the SEQ ID NO: corresponding to an exemplary 60-mer sequence that corresponds to a portion of the corresponding cDNA, which may be used as a probe.

TABLE 2B
161 EMT Signature Genes: The Epithelial or
Down-Regulated Arm.

| | Gene Transcript | Transcript |
|---|---|---|
| | Genbank | probe SEQ |
| Gene Symbol | Reference No. | ID NO: |
| PRR15L | BC002865 | 150 |
| TTC39A | AB007921 | 151 |
| ESRP1 | NM_017697 | 152 |
| RBM35B | CR607695 | 153 |
| AGR3 | BG540617 | 154 |
| TMEM125 | BC072393 | 155 |
| KLK8 | DQ267420 | 156 |
| MBNL3 | NM_001170704 | 157 |
| SPRR1B | AI541215 | 158 |
| S100A9 | BQ927179 | 159 |
| TMC5 | NM_001105248 | 160 |
| ELF5 | NM_198381 | 161 |
| ERBB3 | NM_001982 | 162 |
| WDR72 | NM_182758 | 163 |
| FAM84B | NM_174911 | 164 |
| SPRR3 | EF553525 | 165 |
| TMEM30B | NM_001017970 | 166 |
| C1orf210 | NM_182517 | 167 |
| TMPRSS4 | NM_019894 | 168 |
| ERP27 | BC030218 | 169 |
| TTC22 | NM_017904 | 170 |
| CNKSR1 | BC012797 | 171 |
| FGFBP1 | NM_005130 | 172 |
| FUT3 | NM_000149 | 173 |
| GALNT3 | NM_004482 | 174 |
| RAPGEF5 | NM_012294 | 175 |
| MAPK13 | AB209586 | 176 |
| AP1M2 | BC005021 | 177 |
| CDH3 | NM_001793 | 178 |
| PPL | NM_002705 | 179 |
| GCNT3 | EF152283 | 180 |
| EPPK1 | AB051895 | 181 |
| MAL2 | NM_052886 | 182 |
| TMPRSS11E | NM_014058 | 183 |
| LCN2 | AK307311 | 184 |
| ANKRD22 | NM_144590 | 185 |
| POU2F3 | AF162715 | 186 |
| SPINT1 | BC018702 | 187 |
| AQP3 | NM_004925 | 188 |
| GPR110 | CR627234 | 189 |
| FAM84A | NM_145175 | 190 |
| TMPRSS13 | NM_001077263 | 191 |
| GPX2 | BE512691 | 192 |
| WFDC2 | BM921431 | 193 |
| KLK10 | NM_002776 | 194 |
| S100A14 | BG674026 | 195 |
| S100P | BG571732 | 196 |
| FXYD3 | BF676327 | 197 |
| MUC20 | XR_078298 | 198 |
| SPINT2 | NM_021102 | 199 |
| C1orf116 | NM_023938 | 200 |
| SPINK5 | NM_001127698 | 201 |
| ANXA9 | NM_003568 | 202 |
| TMC4 | NM_001145303 | 203 |
| SYK | NM_003177 | 204 |
| HOOK1 | NM_015888 | 205 |
| FAM83A | DQ280323 | 206 |
| LCP1 | NM_002298 | 207 |
| HS6ST2 | NM_001077188 | 208 |
| TSPAN1 | NM_005727 | 209 |
| S100A8 | BG739729 | 210 |
| DMKN | BC035311 | 211 |
| GRHL1 | NM_198182 | 212 |
| CKMT1B | AK094322 | 213 |
| ACPP | NM_001099 | 214 |
| PTAFR | NM_000952 | 215 |
| KRT5 | M21389 | 216 |
| DAPP1 | NM_014395 | 217 |
| LAMA3 | NM_198129 | 218 |
| C19orf21 | NM_173481 | 219 |
| SH2D3A | AK024368 | 220 |
| TOX3 | AK095095 | 221 |
| CDH1 | NM_004360 | 222 |
| FA2H | NM_024306 | 223 |
| SPRR1A | NM_005987 | 224 |
| LIPG | BC060825 | 225 |
| CEACAM6 | NM_002483 | 226 |
| PROM2 | NM_001165978 | 227 |
| ITGB6 | AL831998 | 228 |
| OR2A4 | BC120953 | 229 |
| MAP7 | NM_003980 | 230 |
| PPP1R14C | AF407165 | 231 |
| PVRL4 | NM_030916 | 232 |
| FBP1 | NM_000507 | 233 |
| FAAH2 | NM_174912 | 234 |
| LAMB3 | NM_001017402 | 235 |
| MPP7 | NM_173496 | 236 |
| ANK3 | NM_020987 | 237 |
| SYT7 | NM_004200 | 238 |
| TRIM29 | BX648072 | 239 |
| TMEM45B | AK098106 | 240 |
| ST14 | NM_021978 | 241 |
| ARHGDIB | AK125625 | 242 |
| HS3ST1 | AK096823 | 243 |
| KLK5 | AY359010 | 244 |
| GJB6 | NM_001110219 | 245 |
| CCDC64B | NM_001103175 | 246 |
| PAK6 | AK131522 | 247 |
| MARVELD3 | NM_001017967 | 248 |
| CLDN7 | NM_001307 | 249 |
| SH3YL1 | AK123829 | 250 |
| SLPI | BG483345 | 251 |
| MB | BF670653 | 252 |
| NPNT | NM_001033047 | 253 |
| C1orf106 | NM_001142569 | 254 |
| DSP | NM_004415 | 255 |
| STEAP4 | NM_024636 | 256 |
| SLC6A14 | NM_007231 | 257 |
| GOLT1A | AB075871 | 258 |
| PKP3 | NM_007183 | 259 |
| SCEL | BC047536 | 260 |
| VTCN1 | BX648021 | 261 |
| SERPINB5 | BX640597 | 262 |
| DENND2D | AL713773 | 263 |
| PLA2G10 | NM_003561 | 264 |
| SCNN1A | AK172792 | 265 |
| GPR87 | NM_023915 | 266 |
| IRF6 | NM_006147 | 267 |
| CGN | BC146657 | 268 |
| LAMC2 | NM_005562 | 269 |
| RASGEF1B | BX648337 | 270 |
| KRTCAP3 | AY358993 | 271 |
| GRAMD2 | BC038451 | 272 |
| BSPRY | NM_017688 | 273 |
| ATP2C2 | AB014603 | 274 |
| SORBS2 | BC069025 | 275 |
| RAB25 | BE612887 | 276 |
| CLDN4 | AK126462 | 277 |
| EHF | NM_012153 | 278 |
| KRT19 | BQ073256 | 279 |
| CDS1 | NM_001263 | 280 |
| KRT16 | NM_005557 | 281 |
| CNTNAP2 | NM_014141 | 282 |
| MARVELD2 | AK055094 | 283 |
| RASEF | NM_152573 | 284 |
| INPP4B | NM_003866 | 285 |
| OVOL2 | AK022284 | 286 |
| GRHL2 | NM_024915 | 287 |
| BLNK | AK225546 | 288 |
| EPN3 | NM_017957 | 289 |
| ELF3 | NM_001114309 | 290 |
| STX19 | NM_001001850 | 291 |
| B3GNT3 | NM_014256 | 292 |
| FUT1 | NM_000148 | 293 |
| CEACAM5 | NM_004363 | 294 |
| MYO5B | NM_001080467 | 295 |
| ARHGAP8 | BC059382 | 296 |
| PRSS8 | NM_002773 | 297 |
| TTC9 | NM_015351 | 298 |
| KLK6 | NM_002774 | 299 |
| IL1RN | BC068441 | 300 |
| FAM110C | NM_001077710 | 301 |
| ALDH3B2 | AK092464 | 302 |
| PRR15 | NM_175887 | 303 |
| DSC2 | NM_004949 | 304 |
| C11orf52 | BC110872 | 305 |
| ILDR1 | BC044240 | 306 |
| CD24 | AK125531 | 307 |
| CTAGE4 | DB515636 | 308 |
| FGD2 | BC023645 | 309 |
| MYH14 | NM_001145809 | 310 |

The 60mer sequences provided in TABLES 2A and 2B are non-limiting examples of exemplary probes that correspond to a portion of the corresponding cDNA.

EMT Signature Scores were calculated for each lung cancer tumor cell line using the following method. First, a fold change differential gene expression value was calculated for each gene marker in the mesenchymal arm of the EMT Signature (see genes listed in TABLE 2A) and for each gene marker in the epithelial arm of the EMT Signature (see genes listed in TABLE 2B). This calculation was done by comparing the level of gene expression for each mesenchymal arm marker gene and epithelial arm marker gene (as measured in the lung tumor cell line microarray experiments), as compared to the level of gene expression measured for that marker gene in a human control sample, to obtain a fold change value. For the experiments depicted in FIG. 1, the human control sample values were obtained by calculating the average value for each EMT Signature gene across all 93 tumor lung cell lines. A fold-change for each EMT Signature marker gene within an individual lung tumor cell line sample was then determined with reference to the average value for that marker gene across all 93 lung tumor cell line samples. Then, a mean differential expression value for each arm of the EMT Signature (i.e., mesenchymal arm and epithelial arm), were calculated using all of the genes within each arm. Finally, the EMT Signature Score was obtained by subtracting the mean differential expression value of the epithelial arm from the mean differential expression value of the mesenchymal arm.

FIG. 1, Panel B, shows a plot of the 93 lung tumor cell lines distributed by differential CDH1 gene expression (y-axis) versus EMT signature score (x-axis). FIG. 1, Panel C, shows a plot of the 93 lung tumor cell lines distributed by EMT Signature Score (y-axis) versus VIM gene expression (x-axis).

### Example 2

### EMT Signature Score is Correlated With Response to Cancer Therapy

In this example, data are presented showing that the EMT Signature Score, described in Example 1, can be used to predict lung tumor cell response to drug treatment. Drug response experiments were performed using the same 93 lung tumor cell lines that were used to identify the EMT Signature genes, as described in Example 1 and listed in TABLES 2A and 2B. Each of the 93 lung tumor cell lines were prepared and exposed to a combination of erlotinib (N-(3-ethynylphenyl)-6,7-bis(2-methoxyethoxy)quinazolin-4-amine) (U.S. Reissue Pat. No. RE 41,065) and MK-0646 (IGF1R mAb) (U.S. Pat. No. 7,241,444; U.S. Pat. No. 7,553,485), each of which is hereby incorporated herein by reference, as described in more detail below.

### Methods:

### Cell Titration

Cells from each of the 93 lung tumor cell lines described in Example 1 were plated in DMEM supplemented with 10% fetal calf serum in 384-well tissue culture plates in 25 μL at seeding densities ranging from 500-1200 cells per well. The seeding density was chosen based on the empirically observed growth rate of the cells during expansion in flasks. A column in the plate received only medium to serve as a background control. After 24 hrs of incubation at 37 C and 5% carbon dioxide, the drug compounds erlotinib and MK-0646 were added. The drug compounds were previously titrated in a 96-well plate in DMSO at 500 times the final intended concentration and frozen at −20 C. Included in the pattern of the titration were vehicle-only control wells. On the day of the addition to the cell plates, the 500× plates containing the drug compounds were thawed. Aliquots of this plate were transferred to a 96-well plate containing the appropriate medium using automated liquid handling to create a 6× intermediate plate. Five microliters were then transferred to the cell plates to achieve the final concentration. The transfer from the 96-well format to the 384-well format was done to create quadruplicates in the 384-well plate. For each cell line, enough 384-well plates were plated and dosed to yield three time points, with triplicates at each time point.

Cell Titer Glo (Promega; Madison, Wis.) was used to assess cell mass. Cell mass was assayed at three time points: 24, 48, and 72 hours post administration of the drug compounds. Using a bulk dispenser, 25 μL per well of Cell Titer Glo was added. After two minutes of gentle mixing, the luminescence was measured from each well using an Envision plate reader (Perkin Elmer; Waltham, Mass.).

### Titration Data Analysis

The raw luminescence value for each well was corrected for background by subtracting the mean value of the luminescence from the wells on the same plate that contained no cells. For each time point there were four replicates within a plate and three replicate plates, yielding a total of 12 data points. These data points were treated equivalently and the median value was used for subsequent calculations.

For every unique combination of compound and concentration (including vehicle control) there was a set of three median values, one for each time point. A specific growth rate, μ (hr⁻¹), was regressed from this set using the equation below, where Xₜ=cell mass at time t; Xₜ₌₀=cell mass at a first time point; Δt=elapsed time (hr). Note that the specific growth rate is related to the doubling time by: μ=ln2/_{doubling}.

$$\frac{{X}_{t}}{{X}_{t = 0}} = {e}^{μΔ t} \qquad Equation 1$$

A fractional inhibition of specific growth rate corresponding to a given compound and concentration is calculated by dividing the specific growth rate at that condition, μ, by the specific growth rate in the vehicle only condition, μₘₐₓ. This ratio is a dimensionless measure of the inhibitory effect of a compound on a cell line's growth at a given concentration and is independent of the cell line's basal growth rate. However because negative specific growth rates were observed from some treatments, negative values for the ratio are obtained. The negative values make it difficult to apply many analytical techniques previously developed to handle single time point inhibition data (i.e., a ratio of treated cell mass over control cell mass at 72 hours). A transformation is applied to the μ/μₘₐₓ ratio to convert it to fixed time point-like data while still maintaining its independence from variation in basal growth rates. Equation 1 was applied to a treatment condition and to a control condition, the ratio was taken, and after rearrangement, the equation below results, where X=cell mass in treatment condition at time t; X₀=cell mass in control condition at time t.

$$\frac{X}{{X}_{0}} = {e}^{( \frac{μ}{{μ}_{max}} 1 ) {μ}_{max} t} \qquad Equation 2$$

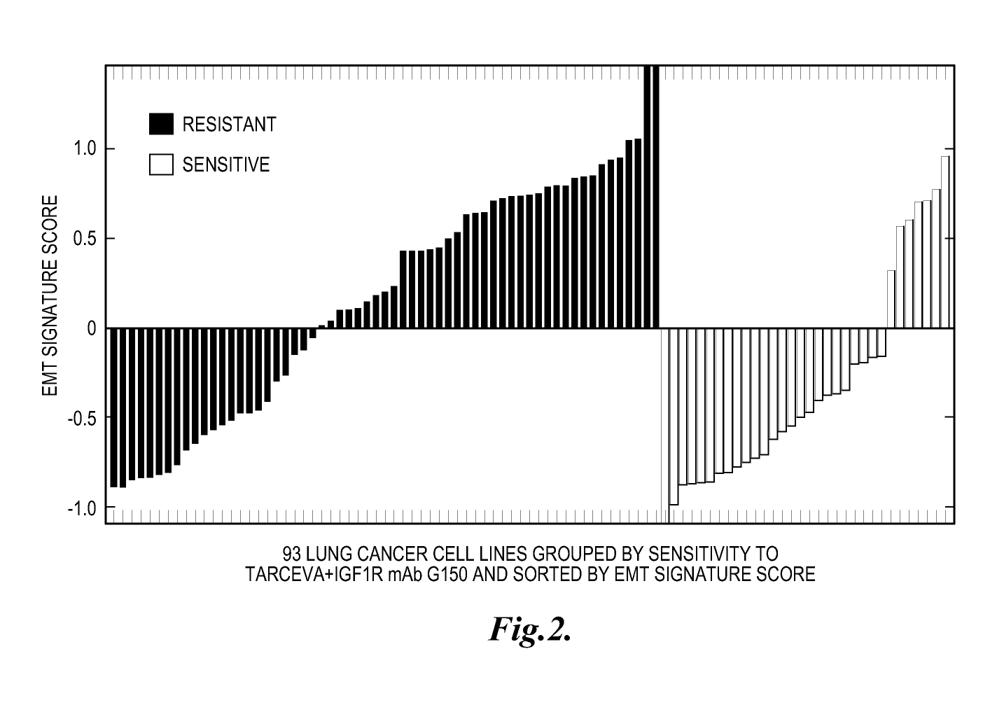

Equation 2 describes a fixed time point type of inhibition (X/X₀) as a function of the μ/μₘₐₓ ratio and also the dimensionless term μₘₐₓ t. The value of e to the power of μₘₐₓ t is the fold change observed in the control treatment. In the traditional experiment, t is fixed (at 72 hours for example) and the fold change is a function of μₘₐₓ. However, when comparing data across cell lines, varying basal growth rates will cause the fold changes at a fixed time point to also vary. It is proposed that a superior method is to compare cell lines' responses at a fixed fold change, removing the effect of the variation in basal growth rates. This is accomplished mathematically by fixing the value of the term μₘₐₓ t in Equation 2 to a constant. For the data presented in TABLE 5 and FIG. 2, the value of 1.4 was chosen, as this corresponds to 4-fold growth, a value that was realized in many of the cell lines during the 72 hour experimental duration. Thus, Equation 2 becomes:

$$\frac{X}{{X}_{0}} = {e}^{1.4 ( \frac{μ}{{μ}_{max}} - 1 )} \qquad Equation 3$$

The values of X/X₀ were used as the metric of response in the lung tumor cell line panel of 93 cell lines.

### Evaluation of Cell Lines' Reponses

In order to stratify the cell lines' responses to the drug compounds, a single metric of response is desired. The customary approach is to use the concentration required to produce a certain fractional effect (i.e., IC₅₀, GI₅₀, etc). However, in this lung tumor cell line panel the drug compounds produced titration curve shapes that made this approach less suitable. Many cell lines showed incomplete inhibition even at very high doses. Also, the sigmoidicity of the curves varied amongst the cell lines in response to the same drug compound. In fact, many investigators have suggested that the sigmoidicity of cell lines' responses is more likely due to heterogeneity of the cell population rather than to the kinetics of the inhibitor (Hassan et al., J. Pharmacol Exp. Ther. 299:1140-1147). Since the sigmoidicity of the dose-response curves can significantly impact IC₅₀-type values, a different metric is preferred.

Instead of fixing a fractional effect and evaluating concentrations required to produce it, one can pick a concentration at which to evaluate response across the cell lines. The choice of concentration is important. Some suggest using predetermined biochemical IC₅₀'s to guide the choice. Here a strategy is presented for determining the optimal concentration at which to evaluate a response that uses only the data collected in the experiment.

Given that stratification of the cell lines' relative responses is paramount, the metric should maximize the power to discriminate between individual cell line's responses. Our approach was to use a computational algorithm to find the concentration at which the population of cell lines' responses exhibited maximal variation. This was done by finding the maximum value of the variance across the concentration range tested. Using this concentration of maximal variation, X/Xₒ was evaluated for each cell line. This value is referred to as the Inhibition at Maximum Variance (IMV).

### Drug Treatment

Tarceva was obtained from Lc Laboratories (as Erlotinib Powder HCl Salt); IGF1R mAB was obtained from Merck (MK-0646). The 93 cell lines were treated by either Tarceva alone, MK-0646 alone, and the combination of Tarceva and MK-0646. Tarceva was titrated at 8 concentrations ranging from 4 nM to 10 μM. IGF1R mAb (MK-0646) was titrated at 8 concentrations ranging from 0.4 μg/mL to 100 μg/mL. For the combination, the concentration of MK-0646 was fixed at 10 μg/mL while Tarceva was titrated at 8 concentrations ranging from 4 nM to 10 μM. Growth rates of the cell lines were measured either in the presence of the drug treatments, or absence of drug (DMSO control). The growth rate under DMSO treatment was used as a control to derive the relative growth rates for the cell lines under treatments.

### Results

FIG. 2 shows a waterfall plot of 93 lung cancer cell lines classified as being resistant or sensitive to cell growth inhibition by exposure to erlotinib (Tarceva) plus IGF1R mAb G150 (MK-0646) and sorted by EMT Signature score (Accuracy=0.68, Sensitivity=0.78, Specificity=0.62, Fisher Extract Test p-value=2e-4, ROC AUC=1−0.71).

TABLE 3 shows the EMT Signature score and Inhibition at Maximum Variance (IMV) value for each of the 93 lung tumor cell lines. Tumor cell lines having an IMV of 0.50 or higher were classified as being resistant to growth inhibition after treatment with the combination of Tarceva and MK-0646.

TABLE 3
List of 93 Lung Tumor Cell Lines Showing EMT Signature
Score and Sensitivity (IMV) to Exposure to Erlotinib
(Tarceva) + IGF1R mAB (MK-0646)

| Lung Tumor | EMT | EMT | IMV |
|---|---|---|---|
| Cell Line | Classification | Signature | Tarceva + |
| Name | Group | Score | MK-0646 |
| HLFa | Mesenchymal | 1.34 | 0.53 |
| Hs573.T | Mesenchymal | 1.34 | 0.96 |
| MSTO-211H | Mesenchymal | 0.95 | 0.91 |
| H2052 | Mesenchymal | 0.93 | 0.75 |
| H2122 | Mesenchymal | 0.86 | 0.08 |
| H2452 | Mesenchymal | 0.85 | 0.82 |
| CALU-1 | Mesenchymal | 0.84 | 1.00 |
| H1792 | Mesenchymal | 0.78 | 0.58 |
| LU99A | Mesenchymal | 0.74 | 0.53 |
| LXF289 | Mesenchymal | 0.72 | 0.73 |
| H1299 | Mesenchymal | 0.72 | 0.84 |
| H1563 | Mesenchymal | 0.71 | 1.00 |
| H661 | Mesenchymal | 0.70 | 0.67 |
| H1703 | Mesenchymal | 0.70 | 0.99 |
| LCLC103H | Mesenchymal | 0.67 | 0.82 |
| H1915 | Mesenchymal | 0.67 | 0.92 |
| SW1573 | Mesenchymal | 0.66 | 0.63 |
| H460 | Mesenchymal | 0.66 | 0.80 |
| SKMES1 | Mesenchymal | 0.65 | 0.17 |
| COLO-699N | Mesenchymal | 0.63 | 0.40 |
| H226 | Mesenchymal | 0.63 | 0.94 |
| H2172 | Mesenchymal | 0.60 | 0.80 |
| COLO699 | Mesenchymal | 0.59 | 0.48 |
| RERF_LC_MS | Mesenchymal | 0.58 | 0.69 |
| H2030 | Mesenchymal | 0.58 | 0.48 |
| H23 | Mesenchymal | 0.57 | 0.67 |
| H28 | Mesenchymal | 0.54 | 0.39 |
| H522 | Mesenchymal | 0.49 | 0.69 |
| A549 | Mesenchymal | 0.46 | 0.77 |
| HCC44 | Mesenchymal | 0.42 | 0.68 |
| H647 | Mesenchymal | 0.41 | 0.75 |
| H1755 | Mesenchymal | 0.39 | 0.73 |
| A427 | Mesenchymal | 0.39 | 0.71 |
| H1793 | Mesenchymal | 0.21 | 0.85 |
| H2023 | Mesenchymal | 0.18 | 0.89 |
| HCC15 | Mesenchymal | 0.16 | 0.65 |
| H2228 | Mesenchymal | 0.12 | 0.51 |
| H596 | Mesenchymal | 0.10 | 0.58 |
| H2073 | Mesenchymal | −0.15 | 0.33 |
| H1650 | Epithelial | −0.13 | 0.62 |
| H1944 | Epithelial | −0.14 | 0.32 |
| H1693 | Epithelial | −0.15 | 0.26 |
| CORL_105 | Epithelial | −0.16 | 0.11 |
| HARA | Epithelial | −0.33 | 0.48 |
| H1838 | Epithelial | −0.34 | 0.45 |
| HARA_B | Epithelial | −0.34 | 0.41 |
| H1734 | Epithelial | −0.35 | 0.24 |
| H1568 | Epithelial | −0.43 | 0.16 |
| RERF_LC_ad2 | Epithelial | −0.43 | 0.93 |
| UMC-11 | Epithelial | −0.44 | 0.56 |
| H292 | Epithelial | −0.45 | 0.39 |
| CHAGO-K-1 | Epithelial | −0.46 | 0.61 |
| COLO_668 | Epithelial | −0.50 | 0.69 |
| CAL12T | Epithelial | −0.51 | 0.38 |
| KNS62 | Epithelial | −0.59 | 0.99 |
| H1993 | Epithelial | −0.60 | 0.65 |
| H1666 | Epithelial | −0.64 | 0.34 |
| H727 | Epithelial | −0.65 | 0.42 |
| CORL23/R | Epithelial | −0.71 | 0.70 |
| HCC827 | Epithelial | −0.73 | 0.09 |
| LUDLU1 | Epithelial | −0.73 | 0.05 |
| HCC78 | Epithelial | −0.75 | 1.00 |
| H1573 | Epithelial | −0.75 | 0.64 |
| CORL-23/CPR | Epithelial | −0.75 | 0.73 |
| H1648 | Epithelial | −0.75 | 0.54 |
| H2342 | Epithelial | −0.78 | 0.73 |
| H2170 | Epithelial | −0.79 | 0.31 |
| CORL23 | Epithelial | −0.80 | 0.46 |
| DV90 | Epithelial | −0.80 | 0.34 |
| H1437 | Epithelial | −0.81 | 0.55 |
| H1869 | Epithelial | −0.81 | 0.21 |
| CORL23/R23- | Epithelial | −0.83 | 0.82 |
| H441 | Epithelial | −0.88 | 0.47 |
| H2126 | Epithelial | −1.00 | 0.29 |
| SKLU1 | Intermediate | 0.82 | 0.59 |
| H1155 | Intermediate | 0.38 | 0.90 |
| H1651 | Intermediate | 0.28 | 0.48 |
| HCC 366 | Intermediate | 0.17 | 0.08 |
| H2085 | Intermediate | 0.08 | 0.67 |
| H520 | Intermediate | 0.04 | 1.00 |
| H2106 | Intermediate | 0.01 | 1.00 |
| LK2 | Intermediate | −0.04 | 0.61 |
| H2444 | Intermediate | −0.12 | 0.55 |
| PC7 | Intermediate | −0.21 | 0.81 |
| EPLC_272H | Intermediate | −0.25 | 0.50 |
| H2009 | Intermediate | −0.39 | 0.64 |
| H1975 | Intermediate | −0.42 | 0.94 |
| HCC4006 | Intermediate | −0.48 | 0.00 |
| EBC1 | Intermediate | −0.51 | 0.82 |
| H2347 | Intermediate | −0.52 | 1.00 |
| H1395 | Intermediate | −0.52 | 0.49 |
| CALU3 | Intermediate | −0.70 | 0.12 |
| H358 | Intermediate | −0.73 | 0.16 |

The data in this Example show that the EMT Signature score significantly correlates with lung tumor cell line resistance to growth inhibition after combination treatment with erlotinib-MK-0646 with high specificity. In particular, lung cancer cell lines that have a high EMT signature score are predominantly resistant to treatment (i.e., exposure to the combination of compounds does not significantly inhibit cell growth).

Therefore, the results in this Example demonstrate that the EMT Signature score of a cell is useful as a predictor of the sensitivity of the cell to treatment with a therapeutic agent.

### Example 3

### Identification of a First Principal Component Gene Set (PC1) in Colon Cancer Tumor Samples That is Correlated to the EMT Signature

Colon cancer has been classically described by clinicopathologic features that permit the prediction of outcome only after surgical resection and staging. To better characterize the disease, an unsupervised analysis of microarray data from 326 colon cancers from a spectrum of clinical stages was performed to identify the first principal component (PC1) of the most variable set of differentially expressed genes.

### Methods:

326 human colorectal cancer (“CRC”) samples derived from the Moffitt Cancer Center, were previously assessed using a single Affymetrix U133Plus2.0 platform and single standard operating procedure at described in Jorissen R. N. et al., Clin Cancer Res 15(24):7642-51 (2009), incorporated herein by reference; and the Gene Expression Omnibus (GEO) Series GSE14333, at ncbi.nlm.nih.gov/geo/query/acc.cgi?acc=GSE14333.

Formalin fixed paraffin blocks (FFPE) were obtained for 69 of these cases and used to extract tumor RNA after macrodissection. The microarray data was processed by running the RNA normalization method as implemented in Affy Power Tools using default settings, background correction and quantile normalization with subsequent application of log 10 to obtained probe intensities.

Unsupervised analysis of the most variable genes expressed in the CRC data set (n=326) was undertaken to discover new, “intrinsic” biology of colon cancer. Principal component analysis on the entire gene expression data set of 326 CRC samples, as implemented in the Princomp function in Mathlab, Mathworks Inc., was computed by selecting the 1st principal component (PC1) corresponding to the highest eigenvalue of the covariance matrix, describing the inherent variability of the data.

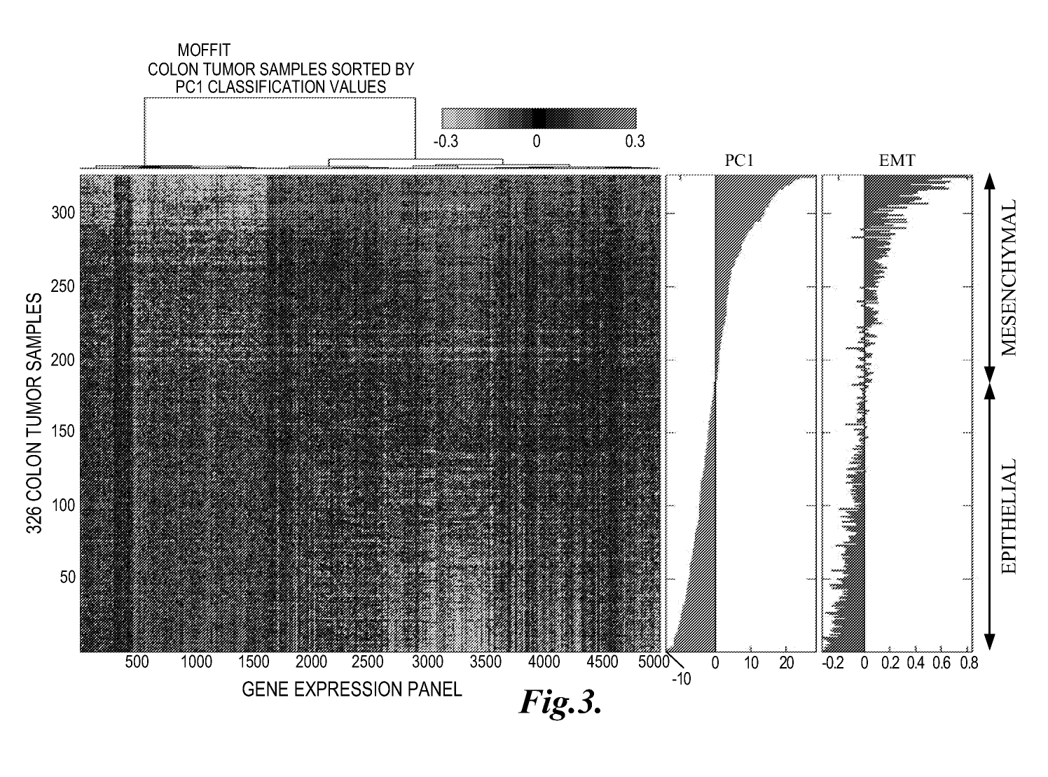

The first principal component identified from these analyses of the CRC samples contained about 5,000 differentially expressed genes. The PC1 genes allowed classification of the 326 CRC tumor samples into two major subpopulations based on gene expression values. FIG. 3 visually illustrates the intrinsic molecular stratification of the 326 human CRC samples in the Moffitt sample set with respect to the gene expression level for the panel of 5,000 PC1 genes. Unsupervised analysis and hierarchical clustering of global gene expression data derived from the Moffitt CRC cases identified two major “intrinsic” subclasses distinguished by the first principal component (PC1) of the most variable genes.

The subpanels on the far right of FIG. 3 show that the PC1 Signature score for each colorectal cancer sample is tightly correlated with the EMT Signature score calculated for each sample as described in Example 1, above. The PC1 Signature Score was calculated for each of the Moffitt CRC samples by the same method as described above for the EMT Signature score. The PC1 Signature genes clearly distinguish two subclasses which correspond to the epithelial cell-like and mesenchymal cell-like classifications obtained using the EMT Signature Score.

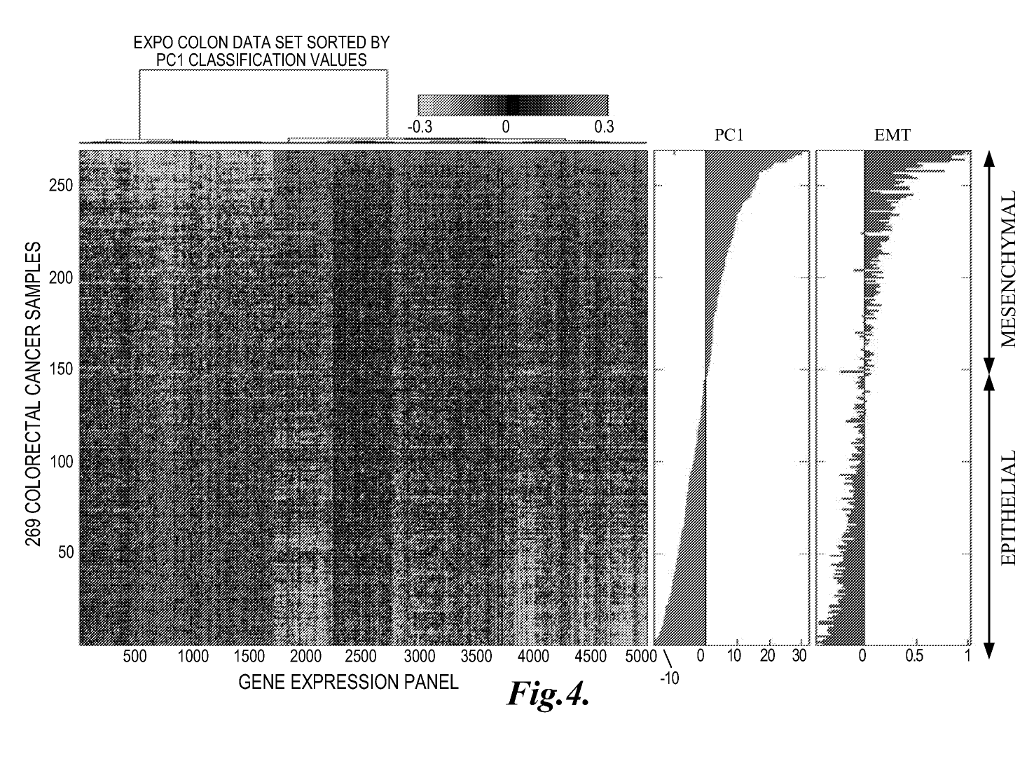

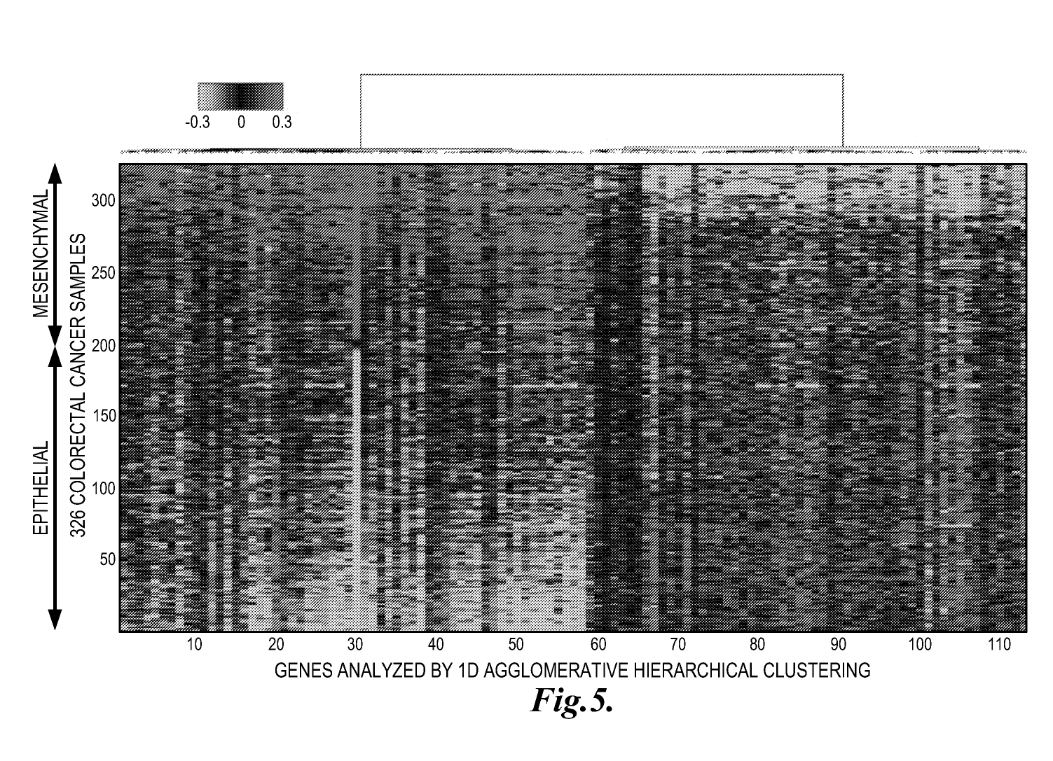

The classification power of the PC1 Signature scores and EMT Signature scores were confirmed in an independent ExPO data set (n=269) (FIG. 4) derived from an independent set of human CRC samples, suggesting that the EMT Signature genes are part of a pervasive program underpinning colon cancer biology. FIG. 4 visually illustrates the intrinsic molecular stratification of the 326 human CRC samples in the ExPO data set with respect to the gene expression level for the panel of 5,000 PC1 genes. The ExPO data set is publicly accessible at Expression Project of Oncology (ExPO), Series GSE2109, at ncbi.nlm.nih.gov/projects/geo/query/acc.cgi?acc=GSE2109.

### Example 4

### Selection of a PC1 Signature

A refined set of PC1 Signature genes were selected from the about 5000 PC1 genes identified in Example 3, above, by performing Principal Component Analysis (“PCA”) on robust multi-array (RMA)-normalized data obtained from the U133 Plus 2.0 Affymetrix arrays. The RMA-normalized dataset consisted of the 326 CRC tumor profiles described in Example 3. A first principal component was selected and for each probe-set, (i.e., gene transcript represented on the array), a Spearman correlation was computed to the PC1. Then, the 200 probe-sets with the highest value of correlation coefficient to PC1 were selected, and the list of unique markers for these probe-sets was used to generate the 124 PC1 Signature Mesenchymal marker list shown in TABLE 4A. TABLE 4A provides for each of the 124 PC1 Signature Mesenchymal markers, the gene symbol; the Genbank reference number for each gene symbol as of Oct. 1, 2010, each of which is hereby incorporated herein by reference; and the SEQ ID NO: corresponding to an exemplary 60-mer sequence that corresponds to a portion of the corresponding cDNA, which may be used as a probe.

TABLE 4A
124 PC1 Signature Genes: The Mesenchymal or Up-Regulated Arm.

| | Gene Transcript | |
|---|---|---|
| | Genbank | Transcript |
| | Reference | probe SEQ |
| Gene Symbol | Number | ID NO: |
| SPARC | AK126525 | 7 |
| CAP2 | NM_006366 | 13 |
| JAM3 | AK027435 | 18 |
| SRPX | BC020684 | 19 |
| NAP1L3 | BC094729 | 30 |
| CMTM3 | AK056324 | 38 |
| MAP1B | NM_005909 | 43 |
| MSRB3 | NM_001031679 | 45 |
| AKAP12 | NM_005100 | 52 |
| RECK | BX648668 | 59 |
| ZFPM2 | NM_012082 | 67 |
| ATP8B2 | NM_020452 | 69 |
| LGALS1 | BF570935 | 76 |
| HTRA1 | NM_002775 | 94 |
| NDN | NM_002487 | 95 |
| LHFP | NM_005780 | 97 |
| PRKD1 | X75756 | 98 |
| UCHL1 | AB209038 | 100 |
| DPYSL3 | BC077077 | 101 |
| DFNA5 | AK094714 | 103 |
| MRAS | NM_012219 | 104 |
| FLRT2 | NM_013231 | 106 |
| VIM | NM_003380 | 122 |
| LIX1L | AK128733 | 123 |
| AP1S2 | BX647483 | 127 |
| GFPT2 | BC000012 | 134 |
| TRPA1 | Y10601 | 135 |
| GNG11 | BF971151 | 139 |
| ARMCX1 | CR933662 | 142 |
| PTRF | NM_012232 | 148 |
| AEBP1 | NM_001129 | 311 |
| AKT3 | NM_005465 | 312 |
| AMOTL1 | NM_130847 | 313 |
| ANKRD6 | NM_014942 | 314 |
| ARMCX2 | NM_014782 | 315 |
| BASP1 | NM_006317 | 316 |
| BGN | NM_001711 | 317 |
| C1orf54 | NM_024579 | 318 |
| C20orf194 | NM_001009984 | 319 |
| CALD1 | NM_004342 | 320 |
| CCDC80 | NM_199511 | 321 |
| CEP170 | NM_001042404 | 322 |
| CFH | NM_000186 | 323 |
| CFL2 | NM_021914 | 324 |
| COX7A1 | NM_001864 | 325 |
| CRYAB | NM_001885 | 326 |
| DCN | NM_001920 | 327 |
| DNAJB4 | NM_007034 | 328 |
| DZIP1 | NM_014934 | 329 |
| ECM2 | NM_001393 | 330 |
| EFHA2 | NM_181723 | 331 |
| EFS | NM_005864 | 332 |
| EHD3 | NM_014600 | 333 |
| FAM20C | NM_020223 | 334 |
| FBXL7 | NM_012304 | 335 |
| FEZ1 | NM_005103 | 336 |
| FRMD6 | NM_001042481 | 337 |
| GLIS2 | NM_032575 | 338 |
| HECTD2 | NM_173497 | 339 |
| IL1R1 | NM_000877 | 340 |
| KCNE4 | NM_080671 | 341 |
| KIAA1462 | NM_020848 | 342 |
| KLHL5 | NM_001007075 | 343 |
| LAYN | NM_178834 | 344 |
| LDB2 | NM_001130834 | 345 |
| LMCD1 | NM_014583 | 346 |
| LPHN2 | NM_012302 | 347 |
| LZTS1 | NM_021020 | 348 |
| MAF | NM_001031804 | 349 |
| MAGEH1 | NM_014061 | 350 |
| MAP9 | NM_001039580 | 351 |
| MCC | NM_001085377 | 352 |
| MGP | NM_000900 | 353 |
| MLLT11 | NM_006818 | 354 |
| MPDZ | NM_003829 | 355 |
| MSN | NM_002444 | 356 |
| MXRA7 | NM_001008528 | 357 |
| MYH10 | NM_005964 | 358 |
| MYO5A | NM_000259 | 359 |
| NNMT | NM_006169 | 360 |
| NR3C1 | NM_000176 | 361 |
| NRP1 | NM_001024628 | 362 |
| NRP2 | NM_003872 | 363 |
| PEA15 | NM_003768 | 364 |
| PFTK1 | NM_012395 | 365 |
| PHLDB2 | NM_001134437 | 366 |
| PKD2 | NM_000297 | 367 |
| PRICKLE1 | NM_001144881 | 368 |
| PTPRM | NM_001105244 | 369 |
| QKI | NM_006775 | 370 |
| RAB31 | NM_006868 | 371 |
| RAB34 | NM_001142624 | 372 |
| RAI14 | NM_001145520 | 373 |
| RASSF8 | NM_001164746 | 374 |
| RGS4 | NM_001102445 | 375 |
| RNF180 | NM_001113561 | 376 |
| SCHIP1 | NM_014575 | 377 |
| SDC2 | NM_002998 | 378 |
| SERPINF1 | NM_002615 | 379 |
| SGCE | NM_001099400 | 380 |
| SGTB | NM_019072 | 381 |
| SLIT2 | NM_004787 | 382 |
| SMARCA1 | NM_003069 | 383 |
| SNAI2 | NM_003068 | 384 |
| SPG20 | NM_001142294 | 385 |
| SRGAP2 | NM_001042758 | 386 |
| STON1 | NM_006873 | 387 |
| SYT11 | NM_152280 | 388 |
| TCEA2 | NM_003195 | 389 |
| TCEAL3 | NM_001006933 | 390 |
| TIMP2 | NM_003255 | 391 |
| TNS1 | NM_022648 | 392 |
| TPST1 | NM_003596 | 393 |
| TRPC1 | NM_003304 | 394 |
| TRPS1 | NM_014112 | 395 |
| TSPYL5 | NM_033512 | 396 |
| TTC7B | NM_001010854 | 397 |
| TUBB6 | NM_032525 | 398 |
| TUSC3 | NM_006765 | 399 |
| UBE2E2 | NM_152653 | 400 |
| WWTR1 | NM_001168278 | 401 |
| ZNF25 | NM_145011 | 402 |
| ZNF532 | NM_018181 | 403 |
| ZNF677 | NM_182609 | 404 |

Similarly, 200 probe-sets with the most negative correlation coefficient to PC1 were taken, and the corresponding list of 119 unique markers was used to generate the PC1 Signature Epithelial marker list shown in TABLE 4B. TABLE 4B provides for each of the 119 PC1 Signature Epithelial markers, the gene symbol; the Genbank reference number for each gene symbol as of Oct. 1, 2010, each of which is hereby incorporated herein by reference; and the SEQ ID NO: corresponding to an exemplary 60-mer sequence that corresponds to a portion of the corresponding cDNA, which may be used as a probe.

TABLE 4B
119 PC1 Signature Genes: The Epithelial or Down-Regulated Arm.

| | Gene Transcript | Transcript |
|---|---|---|
| | Genbank | probe |
| Gene | Reference | SEQ ID |
| Symbol | Number | NO: |
| TMC5 | NM_001105248 | 160 |
| FUT3 | NM_000149 | 173 |
| AP1M2 | BC005021 | 177 |
| FAM84A | NM_145175 | 190 |
| GPX2 | BE512691 | 192 |
| CKMT1B | AK094322 | 213 |
| FA2H | NM_024306 | 223 |
| MAP7 | NM_003980 | 230 |
| ST14 | NM_021978 | 241 |
| MARVELD3 | NM_001017967 | 248 |
| RAB25 | BE612887 | 276 |
| CDS1 | NM_001263 | 280 |
| EPN3 | NM_017957 | 289 |
| MYO5B | NM_001080467 | 295 |
| MYH14 | NM_001145809 | 310 |
| ACOT11 | NM_015547 | 405 |
| AGMAT | NM_024758 | 406 |
| ANKS4B | NM_145865 | 407 |
| ATP10B | NM_025153 | 408 |
| AXIN2 | NM_004655 | 409 |
| BCAR3 | NM_003567 | 410 |
| BCL2L14 | NM_030766 | 411 |
| BDH1 | NM_004051 | 412 |
| BRI3BP | NM_080626 | 413 |
| C10orf99 | NM_207373 | 414 |
| C4orf19 | NM_001104629 | 415 |
| C9orf152 | NM_001012993 | 416 |
| C9orf75 | NM_001128228 | 417 |
| C9orf82 | NM_001167575 | 418 |
| CALML4 | NM_001031733 | 419 |
| CAPN5 | NM_004055 | 420 |
| CASP5 | NM_001136109 | 421 |
| CASP6 | NM_001226 | 422 |
| CBLC | NM_001130852 | 423 |
| CC2D1A | NM_017721 | 424 |
| CCL28 | NM_148672 | 425 |
| CDC42EP5 | NM_145057 | 426 |
| CDX1 | NM_001804 | 427 |
| CLDN3 | NM_001306 | 428 |
| CMTM4 | NM_178818 | 429 |
| CORO2A | NM_003389 | 430 |
| COX10 | NM_001303 | 431 |
| CYP2J2 | NM_000775 | 432 |
| DAZAP2 | NM_001136264 | 433 |
| DDAH1 | NM_001134445 | 434 |
| DTX2 | NM_001102594 | 435 |
| DUOX2 | NM_014080 | 436 |
| DUOXA2 | NM_207581 | 437 |
| ENTPD5 | NM_001249 | 438 |
| EPB41L4B | NM_018424 | 439 |
| EPHB2 | NM_004442 | 440 |
| EPS8L3 | NM_024526 | 441 |
| ESRRA | NM_004451 | 442 |
| ETHE1 | NM_014297 | 443 |
| EXPH5 | NM_001144763 | 444 |
| F2RL1 | NM_005242 | 445 |
| FAM3D | NM_138805 | 446 |
| FAM83F | NM_138435 | 447 |
| FRAT2 | NM_012083 | 448 |
| FUT2 | NM_000511 | 449 |
| FUT4 | NM_002033 | 450 |
| FUT6 | NM_000150 | 451 |
| GALNT7 | NM_017423 | 452 |
| GMDS | NM_001500 | 453 |
| GPA33 | NM_005814 | 454 |
| GPR35 | NM_005301 | 455 |
| HDHD3 | NM_031219 | 456 |
| HMGA1 | NM_002131 | 457 |
| HNF4A | NM_000457 | 458 |
| HOXB9 | NM_024017 | 459 |
| HSD11B2 | NM_000196 | 460 |
| KALRN | NM_001024660 | 461 |
| KCNE3 | NM_005472 | 462 |
| KCNQ1 | NM_000218 | 463 |
| KIAA0152 | NM_014730 | 464 |
| LENG9 | NM_198988 | 465 |
| LGALS4 | NM_006149 | 466 |
| LRRC31 | NM_024727 | 467 |
| MCCC2 | NM_022132 | 468 |
| MPST | NM_001013436 | 469 |
| MRPS35 | NM_021821 | 470 |
| MUC3B | XM_001125753.2 | 471 |
| MYB | NM_001130172 | 472 |
| MYO7B | NM_001080527 | 473 |
| NAT2 | NM_000015 | 474 |
| NOB1 | NM_014062 | 475 |
| NOX1 | NM_007052 | 476 |
| NR1I2 | NM_003889 | 477 |
| PAQR8 | NM_133367 | 478 |
| PI4K2B | NM_018323 | 479 |
| PKP2 | NM_001005242 | 480 |
| PLA2G12A | NM_030821 | 481 |
| PLEKHA6 | NM_014935 | 482 |
| PLS1 | NM_001145319 | 483 |
| PMM2 | NM_000303 | 484 |
| POF1B | NM_024921 | 485 |
| PPP1R1B | NM_032192 | 486 |
| PREP | NM_002726 | 487 |
| RNF186 | NM_019062 | 488 |
| SELENBP1 | NM_003944 | 489 |
| SH3RF2 | NM_152550 | 490 |
| SHH | NM_000193 | 491 |
| SLC12A2 | NM_001046 | 492 |
| SLC27A2 | NM_001159629 | 493 |
| SLC29A2 | NM_001532 | 494 |
| SLC35A3 | NM_012243 | 495 |
| SLC37A1 | NM_018964 | 496 |
| SLC44A4 | NM_001178044 | 497 |
| SLC5A1 | NM_000343 | 498 |
| SLC9A2 | NM_003048 | 499 |
| STRBP | NM_001171137 | 500 |
| SUCLG2 | NM_001177599 | 501 |
| SULT1B1 | NM_014465 | 502 |
| TJP3 | NM_014428 | 503 |
| TMEM54 | NM_033504 | 504 |
| TMPRSS2 | NM_001135099 | 505 |
| TST | NM_003312 | 506 |
| USP54 | NM_152586 | 507 |
| XK | NM_021083 | 508 |

The markers represented in TABLES 4A and 4B are collectively referred to as the PC1 Signature. Markers that are also present in the EMT Signature lists (Example 1, TABLES 2A and 2B), are indicated at the beginning of both TABLES 4A and 4B. In total, 30 gene markers listed in TABLE 4A are also present in TABLE 2A, and 15 gene markers listed in TABLE 4B are also present in TABLE 2B. The 60mer sequences provided in TABLES 4A and 4B are non-limiting examples of exemplary probes that correspond to a portion of the corresponding cDNA.

### Example 5

### Association of the PC1 and EMT Signatures with Epithelial-to-Mesenchymal Biological Processes

To further clarify the association of the EMT biological pathway with the PC1 Signature and EMT Signature, the 326 Moffitt colorectal cancer tumor samples used to generate the PC1 signature, sorted by PC1, were analyzed in a hierarchical cluster analysis of the top 100 individual genes assessed from a text mining approach which involved literature searching for genes shown to be upregulated in epithelial or mesenchymal cells, along with representative signatures of genes, shown in TABLE 5 below.

The set of 100 individual genes shown below in TABLE 5 includes CDH1, CLDN9, FGFR1, TWIST1&2, AXL, VIM, as well as gene signatures (PC1, EMT, TGFbeta, Proliferation, MYC, and RAS).

TABLE 5
Individual Genes and Signatures of Genes analyzed in FIG. 5.

| Reference | | | |
|---|---|---|---|
| number | | Type: | Upregulated in |
| with regard to | Gene | individual | Mesenchymal (M) |
| FIG. 5 | or Gene | gene or gene | or Epithelial (E) |
| (horizontal) | signature | signature | (in FIG. 5) |
| 1 | TGFBR1 | Individual | M |
| 2 | ACVR1 | Individual | M |
| 3 | RNF11 | Individual | M |
| 4 | NFIC | Individual | M |
| 5 | ETV5 | Individual | M |
| 6 | SLC39A6 | Individual | M |
| 7 | SMAD3 | Individual | M |
| 8 | FOXC1 | Individual | M |
| 9 | FOXC2 | Individual | M |
| 10 | CDON | Individual | M |
| 11 | GLI3 | Individual | M |
| 12 | CDH2 | Individual | M |
| 13 | FGF1 | Individual | M |
| 14 | TIAM1 | Individual | M |
| 15 | SMAD1 | Individual | M |
| 16 | FN1 | Individual | M |
| 17 | FGF7 | Individual | M |
| 18 | GLIS2 | Individual | M |
| 19 | FBLN1 | Individual | M |
| 20 | MEOX2 | Individual | M |
| 21 | GLI2 | Individual | M |
| 22 | LAMB2 | Individual | M |
| 23 | MAP3K3 | Individual | M |
| 24 | TCF4 | Individual | M |
| 25 | FGFR1 | Individual | M |
| 26 | DZIP1 | Individual | M |
| 27 | FLRT2 | Individual | M |
| 28 | RECK | Individual | M |
| 29 | SRPX | Individual | M |
| 30 | PC1 | Signature | M |
| 31 | EMT | Signature | M |
| 32 | ARMCX1 | Individual | M |
| 33 | VEGFB | Individual | M |
| 34 | WASF3 | Individual | M |
| 35 | STX2 | Individual | M |
| 36 | SFRP1 | Individual | M |
| 37 | FBLN5 | Individual | M |
| 38 | EPHA3 | Individual | M |
| 39 | SH2D3C | Individual | M |
| 40 | MMRN2 | Individual | M |
| 41 | MRAS | Individual | M |
| 42 | WISP1 | Individual | M |
| 43 | MSN | Individual | M |
| 44 | VIM | Individual | M |
| 45 | SNAI2 | Individual | M |
| 46 | TWIST2 | Individual | M |
| 47 | TGFbeta | Signature | M |
| 48 | TWIST1 | Individual | M |
| 49 | AXL | Individual | M |
| 50 | TAGLN | Individual | M |
| 51 | TGFB1I1 | Individual | M |
| 52 | HTRA1 | Individual | M |
| 53 | SPARC | Individual | M |
| 54 | ASPN | Individual | M |
| 55 | CTGF | Individual | M |
| 56 | MGP | Individual | M |
| 57 | ECM2 | Individual | M |
| 58 | ZFPM2 | Individual | M |
| 59 | SIP1 | Individual | M |
| 60 | PROLIFERATION | Signature | E |
| 61 | MYC | Signature | E |
| 62 | RSL1D1 | Individual | E |
| 63 | KAZALD1 | Individual | E |
| 64 | LYPD5 | Individual | E |
| 65 | CLDN9 | Individual | E |
| 66 | CD44 | Individual | E |
| 67 | LCN2 | Individual | E |
| 68 | CRB3 | Individual | E |
| 69 | MET | Individual | E |
| 70 | RAS | Signature | E |
| 71 | S100P | Individual | E |
| 72 | TNS4 | Individual | E |
| 73 | CLDN7 | Individual | E |
| 74 | KRT18 | Individual | E |
| 75 | KRT8 | Individual | E |
| 76 | RBM35A | Individual | E |
| 77 | SOX9 | Individual | E |
| 78 | MAL2 | Individual | E |
| 79 | CDH1 | Individual | E |
| 80 | CLDN4 | Individual | E |
| 81 | ELF3 | Individual | E |
| 82 | OCLN | Individual | E |
| 83 | CCL14 | Individual | E |
| 84 | CEACAM1 | Individual | E |
| 85 | EVI1 | Individual | E |
| 86 | CD24 | Individual | E |
| 87 | PRSS8 | Individual | E |
| 88 | TMPRSS4 | Individual | E |
| 89 | MMP15 | Individual | E |
| 90 | RBM35B | Individual | E |
| 91 | DSC2 | Individual | E |
| 92 | ITGB4 | Individual | E |
| 93 | MST1R | Individual | E |
| 94 | JUP | Individual | E |
| 95 | SPINT1 | Individual | E |
| 96 | SDC1 | Individual | E |
| 97 | PKP3 | Individual | E |
| 98 | KRT19 | Individual | E |
| 99 | SFN | Individual | E |
| 100 | FOXD2 | Individual | E |
| 101 | AREG | Individual | E |
| 102 | GSK3B | Individual | E |
| 103 | ISX | Individual | E |
| 104 | ETS2 | Individual | E |
| 105 | TDGF1 | Individual | E |
| 106 | CDX2 | Individual | E |
| 107 | CDX1 | Individual | E |
| 108 | IHH | Individual | E |
| 109 | SHH | Individual | E |
| 110 | FOXA2 | Individual | E |
| 111 | BCAR3 | Individual | E |
| 112 | KIAA0152 | Individual | E |
| 113 | EPHB3 | Individual | E |

As shown in FIG. 5, the hierarchical cluster analysis of the top 100 genes, assessed from a text mining approach, were strongly associated with the Epithelial-to-Mesenchymal transition (EMT) program, as shown on the 326 Moffitt Colorectal cancer tumor samples sorted by PC1 score. In FIG. 5, the genes/gene signatures up-regulated in mesenchymal tumors are shown in magenta (darker greyscale), and the genes/gene signatures that are up-regulated in epithelial tumors are shown in cyan (lighter greyscale). These results shown in FIG. 5 are summarized above in TABLE 5.

The 100 genes shown in TABLE 5 that were analyzed in FIG. 5 include genes previously linked to the EMT program such as VIM, FGFR, FLT1, FN1, TWIST1, TWIST2, AXL, and TCF, were individually assessed and found to be positively correlated with PC1 Signature and EMT Signature Scores (FIG. 5). Similarly, genes such as CDH1, CLDN9, EGFR, and MET were negatively correlated with PC1 Signature and EMT Signature Scores (FIG. 5). As shown above in TABLE 5 and FIG. 5, the 100 genes analyzed in FIG. 5 were evenly split between 50 genes that were up-regulated in tumor samples classified as mesenchymal cell-like, and 50 genes that are up-regulated in tumor samples classified as epithelial cell-like. The tumor samples were classified as mesenchymal cell-like or epithelial cell-like based on the PC1 score.

In addition, the analysis presented in FIG. 5 also tested for positive and negative correlations of gene expression levels for genes found in different multi-gene signatures such as the EMT Signature (described in Example 1, herein), TGF-beta (Singh et al., 2009, Cancer Cell 15:489-500), RAS (Bild et al., 2006, Nature 439:353-57), proliferation signature (Dai et al., 2005, Cancer Research 65:4059-66), MYC signature (Bild et al., 2006, Nature 439:353-57), and RAS signature (Bild et al., 2006, Nature 439:353-57). TGF-beta is a known driver of the EMT program (Singh et al., 2009, Cancer Cell 15:489-500), thus it is not surprising that the TGF-beta signature correlates with both the PC1 and EMT signatures in FIG. 5. In contrast, RAS activation/dependency/addiction has been shown to anti-correlate with the EMT program (Singh et al., 2009, Cancer Cell 15:489-500). K-RAS dependent cells exhibit an epithelial morphology, expressing significant cortical CDH1 but little VIM. Conversely, RAS-independent cells express low levels of CDH1, but have high levels of VIM. The results presented in FIG. 5 are consistent with both of these findings. Of interest, the cellular proliferation signature (Dai et al., 2005, Cancer Research 65:4059-66), and an effecter of such, the MYC signature (Bild et al., 2006, Nature 439:353-57), both anti-correlate with the mesenchymal arms of the EMT Signature and PC1 Signature.

The biology of the about 5000 genes representing the “intrinsic” PC1 gene set first identified in Example 3, above, was not revealed by the standard functional analysis algorithms that often identify multiple biological pathways linked to complex gene expression signatures. In fact, analysis of the 5000 PC1 genes by Ingenuity, Kegg, and GeneGo algorithm approaches identified multiple potential biological pathways that might be responsible for the observed molecular subclassification (data not shown). This approach did not precisely clarify the biology behind the observed gene expression changes represented in PC1, but suggested that biological pathways related to cellular adhesion and an extracellular matrix were significantly affected.

To better describe the biological functionality of the PC1 Signature (TABLES 4A and 4B), about 300 additional lung cancer cell line-derived and lung cancer tumor-derived signatures were analyzed for their association with the PC1 Signature. These cell-line derived and tumor-derived signatures represent gene lists that were collected from multiple sources, wherein each gene list was made up of genes that were found to be statistically significant in a context in which they were derived. Gene selection for inclusion in the gene list was accomplished by either correlation to a biological meaningful endpoint, differential expression between known clinical subtypes, or a change in gene expression post-dose.

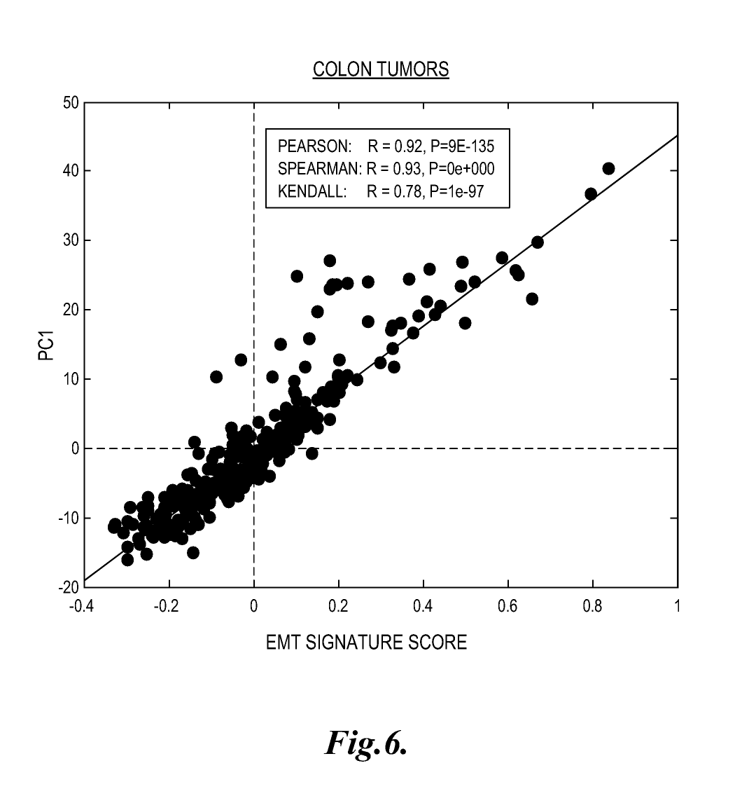

These analyses found a high correlation of the PC1 Signature with the lung cancer cell line derived EMT Signature as the most significantly associated (P<10⁻¹³⁵) with the PC1 Signature (FIG. 6). FIG. 6 shows a scatter plot comparing the values of EMT signature scores (x-axis) versus the values of PC1 (the first principal component) (y-axis) for each tumor sample in the dataset of 326 Moffitt colorectal cancer tumors. Importantly, as shown in FIG. 6, the mesenchymal and epithelial arms of the EMT signature were directionally correlated with the PC1 Signature mesenchymal and epithelial arms (P<10⁻¹⁶, Fisher Exact Test).

Another significant finding obtained from these data analysis results was that the unsupervised PC1 gene set (about 5000 genes), which represented an “intrinsic” subtype classifier of colon cancer, appears to be driven by genes within the EMT Signature (TABLES 2A and 2B). In fact, 92% of probes mapped to genes in the EMT mesenchymal arm were positively correlated with the PC1 Signature score and 82% of probes from genes in the EMT epithelial arm were negatively correlated with the PC1 Signature score, corresponding to Fisher exact test p-value of 2×10⁻¹⁶.

### Example 6

### PC1 and EMT Signature Scores Predict Disease Progression and Recurrence

Having identified PC1 Signature as an intrinsic gene expression signature closely linked to the EMT program; in this Example it is shown that the mesenchymal phenotype (i.e., high PC1 Signature Score and high EMT Signature Score), predicts recurrence of colon cancer.

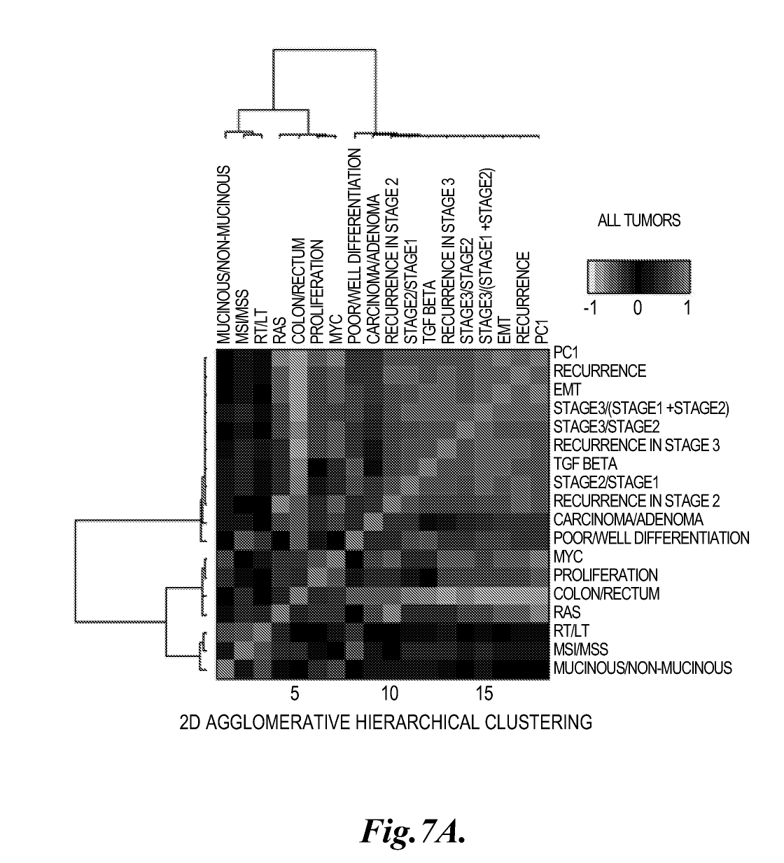

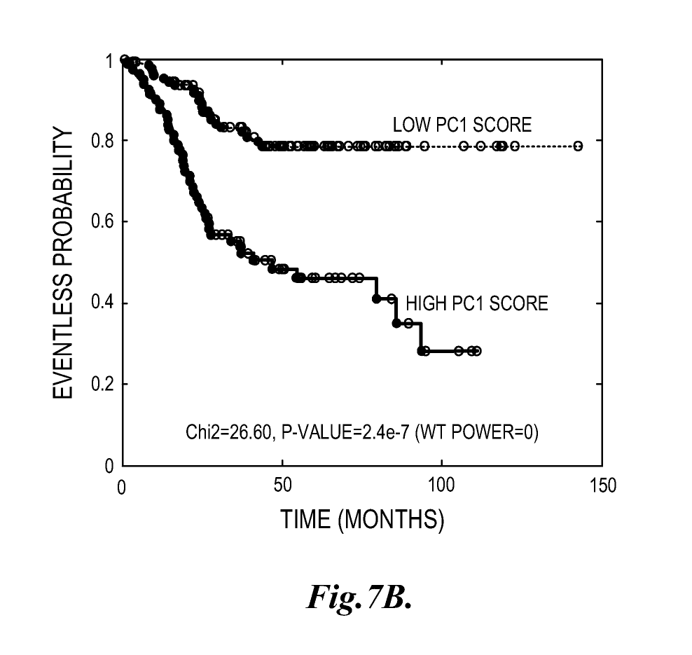

FIG. 7, Panel A, is a covariance matrix that demonstrates that the PC1 Signature Score correlates well (statistically significant with a p value<0.01) with the EMT Signature Score, with disease recurrence, disease progression, and differentiation status, but not with gene expression signatures linked to adenoma versus carcinoma, MSI status, or mucinous versus nonmucinous cancers based on comparison with the colon cancer gene expression signatures developed as described below. Moreover, PC1 Signature and EMT Signature scores both are anti-correlated with RAS (Bild et al., 2006, Nature 439:353-57), MYC (Bild et al., 2006, Nature 439:353-357), Proliferation (Dai et al., 2005, Cancer Research 65:4059-66), and colon laterality signatures. MYC and RAS signatures were obtained from Bild et al., Nature 439:353-357 (2006).

The colon cancer gene expression signatures used in the analysis shown in FIG. 7 were derived as follows.

Gene sets were identified that were associated with different endpoints related to tumor histology. Each comparison was carried out on non-metastatic samples with known stage, histology, and collection site. For each comparison, two gene sets (up and down regulated) were identified by t-test with p-value<0.01, split by a sign of fold change, selection of unique gene markers among 100 probes most differentially expressed by an absolute value of fold change. Performance of these marker sets was evaluated by back substitution and the scores for marker sets were computed as the mean of probes mapped by the marker to the up-regulated subset minus the mean of the probes that are mapped by the marker to the down-regulated subset. The marker sets were found to have ROC AUC>0.7 and 1-way ANOVA p-value<1e-6 when applied to distinguish the same samples that were used to identify these markers. A signature score for a given gene set was obtained by averaging the expression levels of the probes that mapped the marker to that gene set.

Gene expression signatures for each for the following scenarios was created: RT/LT: right/left colon cancer gene expression signature (also referred to as “laterality” was computed by comparing 60 samples collected in right (RT) colon versus 18 samples collected in left (LT) colon.

Mucinous/Non-mucinous colon carcinoma gene expression signature was developed by comparing 35 mucinous colon carcinoma samples versus 165 non-mucinous colon carcinoma samples.

MSI/MSS (Microsatellite instability/Microsatellite stable colon cancer) gene expression signature was created by comparing 6 MSI colon cancer samples versus 73 MSS colon cancer samples.

Carcinoma/Adenoma gene expression signature was created by comparing 22 pure colon adenocarcinoma samples versus 5 pure colon adenoma samples.

Poor/Well differentiation gene expression signature was developed by comparing 32 poorly differentiated colon cancer samples versus 19 well-differentiated colon cancer samples. Differentiation status information was obtained from the histology report.

Colon/Rectum gene expression signature was developed by comparing 50 tumor samples collected in colon versus 19 tumor samples collected in rectum.

Stage2/Stage1 gene expression signature was developed by comparing 59 colon cancer samples from stage 2 patients versus 32 colon cancer samples obtained from stage 1 patients.

Stage3/Stage2 gene expression signature was developed by comparing 71 colon cancer samples obtained from stage 3 patients versus 59 colon cancer samples obtained from stage 2 patients.

Recurrence gene expression signatures (recurrence in Stage 2, recurrence in Stage 3), were generated based on the genes that were found to have statistically significant differential expression levels between tumor samples of a given stage (i.e., Stage 1, Stage 2, Stage 3, or Stage 4) in patients that did not experience a tumor recurrence within a 3-year period. For each comparison, two sets of genes were generated (up-regulated expression levels in tumor samples from patients suffering from recurrence and down-regulated expression levels in tumor samples from patients suffering from recurrence), and the scores were computed as the difference in the mean probe intensities for these two gene sets.

FIG. 7, panel B, is a Kaplan-Meier Curve of disease-free survival time of colon cancer patients (stages 1, 2, 3, and 4) from which the 326 colorectal tumors from the Moffitt dataset were derived, with the tumor samples stratified into two groups based on whether the PC1 score was below or above the mean, showing eventless probability (y-axis) plotted against time measured in months (x-axis), showing that a low PC1 score correlates with a good colon cancer prognosis, and a high PC1 score correlates with a poor colon cancer prognosis. The results shown in FIG. 7 demonstrate that the PC1 Signature, despite being developed with an unsupervised approach, is capable of differentiating good (i.e., low PC1 Signature score) from poor (i.e., high PC1 Signature score) colon cancer prognosis.

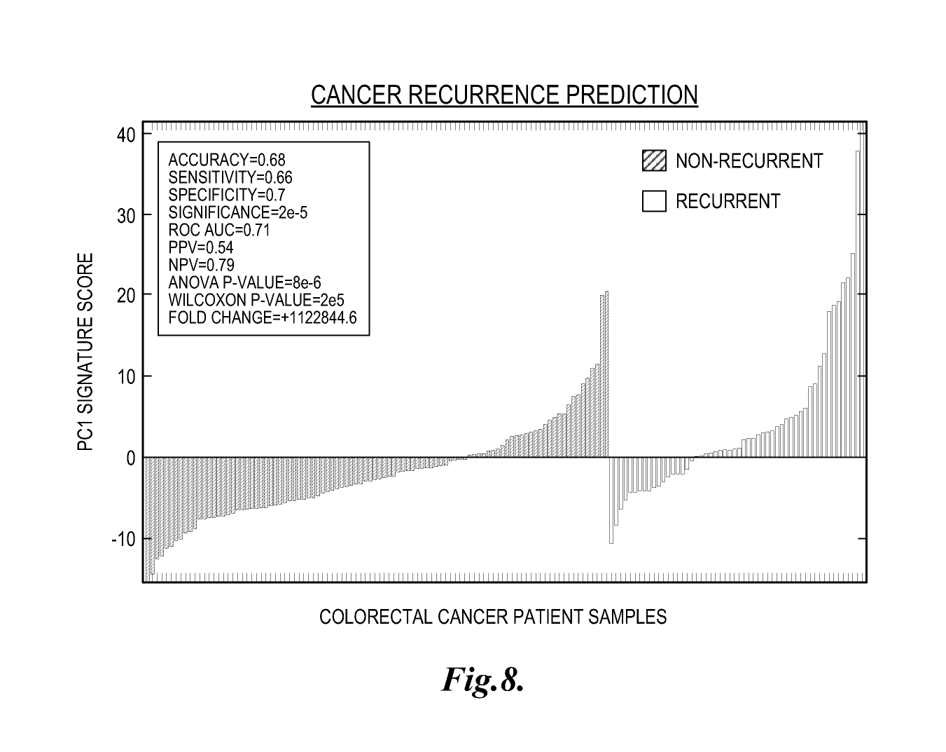

In addition, FIG. 8, which shows a waterfall plot of recurrence prediction for the Moffitt Colorectal cancer tumor samples (stagemm2 and stage 3), shows that human patients with a high PC1 Signature score were correlated with recurrence of colon cancer, whereas those patients with a low PC1 Signature score were more likely to be non-recurrent. The results shown in FIG. 8 have a confusion matrix: TP=37, FP=31, FN=19, TN=71; plotted value=input value−adjustment, adjustment=−0.86188). Cancer recurrence patients versus non-recurrent patients are defined based on the presence of recurrent disease (metastasis) within a three year time frame.

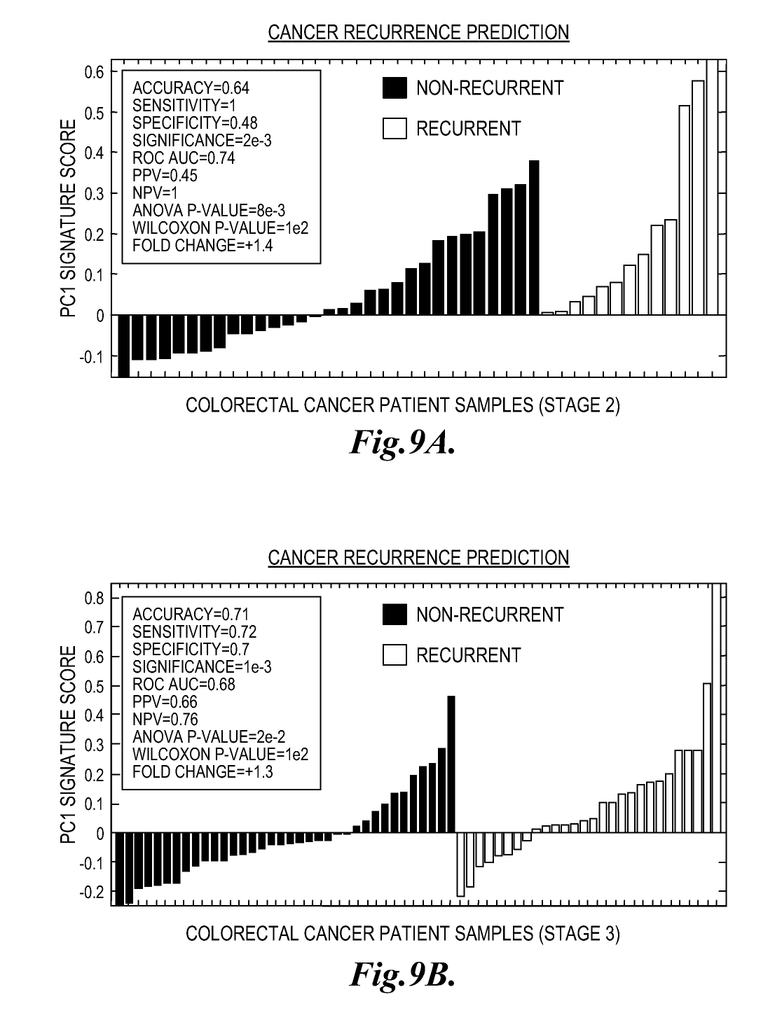

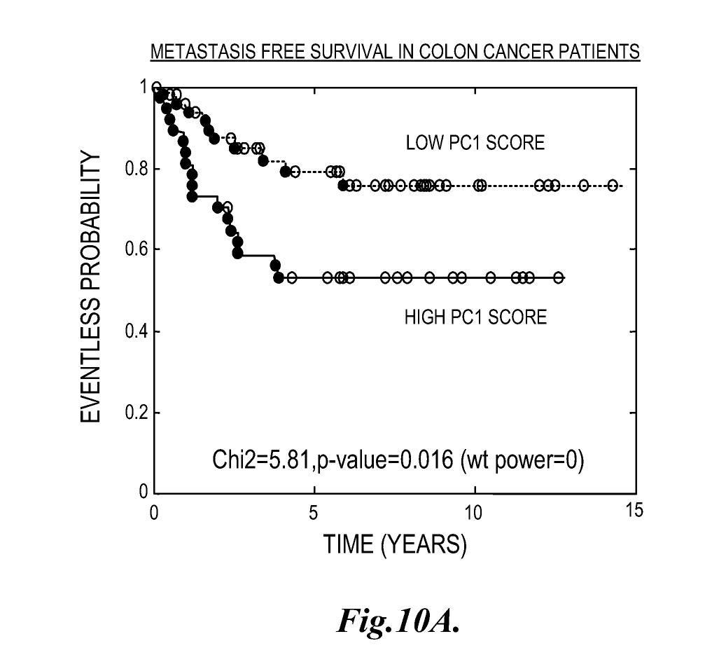

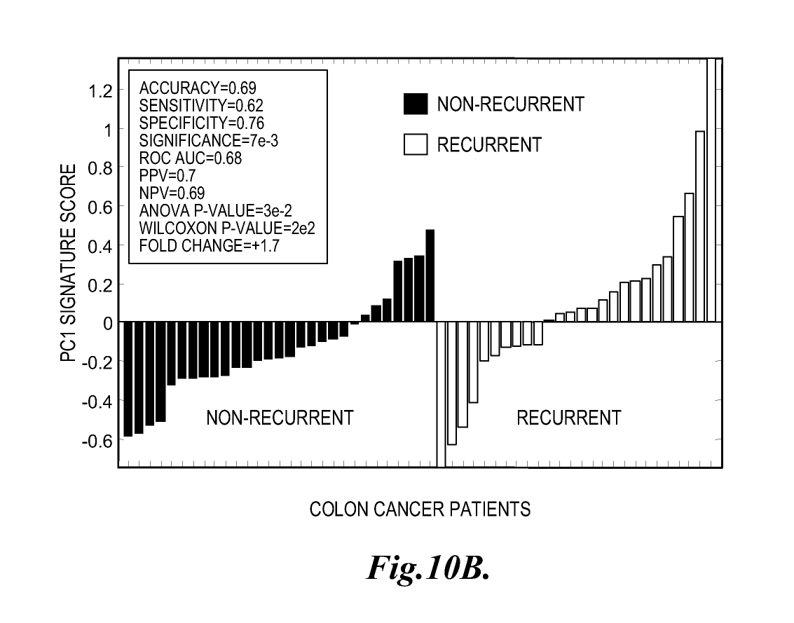

FIG. 9, further extends the results shown in FIG. 8, and shows a waterfall plot of cancer recurrence prediction using the PC1 Signature score for patients who contributed samples used to generate the Moffitt Cancer Center colorectal cancer gene expression dataset. Panel A shows patients' samples classified as Stage 2 colorectal cancer. The results shown in FIG. 9A have a confusion matrix: TP=13, FP=16, FN=0, TN=15, plotted value=input value−adjustment, adjustment=−0.09586). Panel B shows patients' samples classified as Stage 3 colorectal cancer. The results shown in FIG. 9B have a confusion matrix: TP=21, FP=11, FN=8, TN=26, plotted value=input value—adjustment, adjustment=−0.031702. Cancer recurrence and non-recurrent patients are defined as described for FIG. 8. The results in FIG. 9 show that a high PC1 Signature score correlates with recurrence of colon cancer even for intermediate Stage II (FIG. 9, Panel A) and Stage III (FIG. 9, Panel B) Importantly, the PC1 Signature score was also predictive of poor patient outcome in two completely independent data sets. In a data set from the Netherlands Cancer Institute (NKI), the PC1 Signature score predicted metastasis free survival (FIG. 10, Panel A) in 118 colon cancer patients (Stages 2 and 3). FIG. 10A is a Kaplan-Meier Curve of metastasis-free survival time of colon cancer patients (stages 2 and 3) showing metastasis-free survival time (y-axis) plotted against time (measured in years) (x-axis), showing that a low PC1 score correlates with a good colon cancer prognosis (i.e., a lower likelihood of metastasis), and a high PC1 score correlates with a poor colon cancer prognosis (i.e., a higher likelihood of metastasis).

As shown in FIG. 10A, Colon cancer patients in the NM study having a low PC1 signature score were more likely to stay metastasis free than patients having a high PC1 signature score. FIG. 10A shows a Kaplan-Meier Curve of metastasis-free survival time of colon cancer patients (stages 2 and 3) showing metastasis-free survival time (recurrence-free time) (y-axis) plotted against time (measured in years). The PC1 Score was computed as the difference in mean intensities for the genes that were most positively and negatively correlated to PC1 in the Moffitt colorectal dataset of 326 tumors. The samples were stratified into two groups: “high PC1 Score” or “low PC1 score” depending on whether their PC1 score was above or below the mean PC1 Score on the given dataset. Similarly, in another colorectal cancer dataset of 55 patients, referred to as the German colorectal cancer data set (Lin et al., 2007, Clin. Cancer Res. 13:498-507), patients having a low PC1 signature score were more likely to remain disease free, i.e., non-recurrent, as compared to patients having a high PC1 signature score (FIG. 10, Panel B). The results shown in FIG. 10B have a confusion matrix: TP=16, FP=7, FN=10, TN=22, plotted value=input value−adjustment, adjustment−0.032787.

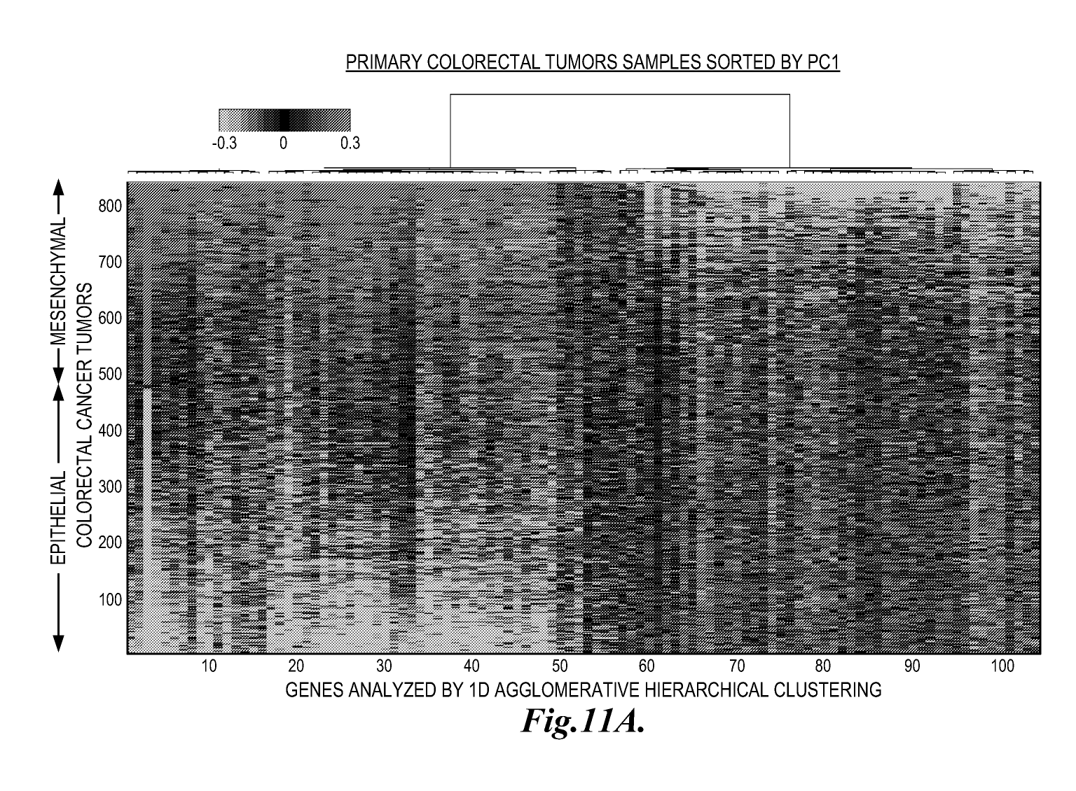

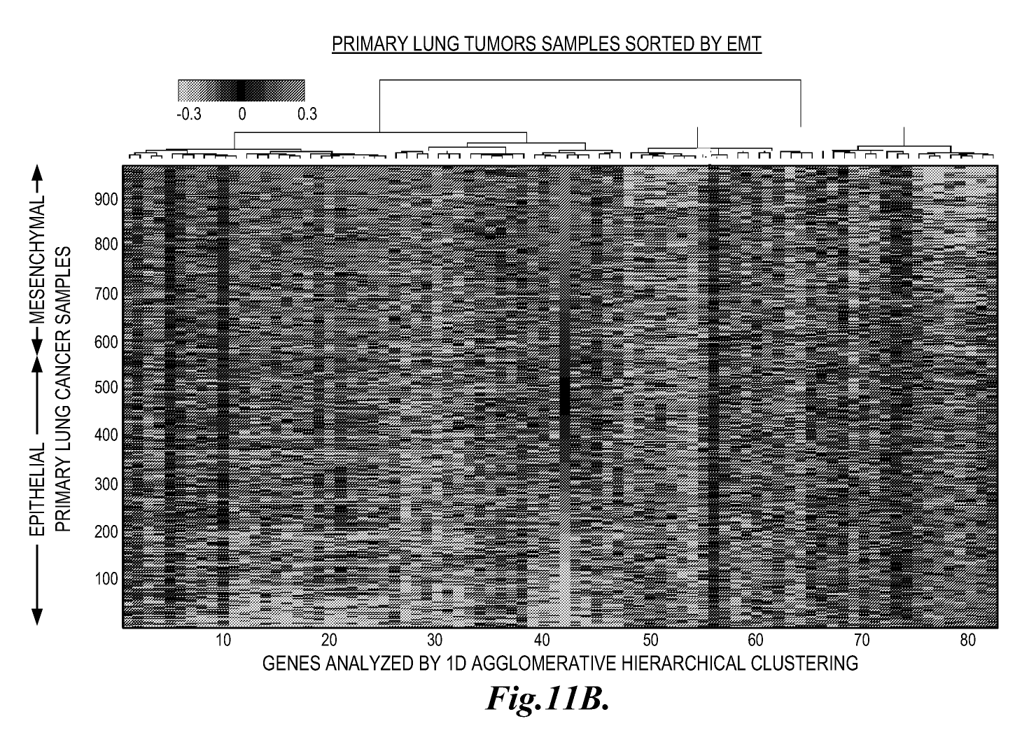

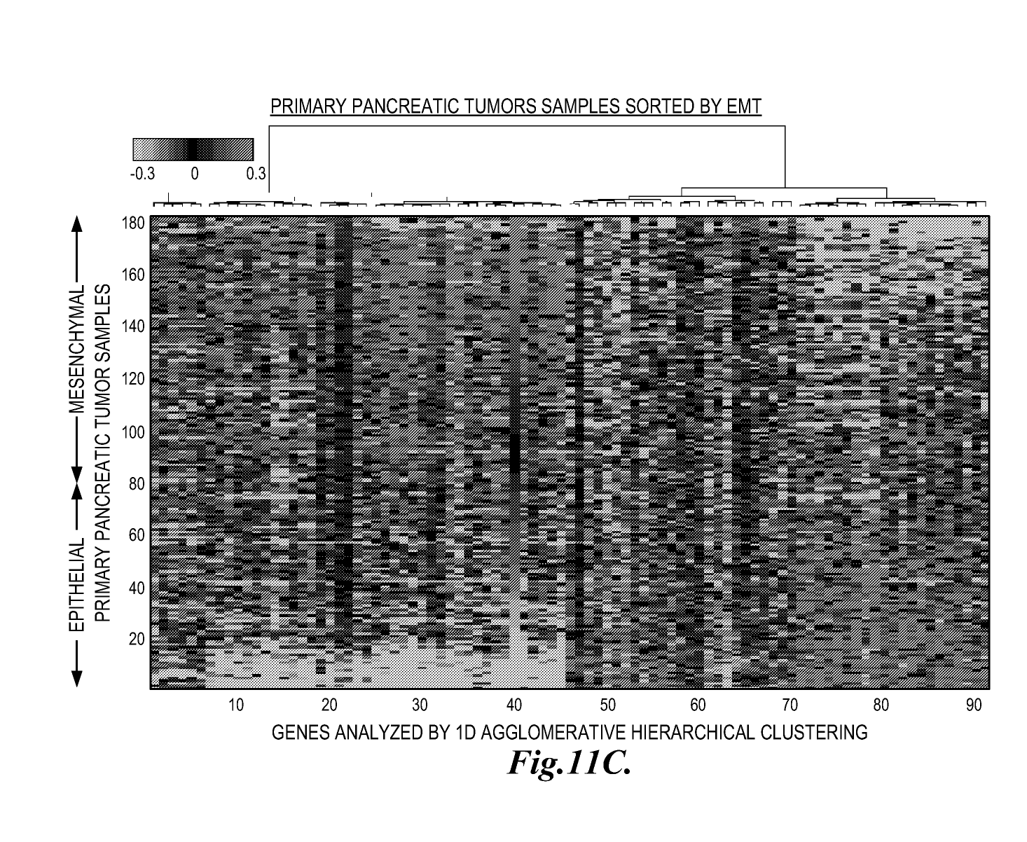

FIG. 11 shows gene expression profiling stratified by PC1 signature score (Panel A) or EMT Signature Score (Panels B and C) for three different cancers (colorectal, lung, and pancreatic cancer) having different cancer recurrence rates.

FIG. 11, Panel A shows expression profiles obtained from 830 primary colorectal tumor samples, obtained from the Merck-Moffitt collaboration program, stratified by PC1 signature score. TABLE 6 shows the gene symbols of the 104 genes/gene signatures analyzed, corresponding to positions 1 to 104 shown across the top of FIG. 11A. Genes positively correlated with a PC1 Signature score are shown as red (darker greyscale) in FIG. 11A, and shown in TABLE 6 as mesenchymal up-regulated (M). Genes negatively correlated with a PC1 Signature score are shown as blue (lighter greyscale) in FIG. 11A, and shown in TABLE 6 as epithelial up-regulated (E). The 104 genes included in this analysis were chosen based on a literature search, and are ordered in TABLE 6 and FIG. 11A based on the similarity of their gene expression profiles and PC1 score.

TABLE 6
Individual Genes And Signatures Of Genes Analyzed In FIG. 11a

| Reference number | | Type: | Upregulated in |
|---|---|---|---|
| with regard | | individual | Mesenchymal (M) |
| to FIG. 11A | Gene or Gene | or gene | or in Epithelial (E) |
| (horizontal) | Signature | signature | in FIG. 11A |
| 1 | SH2D3C | Individual | M |
| 2 | TGFbeta | Signature | M |
| 3 | PC1 | Signature | M |
| 4 | EMT | Signature | M |
| 5 | GLIS2 | Individual | M |
| 6 | GLI3 | Individual | M |
| 7 | FGFR1 | Individual | M |
| 8 | MAP3K3 | Individual | M |
| 9 | TWIST2 | Individual | M |
| 10 | FBLN1 | Individual | M |
| 11 | CDON | Individual | M |
| 12 | TAGLN | Individual | M |
| 13 | TGFB1I1 | Individual | M |
| 14 | VEGFB | Individual | M |
| 15 | LAMB2 | Individual | M |
| 16 | NFIC | Individual | M |
| 17 | EPHA3 | Individual | M |
| 18 | WASF3 | Individual | M |
| 19 | SFRP1 | Individual | M |
| 20 | SRPX | Individual | M |
| 21 | TIAM1 | Individual | M |
| 22 | MMRN2 | Individual | M |
| 23 | MGP | Individual | M |
| 24 | FBLN5 | Individual | M |
| 25 | ARMCX1 | Individual | M |
| 26 | RECK | Individual | M |
| 27 | ZFPM2 | Individual | M |
| 28 | FLRT2 | Individual | M |
| 29 | TCF4 | Individual | M |
| 30 | DZIP1 | Individual | M |
| 31 | CTGF | Individual | M |
| 32 | MSN | Individual | M |
| 33 | VIM | Individual | M |
| 34 | FOXC2 | Individual | M |
| 35 | MEOX2 | Individual | M |
| 36 | FGF1 | Individual | M |
| 37 | MRAS | Individual | M |
| 38 | AXL | Individual | M |
| 39 | GLI2 | Individual | M |
| 40 | ASPN | Individual | M |
| 41 | ECM2 | Individual | M |
| 42 | SPARC | Individual | M |
| 43 | HTRA1 | Individual | M |
| 44 | SNAI2 | Individual | M |
| 45 | TWIST1 | Individual | M |
| 46 | WISP1 | Individual | M |
| 47 | FN1 | Individual | M |
| 48 | CDH2 | Individual | M |
| 49 | FOXC1 | Individual | M |
| 50 | SLC39A6 | Individual | M |
| 51 | STX2 | Individual | M |
| 52 | ETV5 | Individual | M |
| 53 | SMAD1 | Individual | M |
| 54 | TGFBR1 | Individual | M |
| 55 | ACVR1 | Individual | M |
| 56 | RNF11 | Individual | M |
| 57 | SMAD3 | Individual | M |
| 58 | CLDN9 | Individual | E |
| 59 | SHH | Individual | E |
| 60 | PROLIFERATION | Signature | E |
| 61 | MYC | Signature | E |
| 62 | KAZALD1 | Individual | E |
| 63 | RSL1D1 | Individual | E |
| 64 | CD44 | Individual | E |
| 65 | LYPD5 | Individual | E |
| 66 | LCN2 | Individual | E |
| 67 | S100P | Individual | E |
| 68 | RAS | Signature | E |
| 69 | MST1R | Individual | E |
| 70 | SFN | Individual | E |
| 71 | KRT19 | Individual | E |
| 72 | ITGB4 | Individual | E |
| 73 | SDC1 | Individual | E |
| 74 | TNS4 | Individual | E |
| 75 | MET | Individual | E |
| 76 | KRT8 | Individual | E |
| 77 | FOXA2 | Individual | E |
| 78 | CEACAM1 | Individual | E |
| 79 | CD24 | Individual | E |
| 80 | TMPRSS4 | Individual | E |
| 81 | PRSS8 | Individual | E |
| 82 | SOX9 | Individual | E |
| 83 | RBM35A | Individual | E |
| 84 | MAL2 | Individual | E |
| 85 | CLDN7 | Individual | E |
| 86 | CDH1 | Individual | E |
| 87 | CLDN4 | Individual | E |
| 88 | ELF3 | Individual | E |
| 89 | JUP | Individual | E |
| 90 | MMP15 | Individual | E |
| 91 | CRB3 | Individual | E |
| 92 | SPINT1 | Individual | E |
| 93 | PKP3 | Individual | E |
| 94 | RBM35B | Individual | E |
| 95 | IHH | Individual | E |
| 96 | ETS2 | Individual | E |
| 97 | ISX | Individual | E |
| 98 | FOXD2 | Individual | E |
| 99 | CDX1 | Individual | E |
| 100 | CDX2 | Individual | E |
| 101 | KIAA0152 | Individual | E |
| 102 | EPHB3 | Individual | E |
| 103 | DSC2 | Individual | E |
| 104 | EVI1 | Individual | E |

FIG. 11, Panel B shows expression profiles obtained from 950 primary lung tumor samples, obtained from the Merck-Moffitt collaboration program, stratified by EMT signature score. TABLE 7 shows the gene symbols of the 82 genes/gene signatures analyzed, corresponding to positions 1 to 82 across the top of FIG. 11B. Genes positively correlated with an EMT Signature score are shown as red (darker greyscale) in FIG. 11B and shown in TABLE 7 as mesenchymal up-regulated (M). Genes negatively correlated with an EMT Signature score are shown as blue (lighter greyscale) in FIG. 11B and shown in TABLE 7 and epithelial up-regulated (E). The 82 genes included in this analysis were chosen based on a literature search, and are ordered in TABLE 7 and FIG. 11B based on the similarity of their gene expression profiles and PC1 score.

TABLE 7
Individual Genes and Signatures of Genes Analyzed in FIG. 11B

| Reference number | | Type: | Upregulated in |
|---|---|---|---|
| with regard | | individual | Mesenchymal (M) |
| to FIG. 11B | Gene or Gene | or gene | or in Epithelial (E) |
| (horizontal) | Signature | signature | in FIG. 11B |
| 1 | SH2D3C | Individual | M |
| 2 | MAP3K3 | Individual | M |
| 3 | MGP | Individual | M |
| 4 | FBLN5 | Individual | M |
| 5 | MSN | Individual | M |
| 6 | STX2 | Individual | M |
| 7 | ARMCX1 | Individual | M |
| 8 | MRAS | Individual | M |
| 9 | AXL | Individual | M |
| 10 | VIM | Individual | M |
| 11 | FN1 | Individual | M |
| 12 | FLRT2 | Individual | M |
| 13 | SRPX | Individual | M |
| 14 | MMRN2 | Individual | M |
| 15 | TAGLN | Individual | M |
| 16 | FBLN1 | Individual | M |
| 17 | HTRA1 | Individual | M |
| 18 | FGF1 | Individual | M |
| 19 | CTGF | Individual | M |
| 20 | ASPN | Individual | M |
| 21 | SPARC | Individual | M |
| 22 | ECM2 | Individual | M |
| 23 | ZFPM2 | Individual | M |
| 24 | RECK | Individual | M |
| 25 | MEOX2 | Individual | M |
| 26 | CDON | Individual | M |
| 27 | CDH2 | Individual | M |
| 28 | EPHA3 | Individual | M |
| 29 | WASF3 | Individual | M |
| 30 | SFRP1 | Individual | M |
| 31 | FOXC1 | Individual | M |
| 32 | FOXC2 | Individual | M |
| 33 | ETV5 | Individual | M |
| 34 | TGFBR1 | Individual | M |
| 35 | RNF11 | Individual | M |
| 36 | ACVR1 | Individual | M |
| 37 | SLC39A6 | Individual | M |
| 38 | SMAD1 | Individual | M |
| 39 | WISP1 | Individual | M |
| 40 | TGFbeta | Signature | M |
| 41 | SNAI2 | Individual | M |
| 42 | EMT | Signature | M |
| 43 | DZIP1 | Individual | M |
| 44 | TCF4 | Individual | M |
| 45 | CD44 | Individual | E |
| 46 | LYPD5 | Individual | E |
| 47 | TIAM1 | Individual | M |
| 48 | TMPRSS4 | Individual | E |
| 49 | KRT19 | Individual | E |
| 50 | JUP | Individual | E |
| 51 | PKP3 | Individual | E |
| 52 | SFN | Individual | E |
| 53 | ITGB4 | Individual | E |
| 54 | TNS4 | Individual | E |
| 55 | PROLIFERATION | Signature | E |
| 56 | MYC | Signature | E |
| 57 | KAZALD1 | Individual | E |
| 58 | GLI2 | Individual | M |
| 59 | EPHB3 | Individual | E |
| 60 | CDX1 | Individual | E |
| 61 | CDX2 | Individual | E |
| 62 | ETS2 | Individual | E |
| 63 | CD24 | Individual | E |
| 64 | SOX9 | Individual | E |
| 65 | DSC2 | Individual | E |
| 66 | NFIC | Individual | M |
| 67 | ISX | Individual | E |
| 68 | KIAA0152 | Individual | E |
| 69 | FOXD2 | Individual | E |
| 70 | KRT8 | Individual | E |
| 71 | CLDN9 | Individual | E |
| 72 | SHH | Individual | E |
| 73 | IHH | Individual | E |
| 74 | FOXA2 | Individual | E |
| 75 | SPINT1 | Individual | E |
| 76 | CLDN4 | Individual | E |
| 77 | ELF3 | Individual | E |
| 78 | MST1R | Individual | E |
| 79 | MMP15 | Individual | E |
| 80 | PRSS8 | Individual | E |
| 81 | RBM35B | Individual | E |
| 82 | CRB3 | Individual | E |

FIG. 11, Panel C shows expression profiles obtained from 180 primary pancreatic tumor samples, obtained from the Merck-Moffitt collaboration program, stratified by EMT signature score. TABLE 8 shows the gene symbols of the 92 genes/gene signatures analyzed, corresponding to positions 1 to 92 across the top of FIG. 11C. Genes positively correlated with an EMT Signature score are shown as red (darker greyscale) in FIG. 11C and shown in TABLE 8 as mesenchymal up-regulated (M). Genes negatively correlated with an EMT Signature score are shown as blue (lighter greyscale) in FIG. 11C, and shown in TABLE 8 as epithelial up-regulated (E). The 92 genes included in this analysis were chosen based on a literature search, and are ordered in TABLE 8 and FIG. 11C based on the similarity of their gene expression profiles and PC1 score.

TABLE 8
Individual Genes and Signatures of Genes Analyzed in FIG. 11C

| Reference number | | Type: | Upregulated in |
|---|---|---|---|
| with regard | | individual | Mesenchymal (M) |
| to FIG. 11C | Gene or Gene | or gene | or in Epithelial (E) |
| (horizontal) | Signature | signature | in FIG. 11C |
| 1 | ETV5 | Individual | M |
| 2 | TGFBR1 | Individual | M |
| 3 | RNF11 | Individual | M |
| 4 | ACVR1 | Individual | M |
| 5 | SLC39A6 | Individual | M |
| 6 | SMAD1 | Individual | M |
| 7 | GLI2 | Individual | M |
| 8 | GLIS2 | Individual | M |
| 9 | TWIST1 | Individual | M |
| 10 | TAGLN | Individual | M |
| 11 | GLI3 | Individual | M |
| 12 | AXL | Individual | M |
| 13 | HTRA1 | Individual | M |
| 14 | CDH2 | Individual | M |
| 15 | FGF1 | Individual | M |
| 16 | TGFbeta | Signature | M |
| 17 | WISP1 | Individual | M |
| 18 | FN1 | Individual | M |
| 19 | STX2 | Individual | M |
| 20 | MRAS | Individual | M |
| 21 | MSN | Individual | M |
| 22 | VIM | Individual | M |
| 23 | SNAI2 | Individual | M |
| 24 | TIAM1 | Individual | M |
| 25 | MGP | Individual | M |
| 26 | FBLN5 | Individual | M |
| 27 | ZFPM2 | Individual | M |
| 28 | RECK | Individual | M |
| 29 | FBLN1 | Individual | M |
| 30 | ASPN | Individual | M |
| 31 | SPARC | Individual | M |
| 32 | CTGF | Individual | M |
| 33 | EPHA3 | Individual | M |
| 34 | SFRP1 | Individual | M |
| 35 | TWIST2 | Individual | M |
| 36 | CDON | Individual | M |
| 37 | WASF3 | Individual | M |
| 38 | FLRT2 | Individual | M |
| 39 | DZIP1 | Individual | M |
| 40 | EMT | Signature | M |
| 41 | SRPX | Individual | M |
| 42 | ARMCX1 | Individual | M |
| 43 | TCF4 | Individual | M |
| 44 | ECM2 | Individual | M |
| 45 | MEOX2 | Individual | M |
| 46 | PROLIFERATION | Signature | M |
| 47 | MYC | Signature | M |
| 48 | FOXD2 | Individual | E |
| 49 | ETS2 | Individual | E |
| 50 | CDX1 | Individual | E |
| 51 | ISX | Individual | E |
| 52 | CDX2 | Individual | E |
| 53 | KIAA0152 | Individual | E |
| 54 | EPHB3 | Individual | E |
| 55 | KAZALD1 | Individual | E |
| 56 | KRT8 | Individual | E |
| 57 | CLDN9 | Individual | E |
| 58 | IHH | Individual | E |
| 59 | SHH | Individual | E |
| 60 | FOXA2 | Individual | E |
| 62 | FOXC1 | Individual | M |
| 63 | SMAD3 | Individual | M |
| 64 | FOXC2 | Individual | M |
| 65 | MAP3K3 | Individual | M |
| 66 | LAMB2 | Individual | M |
| 67 | CD44 | Individual | E |
| 68 | LYPD5 | Individual | E |
| 69 | NFIC | Individual | M |
| 70 | MMRN2 | Individual | M |
| 71 | DSC2 | Individual | E |
| 72 | ITGB4 | Individual | E |
| 73 | KRT19 | Individual | E |
| 74 | MST1R | Individual | E |
| 75 | JUP | Individual | E |
| 76 | PKP3 | Individual | E |
| 77 | RAS | Signature | E |
| 78 | SFN | Individual | E |
| 79 | TNS4 | Individual | E |
| 80 | CEACAM1 | Individual | E |
| 81 | CRB3 | Individual | E |
| 82 | MMP15 | Individual | E |
| 83 | CLDN4 | Individual | E |
| 84 | CLDN7 | Individual | E |
| 85 | LCN2 | Individual | E |
| 86 | SPINT1 | Individual | E |
| 87 | PRSS8 | Individual | E |
| 88 | ELF3 | Individual | E |
| 89 | RBM35B | Individual | E |
| 90 | CD24 | Individual | E |
| 91 | SOX9 | Individual | E |
| 92 | EVI1 | Individual | E |

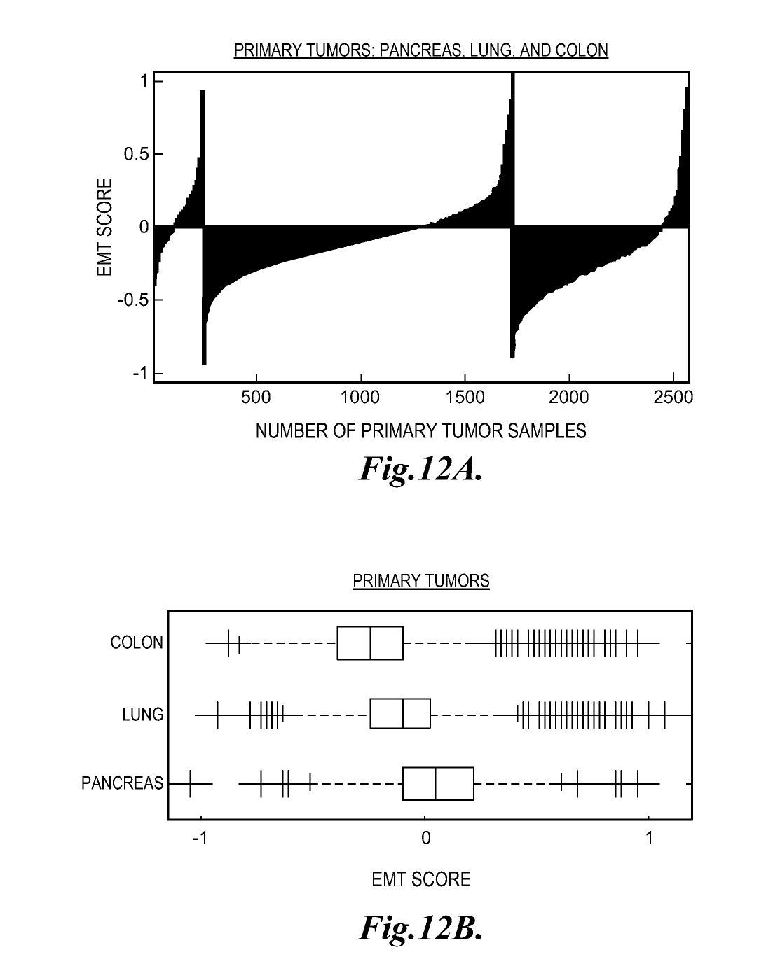

FIG. 12, Panel A shows a summary of the pancreas, lung, and colon gene expression profiling datasets presented in FIG. 11, sorted by cancer type and EMT Signature scores. The x-axis shows primary tumor samples grouped by the cancer type (pancreas, lung, colon) and sorted within each cancer type by the EMT signature score. FIG. 12, Panel B shows a boxplot analysis of the differential EMT signature scores for the three cancer types (colon<lung<pancreas) following normalization across all patient samples. These data summary figures shows that there was a clear difference between the average colon, lung, and pancreas cancers' EMT Signature scores, with colon having a lower average EMT signature score than lung cancer, which was lower than pancreatic cancer. This order of cancer EMT Signature scores correlates with the observed disease recurrence rates for these cancers. This shows that, in general, EMT Signature scores can be used to predict likelihood of cancer recurrence.

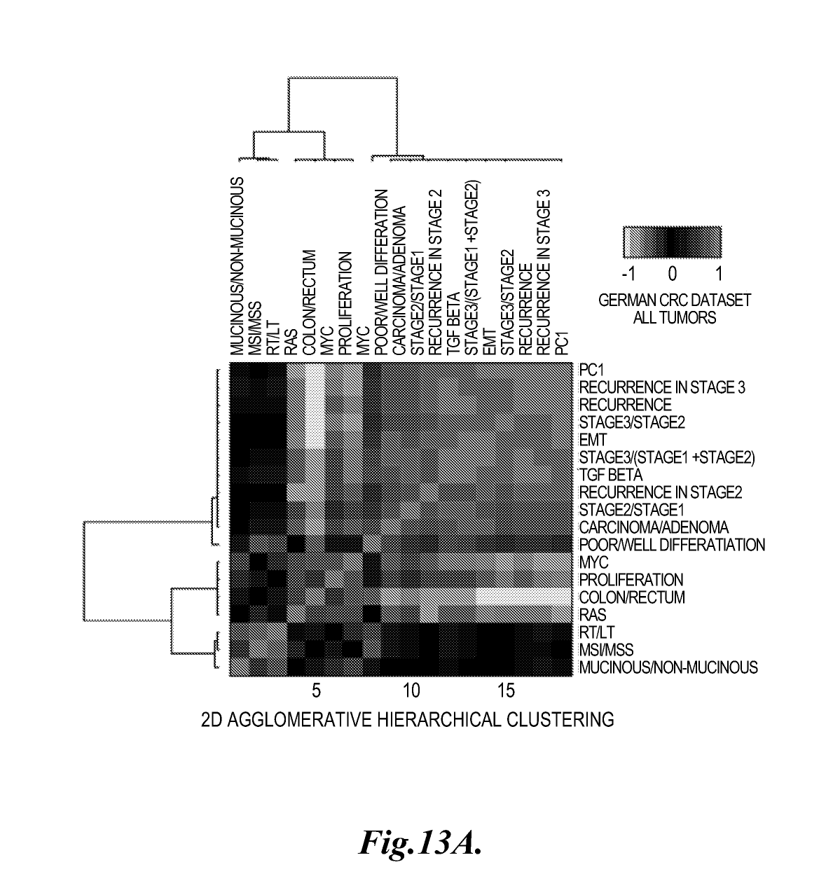

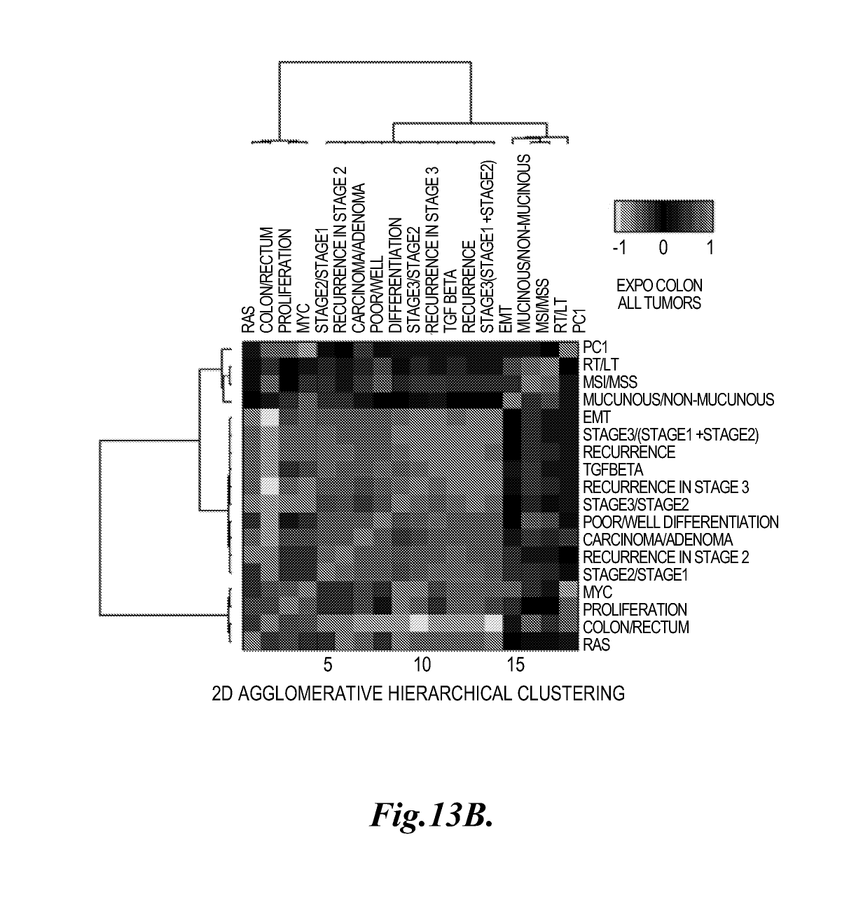

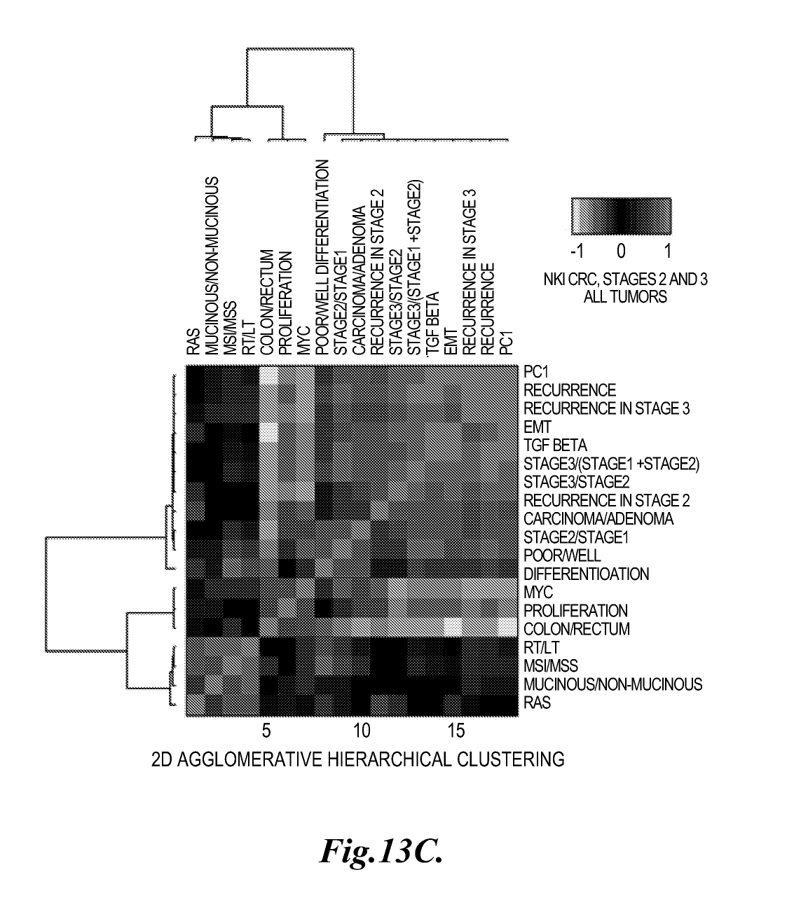

FIG. 13 shows covariance matrices for other colorectal datasets similar to that shown in FIG. 7, Panel A, for the Moffitt colorectal cancer dataset. FIG. 13, Panel A shows a covariance matrix using the German colorectal cancer dataset (Lin et al., 2007, Clin. Cancer Res. 13:498-507) (see also FIG. 10B). FIG. 13, Panel B, shows a covariance matrix using a colon cancer dataset from ExPO, which is publicly accessible at Expression Project of Oncology (ExPO), Series GSE2109, at ncbi.nlm.nih.gov/projects/geo/query/acc.cgi?acc=GSE2109 (see also FIG. 4). FIG. 13, Panel C, shows a covariance matrix using a colon cancer dataset obtained from 118 CRC samples from the Netherlands Cancer Institute (NKI) (see also FIG. 10, Panel A). These covariance data analyses results show that PC1 Signature scores and EMT Signature scores show the same pattern of covariance to disease and other cancer-related signature score endpoints, as observed in FIG. 7, Panel A, for the Moffitt colorectal cancer dataset. Taken together, these covariance matrices data show that PC1 Signature scores and EMT Signature scores are correlated to cancer progression and to poor differentiation status of cancer tumors.

### Example 7

### PC1 and EMT Signature Scores Are Correlated With Specific MicroRNA Levels

Expression levels of about 700 microRNAs were measured in about 70 Stage I-IV human colon cancers with a global microRNA platform that had been previously assessed by microarray analysis. Out of these about 70 samples, 49 samples were selected and subsequently used for the analysis after data processing and quality control threshold criteria were imposed. TABLE 9A shows the top 74 miRNAs (SEQ ID NOS:509-582) that were identified from the 700 miRNAs tested which are positively correlated with EMT/PC1 Signature scores and have a rho score by Pearson analysis of 20% or higher, sorted by the EMT p-value (Pearson).

TABLE 9A
MicroRNAS Positively Correlated to EMT Signature Score

| | EMT | EMT | |
|---|---|---|---|
| | rho | p-value | SEQ |
| Micro RNA Measured | Pearson | Pearson | ID NO: |
| has-miR-212-4373087 (FAM, NFQ) | 46% | 1.E−03 | 509 |
| hsa-miR-214-4395417 (FAM, NFQ) | 40% | 5.E−03 | 510 |
| hsa-miR-132-4373143 (FAM, NFQ) | 39% | 5.E−03 | 511 |
| hsa-miR-671-3p-4395433 (FAM, NFQ) | 38% | 7.E−03 | 512 |
| hsa-miR-99a-4373008 (FAM, NFQ) | 38% | 7.E−03 | 513 |
| hsa-miR-100-4373160 (FAM, NFQ) | 37% | 8.E−03 | 514 |
| hsa-miR-193b-4395478 (FAM, NFQ) | 36% | 1.E−02 | 515 |
| hsa-miR-539-4378103 (FAM, NFQ) | 35% | 1.E−02 | 516 |
| hsa-miR-24-4373072 (FAM, NFQ) | 35% | 1.E−02 | 517 |
| hsa-miR-489-4395469 (FAM, NFQ) | 35% | 2.E−02 | 518 |
| hsa-miR-125b-1*-4395489 (FAM, NFQ) | 35% | 2.E−02 | 519 |
| hsa-miR-433-4373205 (FAM, NFQ) | 34% | 2.E−02 | 520 |
| hsa-miR-432-4373280 (FAM, NFQ) | 34% | 2.E−02 | 521 |
| hsa-miR-342-3p-4395371 (FAM, NFQ) | 33% | 2.E−02 | 522 |
| hsa-miR-506-4373231 (FAM, NFQ) | 33% | 2.E−02 | 523 |
| hsa-miR-139-5p-4395400 (FAM, NFQ) | 33% | 2.E−02 | 524 |
| hsa-miR-542-5p-4395351 (FAM, NFQ) | 33% | 2.E−02 | 525 |
| hsa-miR-125b-4373148 (FAM, NFQ) | 33% | 2.E−02 | 526 |
| hsa-miR-493-4395475 (FAM, NFQ) | 32% | 2.E−02 | 527 |
| hsa-miR-99b*-4395307 (FAM, NFQ) | 32% | 2.E−02 | 528 |
| hsa-miR-193a-3p-4395361 (FAM, NFQ) | 32% | 2.E−02 | 529 |
| hsa-miR-99a*-4395252 (FAM, NFQ) | 32% | 3.E−02 | 530 |
| hsa-miR-30a*-4373062 (FAM, NFQ) | 31% | 3.E−02 | 531 |
| hsa-miR-9-4373285 (FAM, NFQ) | 31% | 3.E−02 | 532 |
| hsa-miR-892b-4395325 (FAM, NFQ) | 31% | 3.E−02 | 533 |
| hsa-miR-888-4395323 (FAM, NFQ) | 31% | 3.E−02 | 534 |
| hsa-miR-365-4373194 (FAM, NFQ) | 30% | 4.E−02 | 535 |
| hsa-miR-152-4395170 (FAM, NFQ) | 30% | 4.E−02 | 536 |
| hsa-let-7c-4373167 (FAM, NFQ) | 29% | 4.E−02 | 537 |
| hsa-miR-150-4373127 (FAM, NFQ) | 29% | 4.E−02 | 538 |
| hsa-miR-502-3p-4395194 (FAM, NFQ) | 29% | 4.E−02 | 539 |
| hsa-miR-140-5p-4373374 (FAM, NFQ) | 28% | 5.E−02 | 540 |
| hsa-miR-193a-5p-4395392 (FAM, NFQ) | 28% | 5.E−02 | 541 |
| hsa-miR-193b*-4395477 (FAM, NFQ) | 28% | 5.E−02 | 542 |
| hsa-miR-25*-4395553 (FAM, NFQ) | 27% | 6.E−02 | 543 |
| hsa-miR-541-4395312 (FAM, NFQ) | 27% | 6.E−02 | 544 |
| hsa-miR-134-4373299 (FAM, NFQ) | 27% | 6.E−02 | 545 |
| hsa-miR-9*-4395342 (FAM, NFQ) | 27% | 6.E−02 | 546 |
| hsa-miR-188-5p-4395431 (FAM, NFQ) | 27% | 6.E−02 | 547 |
| hsa-miR-222-4395387 (FAM, NFQ) | 27% | 6.E−02 | 548 |
| hsa-miR-30e*-4373057 (FAM, NFQ) | 27% | 6.E−02 | 549 |
| hsa-miR-125a-5p-4395309 (FAM, NFQ) | 27% | 6.E−02 | 550 |
| hsa-miR-520e-4373255 (FAM, NFQ) | 27% | 7.E−02 | 551 |
| hsa-miR-199a-3p-4395415 (FAM, NFQ) | 26% | 7.E−02 | 552 |
| hsa-miR-127-5p-4395340 (FAM, NFQ) | 26% | 8.E−02 | 553 |
| hsa-miR-410-4378093 (FAM, NFQ) | 25% | 8.E−02 | 554 |
| hsa-miR-126-4395339 (FAM, NFQ) | 25% | 9.E−02 | 555 |
| hsa-miR-500*-4373225 (FAM, NFQ) | 25% | 9.E−02 | 556 |
| hsa-miR-503-4373228 (FAM, NFQ) | 24% | 1.E−01 | 557 |
| hsa-miR-768-3p-4395188 (FAM, NFQ) | 24% | 1.E−01 | 558 |
| hsa-miR-628-5p-4395544 (FAM, NFQ) | 24% | 1.E−01 | 559 |
| hsa-miR-146b-5p-4373178 (FAM, NFQ) | 23% | 1.E−01 | 560 |
| hsa-miR-455-3p-4395355 (FAM, NFQ) | 23% | 1.E−01 | 561 |
| hsa-miR-574-3p-4395460 (FAM, NFQ) | 23% | 1.E−01 | 562 |
| hsa-miR-99b-4373007 (FAM, NFQ) | 23% | 1.E−01 | 563 |
| hsa-miR-409-3p-4395443 (FAM, NFQ) | 22% | 1.E−01 | 564 |
| hsa-miR-145-4395389 (FAM, NFQ) | 22% | 1.E−01 | 565 |
| hsa-miR-198-4395384 (FAM, NFQ) | 22% | 1.E−01 | 566 |
| hsa-miR-941-4395294 (FAM, NFQ) | 22% | 1.E−01 | 567 |
| hsa-miR-34a*-4395427 (FAM, NFQ) | 21% | 1.E−01 | 568 |
| hsa-miR-379-4373349 (FAM, NFQ) | 21% | 1.E−01 | 569 |
| hsa-miR-195-4373105 (FAM, NFQ) | 21% | 1.E−01 | 570 |
| hsa-miR-125a-3p-4395310 (FAM, NFQ) | 21% | 2.E−01 | 571 |
| hsa-miR-127-3p-4373147 (FAM, NFQ) | 21% | 2.E−01 | 572 |
| hsa-miR-140-3p-4395345 (FAM, NFQ) | 21% | 2.E−01 | 573 |
| hsa-miR-483-5p-4395449 (FAM, NFQ) | 21% | 2.E−01 | 574 |
| hsa-miR-424*-4395420 (FAM, NFQ) | 20% | 2.E−01 | 575 |
| hsa-miR-331-3p-4373046 (FAM, NFQ) | 20% | 2.E−01 | 576 |
| hsa-miR-604-4380973 (FAM, NFQ) | 20% | 2.E−01 | 577 |
| hsa-miR-520g-4373257 (FAM, NFQ) | 20% | 2.E−01 | 578 |
| hsa-miR-877-4395402 (FAM, NFQ) | 20% | 2.E−01 | 579 |
| hsa-miR-921-4395262 (FAM, NFQ) | 20% | 2.E−01 | 580 |
| hsa-miR-199b-5p-4373100 (FAM, NFQ) | 20% | 2.E−01 | 581 |
| hsa-miR-28-5p-4373067 (FAM, NFQ) | 20% | 2.E−01 | 582 |

TABLE 9B shows the 57 miRNAs (SEQ ID NOS:583-639) that were identified from the 700 miRNAs tested which are negatively correlated with EMT/PC1 Signature scores and have a rho score by Pearson analysis of minus 20% or lower, sorted by the EMT p-value (Pearson).

TABLE 9B
MicroRNAS Negatively Correlated to the EMT Signature Score

| | EMT | EMT | |
|---|---|---|---|
| | rho | p-value | |
| Micro RNA Measured | Pearson | Pearson | SEQ ID NO: |
| hsa-miR-518f-4395499 (FAM, NFQ) | −20% | 2.E−01 | 583 |
| hsa-miR-944-4395300 (FAM, NFQ) | −20% | 2.E−01 | 584 |
| hsa-miR-15a-4373123 (FAM, NFQ) | −20% | 2.E−01 | 585 |
| hsa-miR-375-4373027 (FAM, NFQ) | −20% | 2.E−01 | 586 |
| hsa-let-7f-2*-4395529 (FAM, NFQ) | −20% | 2.E−01 | 587 |
| RNU43-4373375 (FAM, NFQ) | −21% | 2.E−01 | 588 |
| hsa-miR-135b*-4395270 (FAM, NFQ) | −21% | 2.E−01 | 589 |
| hsa-miR-20a*-4395548 (FAM, NFQ) | −21% | 2.E−01 | 590 |
| hsa-miR-210-4373089 (FAM, NFQ) | −21% | 1.E−01 | 591 |
| hsa-miR-19b-1*-4395536 (FAM, NFQ) | −21% | 1.E−01 | 592 |
| hsa-miR-629-4395547 (FAM, NFQ) | −21% | 1.E−01 | 593 |
| hsa-miR-101-4395364 (FAM, NFQ) | −21% | 1.E−01 | 594 |
| hsa-miR-801-4395183 (FAM, NFQ) | −21% | 1.E−01 | 595 |
| hsa-miR-449a-4373207 (FAM, NFQ) | −21% | 1.E−01 | 596 |
| hsa-miR-517c-4373264 (FAM, NFQ) | −21% | 1.E−01 | 597 |
| hsa-miR-181a*-4373086 (FAM, NFQ) | −22% | 1.E−01 | 598 |
| hsa-miR-509-5p-4395346 (FAM, NFQ) | −22% | 1.E−01 | 599 |
| hsa-miR-597-4380960 (FAM, NFQ) | −22% | 1.E−01 | 600 |
| hsa-miR-29b-4373288 (FAM, NFQ) | −22% | 1.E−01 | 601 |
| hsa-miR-18b-4395328 (FAM, NFQ) | −22% | 1.E−01 | 602 |
| RNU44-4373384 (FAM, NFQ) | −22% | 1.E−01 | 603 |
| hsa-miR-649-4381005 (FAM, NFQ) | −22% | 1.E−01 | 604 |
| hsa-miR-130b-4373144 (FAM, NFQ) | −22% | 1.E−01 | 605 |
| hsa-miR-7-4378130 (FAM, NFQ) | −24% | 1.E−01 | 606 |
| hsa-miR-30d*-4395416 (FAM, NFQ) | −24% | 1.E−01 | 607 |
| hsa-miR-200c-4395411 (FAM, NFQ) | −24% | 9.E−02 | 608 |
| hsa-miR-519a-4395526 (FAM, NFQ) | −25% | 8.E−02 | 609 |
| hsa-miR-106b*-4395491 (FAM, NFQ) | −25% | 8.E−02 | 610 |
| hsa-miR-922-4395263 (FAM, NFQ) | −25% | 8.E−02 | 611 |
| hsa-miR-645-4381000 (FAM, NFQ) | −27% | 6.E−02 | 612 |
| hsa-miR-15b*-4395284 (FAM, NFQ) | −27% | 6.E−02 | 613 |
| hsa-miR-512-3p-4381034 (FAM, NFQ) | −27% | 6.E−02 | 614 |
| hsa-miR-550-4395521 (FAM, NFQ) | −27% | 6.E−02 | 615 |
| hsa-miR-31-4395390 (FAM, NFQ) | −27% | 6.E−02 | 616 |
| hsa-miR-26a-2*-4395226 (FAM, NFQ) | −27% | 6.E−02 | 617 |
| hsa-miR-148a-4373130 (FAM, NFQ) | −28% | 5.E−02 | 618 |
| hsa-miR-425-4380926 (FAM, NFQ) | −28% | 5.E−02 | 619 |
| hsa-miR-148b-4373129 (FAM, NFQ) | −29% | 4.E−02 | 620 |
| hsa-miR-200b-4395362 (FAM, NFQ) | −29% | 4.E−02 | 621 |
| hsa-miR-449b-4381011 (FAM, NFQ) | −30% | 4.E−02 | 622 |
| hsa-miR-551b*-4395457 (FAM, NFQ) | −30% | 4.E−02 | 623 |
| hsa-miR-141-4373137 (FAM, NFQ) | −30% | 3.E−02 | 624 |
| hsa-miR-147-4373131 (FAM, NFQ) | −31% | 3.E−02 | 625 |
| hsa-miR-141*-4395256 (FAM, NFQ) | −32% | 2.E−02 | 626 |
| hsa-miR-744*-4395436 (FAM, NFQ) | −33% | 2.E−02 | 627 |
| hsa-miR-429-4373203 (FAM, NFQ) | −33% | 2.E−02 | 628 |
| hsa-miR-16-1*-4395531 (FAM, NFQ) | −33% | 2.E−02 | 629 |
| hsa-miR-200a*-4373273 (FAM, NFQ) | −33% | 2.E−02 | 630 |
| hsa-miR-875-5p-4395314 (FAM, NFQ) | −33% | 2.E−02 | 631 |
| hsa-miR-147b-4395373 (FAM, NFQ) | −34% | 2.E−02 | 632 |
| hsa-miR-942-4395298 (FAM, NFQ) | −34% | 2.E−02 | 633 |
| hsa-miR-885-5p-4395407 (FAM, NFQ) | −35% | 1.E−02 | 634 |
| hsa-miR-200b*-4395385 (FAM, NFQ) | −37% | 9.E−03 | 635 |
| hsa-miR-517a-4395513 (FAM, NFQ) | −39% | 6.E−03 | 636 |
| hsa-miR-576-3p-4395462 (FAM, NFQ) | −39% | 6.E−03 | 637 |
| hsa-miR-33a*-4395247 (FAM, NFQ) | −39% | 5.E−03 | 638 |
| hsa-miR-200a-4378069 (FAM, NFQ) | −40% | 4.E−03 | 639 |

Inspection of data in TABLE 9B reveals that of all the micro-RNAs tested, the miR-200 family (including miR-200a, miR-200b, miR-200c, miR-141 and miR-429) was the most highly anti-correlated with corresponding PC1/EMT Signature scores.

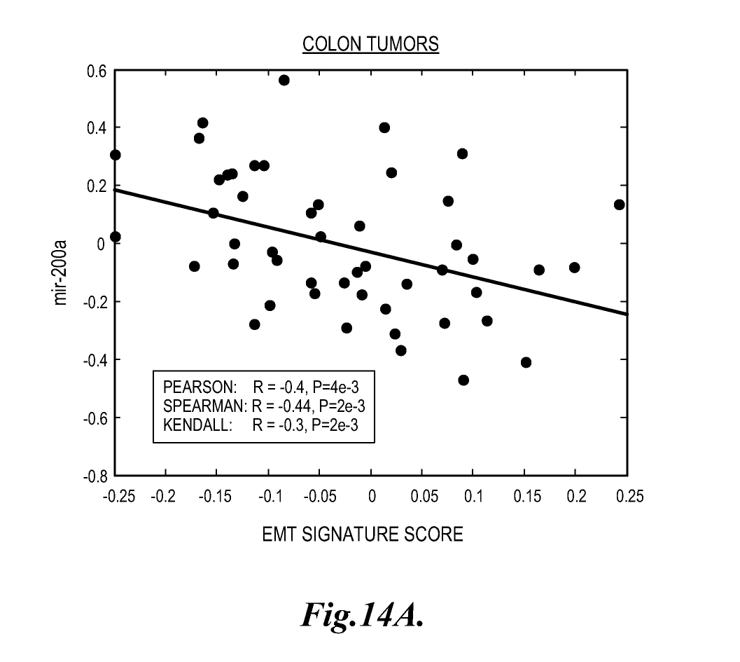

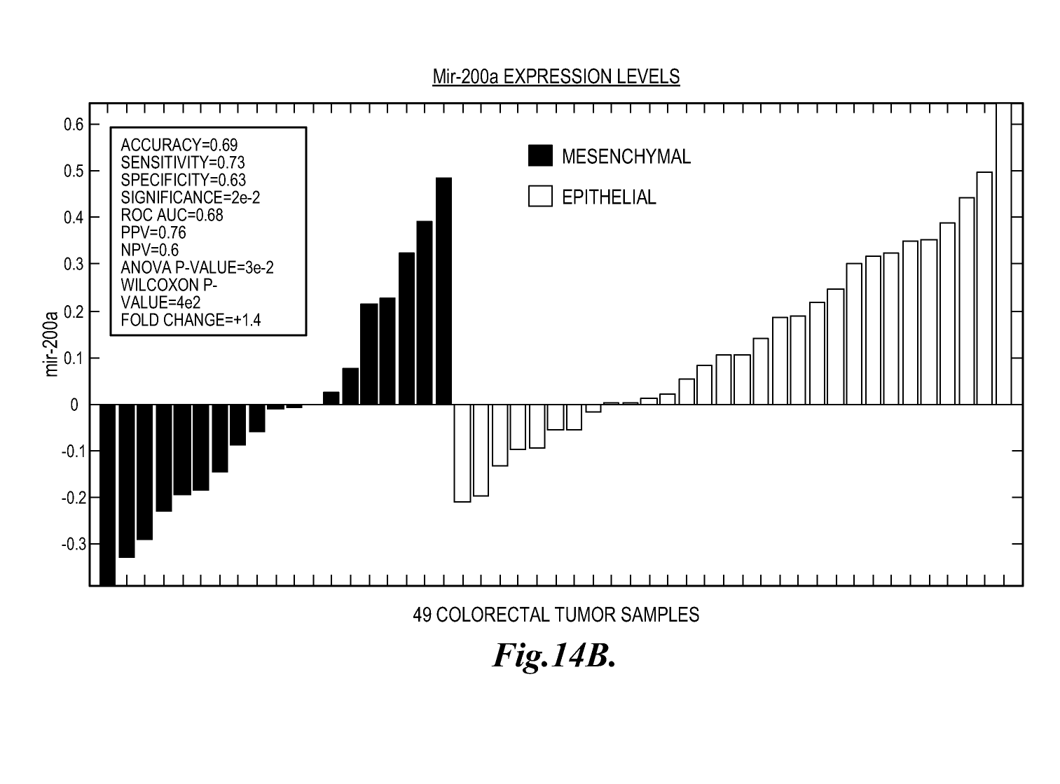

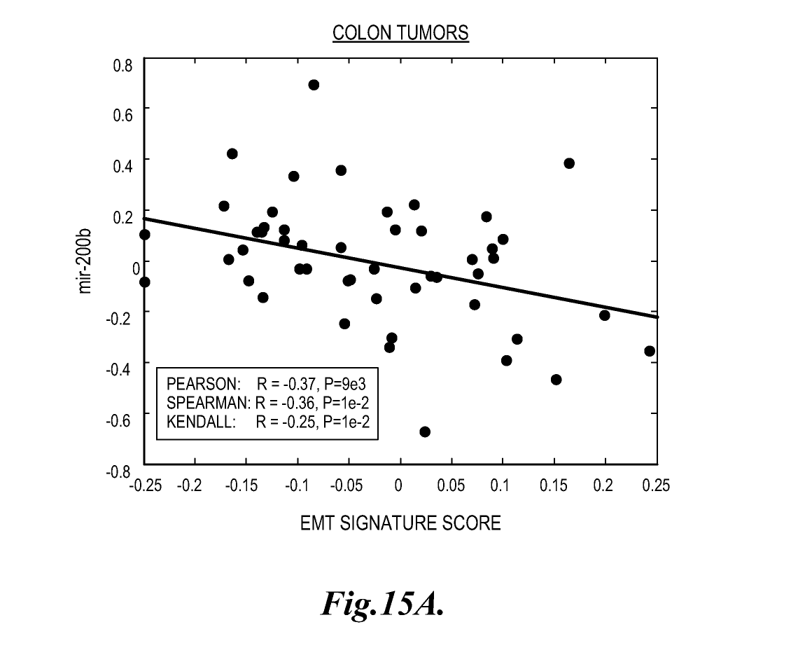

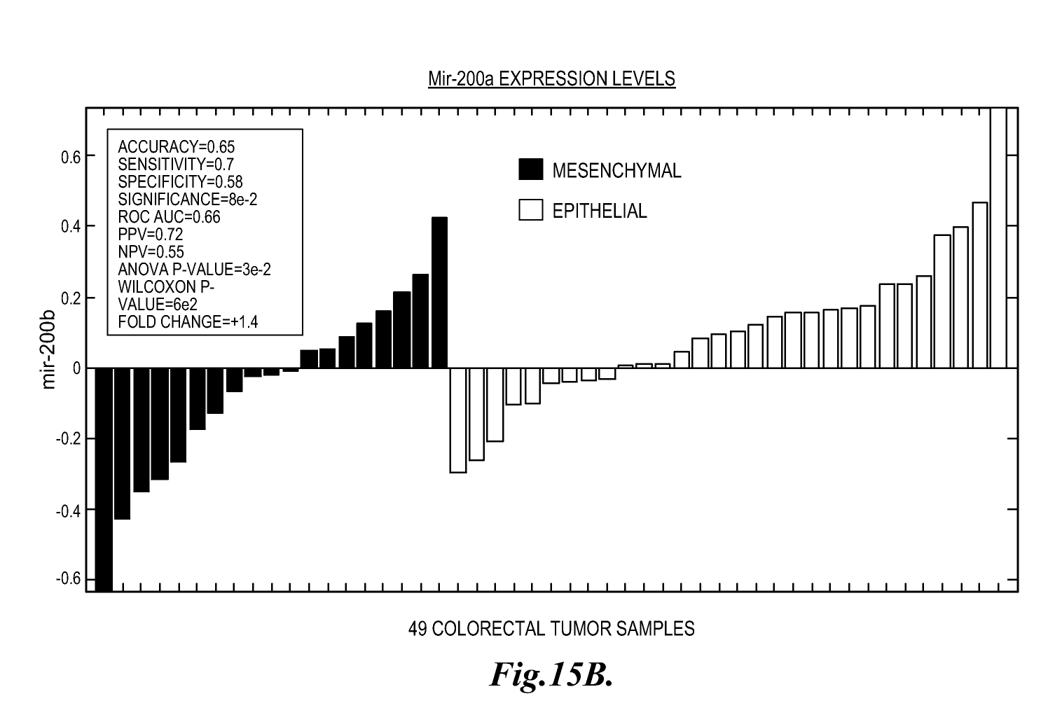

FIG. 14, Panel A shows a plot of the miR-200a measured levels versus corresponding EMT Signature scores across the 49 colorectal cancer samples. FIG. 15, Panel A, shows a plot of the miR-200b measured levels versus corresponding EMT Signature scores across the 49 colorectal cancer samples. Waterfall plots for miR-200a (FIG. 14, Panel B) and miR-200b (FIG. 15, Panel B) show that miR-200 over-expression is correlated with more colon tumors classified as having mesenchymal properties (based on EMT score) than epithelial properties and that miR-200 under expression is correlated with fewer colon tumors classified as having epithelial than mesenchymal properties. The results shown in FIG. 14B have a confusion matrix: TP=22, FP=7, FN=8, TN=12, plotted value=input value−adjustment, adjustment=−0.080685. The results shown in FIG. 15B have a confusion matrix: TP=21, FP=21, FN=9, TN=11, plotted value=input value−adjustment, adjustment=−0.041186.

These finding are significant because the miR-200 family has been closely linked to the EMT program (Gregory et al., 2008, Nat. Cell Biol. 10:593-601; Park et al., 2008, Genes Devel. 22:894-907). It has been previously demonstrated that miR-200 over-expression may result in inhibition of ZEB 1/2, which in turn leads to inhibition of transcriptional repressors of CDH1, thereby permitting the expression of CDH1 and expression of the epithelial phenotype. Thus, a negative correlation of miR-200 levels and the EMT signature genes associated with a mesenchymal tumor phenotype is consistent. The relationship between miR-200 and the PC1 Signature score was strong enough to be detected on a relatively small number of tumors, even when non-mirror image FFPE tissues were used instead of the original frozen specimen, suggesting the EMT program is pervasive throughout the primary tumor. In addition, miR-141, a miR-200 family member, was also identified as negatively correlated with EMT (TABLE 9B) confirming previous observations by Gregory et al. (2008, Nat. Cell Biol. 10:593-601). Finally, there are numerous additional microRNAs that have been identified in TABLE 9B as having significant negative correlations to the EMT Signature score that have not yet been reported to be linked to the EMT program.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for classifying a human subject afflicted with a cancer type which is at risk of undergoing an epithelial cell-like to mesenchymal cell-like transition, as having a good prognosis or a poor prognosis, wherein said good prognosis indicates that said subject is expected to have no distant metastases or no reoccurrence within five years of initial diagnosis of said cancer, and wherein said poor prognosis indicates that said subject is expected to have distant metastases or a reoccurrence of cancer within five years of initial diagnosis of said cancer, the method comprising:
(a) classifying cancer cells obtained from said human subject as having mesenchymal cell-like qualities or epithelial cell-like qualities on the basis of the expression level of at least 5 of the genes for which markers are listed in any of TABLE 2A, TABLE 2B, TABLE 4A, TABLE 4B, and/or of at least one of the microRNAs listed in TABLE 9A and TABLE 9B;
(b) classifying the human subject as having a good prognosis if the cancer cells are classified according to step (a) as having epithelial cell-like properties, or classifying the human subject as having a poor prognosis if the cancer cells are classified according to step (a) as having mesenchymal cell-like properties; and
(c) displaying or outputting to a user, user interface device, computer readable storage medium, or local or remote computer system the classification produced by said classifying step (b).

2. The method of claim 1, wherein said classifying according to step (a) comprises:
(a) calculating a measure of similarity between a first expression profile and a mesenchymal cell-like template, said first expression profile comprising the expression levels of a first plurality of genes in an isolated cell sample derived from said human subject, said mesenchymal cell-like template comprising expression levels of said first plurality of genes that are average expression levels of the respective genes in a plurality of human control cell samples that have mesenchymal cell-like qualities, said first plurality of genes consisting of at least 5 of the genes for which markers are listed in any of TABLE 2A, TABLE 4A and/or at least one of the microRNAs listed in TABLE 9A; and
(b) classifying said cancer cells as having said mesenchymal cell-like properties if said first expression profile has a high similarity to said mesenchymal cell-like template, or classifying said cell sample as having said epithelial cell-like properties if said first expression profile has a low similarity to said mesenchymal cell-like template; wherein said first expression profile has a high similarity to said mesenchymal cell-like template if the similarity to said mesenchymal cell-like template is above a predetermined threshold, or has a low similarity to said mesenchymal cell-like template if the similarity to said mesenchymal cell-like template is below said predetermined threshold.

3. The method of claim 1, wherein said classifying according to step (a) comprises:
(a) calculating a measure of similarity between a first expression profile and an epithelial cell-like template, said first expression profile comprising the expression levels of a first plurality of genes in an isolated cell sample derived from said human subject, said epithelial cell-like template comprising expression levels of said first plurality of genes that are average expression levels of the respective genes in a plurality of human control cell samples that have epithelial cell-like qualities, said first plurality of genes consisting of at least 5 of the genes for which markers are listed in any of TABLE 2B, TABLE 4B, and/or at least one of the microRNAs listed in TABLE 9B; and
(b) classifying said cancer cells as having said epithelial cell-like properties if said first expression profile has a high similarity to said epithelial cell-like template, or classifying said cell sample as having said mesenchymal cell-like properties if said first expression profile has a low similarity to said epithelial cell-like template; wherein said first expression profile has a high similarity to said epithelial cell-like template if the similarity to said epithelial cell-like template is above a predetermined threshold, or has a low similarity to said epithelial cell-like template if the similarity to said epithelial cell-like template is below said predetermined threshold.

4. The method of claim 1, wherein said classifying according to step (a) comprises calculating an EMT Signature Score for the cancer cells isolated from the human subject by a method comprising:
(a) calculating a differential expression value of a first expression level of each of a first plurality of genes and each of a second plurality of genes in the isolated cancer cell sample derived from the human subject relative to a second expression level of each of said first plurality of genes and each of said second plurality of genes in a human control cell sample, said first plurality of genes consisting of at least 5 of the genes for which markers are listed in any of TABLES 2A, 4A, and/or at least one of the microRNAs listed in TABLE 9A (mesenchymal arm) and said second plurality of genes consisting of at least 5 of the genes for which markers are listed in any of TABLES 2B, 4B, and/or at least one of the microRNAs listed in TABLE 9B (epithelial arm);
(b) calculating the mean differential expression values of the expression levels of said first plurality of genes and said second plurality of genes;
(c) subtracting said mean differential expression value of said second plurality of genes from said mean differential expression value of said first plurality of genes to obtain said EMT Signature Score; and
(d) classifying said cancer cell sample as having mesenchymal cell-like properties if said obtained EMT Signature Score is at or above a first predetermined threshold and is statistically significant, or classifying said cancer cell sample as having epithelial cell-like properties if said obtained EMT Signature Score is at or below a second predetermined threshold and is statistically significant.

5. The method of claim 4, wherein said first plurality consists of at least 6, 7, 8, 9, 10, 11, 12, 13, 14, 15, 16, 17, 18, 19, or 20 of the genes for which markers are listed in TABLE 2A.

6. The method of claim 4, wherein said second plurality consists of at least 6, 7, 8, 9, 10, 11, 12, 13, 14, 15, 16, 17, 18, 19, or 20 of the genes for which markers are listed in TABLE 2B.

7. The method of claim 4, wherein said first plurality consists of all the genes for which markers are listed in TABLE 2A.

8. The method of claim 4, wherein said second plurality consists of all the genes for which markers are listed in TABLE 2B.

9. The method of claim 1, wherein said classifying according to step (a) comprises calculating a PC1 Signature Score for the cancer cells isolated from the human subject by a method comprising:
(a) calculating a differential expression value of a first expression level of each of a first plurality of genes and each of a second plurality of genes in the isolated cancer cell sample derived from the human subject relative to a second expression level of each of said first plurality of genes and each of said second plurality of genes in a human control cell sample, said first plurality of genes consisting of at least 5 of the genes for which markers are listed in TABLE 4A (mesenchymal arm) and said second plurality of genes consisting of at least 5 of the genes for which markers are listed in TABLE 4B (epithelial arm);
(b) calculating the mean differential expression values of the expression levels of said first plurality of genes and said second plurality of genes;
(c) subtracting said mean differential expression value of said second plurality of genes from said mean differential expression value of said first plurality of genes to obtain said PC1 Signature Score; and
(d) classifying said cancer cell sample as having mesenchymal cell-like properties if said obtained PC1 Signature Score is at or above a first predetermined threshold and is statistically significant; or classifying said cancer cell sample as having epithelial cell-like properties if said obtained PC1 Signature Score is at or below a second predetermined threshold and is statistically significant.
